(19) United States
(12) Patent Application Publication
(10) Pub. No.: US 2023/0120895 A1
(43) Pub. Date: Apr. 20, 2023

(54) PESTICIDALLY ACTIVE HETEROCYCLIC DERIVATIVES WITH SULFUR CONTAINING SUBSTITUENTS

(71) Applicant: SYNGENTA CROP PROTECTION AG, Basel (CH)

(72) Inventors: Vikas SIKERVAR, Goa (IN); Indira SEN, Goa (IN); Michel MUEHLEBACH, Stein (CH); Sebastian RENDLER, Basel (CH); André STOLLER, Stein (CH); Daniel EMERY, Stein (CH); Anke BUCHHOLZ, Stein (CH); Benedikt KURTZ, Stein (CH)

(73) Assignee: SYNGENTA CROP PROTECTION AG, Basel (CH)

(21) Appl. No.: 17/790,069

(22) PCT Filed: Dec. 22, 2020
(86) PCT No.: PCT/EP2020/087613

§ 371 Date: Jun. 29, 2022

(30) Foreign Application Priority Data
Dec. 31, 2019 (IN) 201911054729

(51) Int. Cl. A01N 43/40; C07D 471/04; A01P 7/02; A01P 7/04; A01P 9/00; A01P 5/00

(52) U.S. Cl. CPC A01N 43/40; C07D 471/04; A01P 7/02; A01P 7/04; A01P 9/00; A01P 5/00

(57) ABSTRACT

Compounds of the formula (I) wherein the substituents are as defined in claim 1. Furthermore, the present invention relates to agrochemical compositions which comprise compounds of formula (I), to preparation of these compositions, and to the use of the compounds or compositions in agriculture or horticulture for combating, preventing or controlling animal pests, including arthropods and in particular insects, nematodes, molluscs or representatives of the order Acarina.

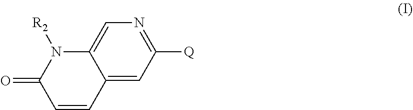

The present invention relates to pesticidally active, in particular insecticidally active heterocyclic derivatives containing sulfur substituents, to processes for their preparation, to compositions comprising those compounds, and to their use for controlling animal pests, including arthropods and in particular insects or representatives of the order Acarina.

Heterocyclic compounds with pesticidal action are known and described, for example, in WO2013191112.

It has now surprisingly been found that certain novel pesticidally active derivatives with sulfur containing substitutents have favourable properties as pesticides.

The present invention therefore provides compounds of formula I,

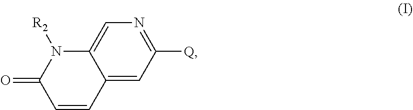

wherein

R₂ is C₁-C₆haloalkyl;

Q is a radical selected from the group consisting of formula Qa and Qb

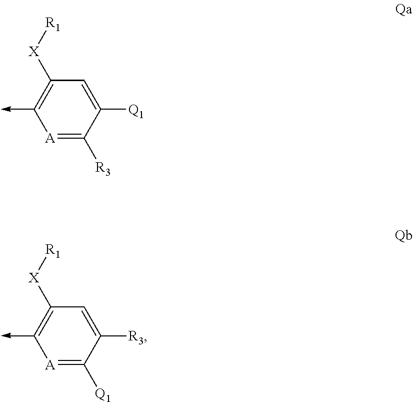

wherein the arrow denotes the point of attachment to the carbon atom of the bicyclic ring; and wherein A represents CH or N;

X is S, SO, or SO₂;

R₁ is C₁-C₄alkyl or C₃-C₆cycloalkyl-C₁-C₄alkyl;

Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system can contain 1, 2 or 3 ring heteroatoms selected from the group consisting of nitrogen, oxygen and sulphur, where said ring system may not contain more than one ring oxygen atom and not more than one ring sulfur atom; or

Q₁ is a five-membered aromatic ring system linked via a ring nitrogen atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system contains 1, 2 or 3 ring heteroatoms selected from the group consisting of nitrogen, oxygen and sulphur, where said ring system contains at least one ring nitrogen atom and may not contain more than one ring oxygen atom and not more than one ring sulfur atom;

R₃ is hydrogen, halogen or C₁-C₄alkyl;

Each R₄ is independently hydrogen, C₁-C₄alkyl or C₃-C₆cycloalkyl; and

R₅ is C₁-C₆alkyl, C₁-C₆haloalkyl or C₃-C₆cycloalkyl.

The present invention also provides agrochemically acceptable salts, stereoisomers, enantiomers, tautomers and N-oxides of the compounds of formula I.

Compounds of formula I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula I according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula I according to the invention also include hydrates which may be formed during the salt formation.

Where substituents are indicated as being itself further substituted, this means that they carry one or more identical or different substituents, e.g. one to four substituents. Normally not more than three such optional substituents are present at the same time. Preferably not more than two such substituents are present at the same time (i.e. the group is substituted by one or two of the substituents indicated). Where the additional substituent group is a larger group, such as cycloalkyl or phenyl, it is most preferred that only one such optional substituent is present. Where a group is indicated as being substituted, e.g. alkyl, this includes those groups that are part of other groups, e.g. the alkyl in alkylthio.

The term “C₁-Cₙalkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2,2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₁-Cₙhaloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂-fluoroalkyl” would refer to a C₁-C₂-alkyl radical which carries 1,2, 3,4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or penta-fluoroethyl.

The term “C₁-Cₙalkoxy” as used herein refers to a straight-chain or branched saturated alkyl radical having 1 to n carbon atoms (as mentioned above) which is attached via an oxygen atom, i.e., for example, any one of methoxy, ethoxy, n-propoxy, 1-methylethoxy, n-butoxy, 1-methylpropoxy, 2-methylpropoxy or 1,1-dimethylethoxy.

The term “C₁-Cₙhaloalkoxy” as used herein refers to a C₁-Cₙalkoxy radical as mentioned above which is partially or fully substituted by fluorine, chlorine, bromine and/or iodine, i.e., for example, any one of chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2, 3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, or 4-bromobutoxy.

The term “C₁-Cₙ-alkylsulfanyl” as used herein refers to a straight chain or branched saturated alkyl radical having 1 to n carbon atoms (as mentioned above) which is attached via a sulfur atom, i.e., for example, any one of methylthio, ethylthio, n-propylthio, 1-methylethylthio, butylthio, 1-methylpropylthio, 2-methylpropylthio or 1,1-dimethylethylthio.

The term “C₁-Cₙalkylsulfinyl” as used herein refers to a straight chain or branched saturated alkyl radical having 1 to n carbon atoms (as mentioned above) which is attached via the sulfur atom of the sulfinyl group, i.e., for example, any one of methylsulfinyl, ethylsulfinyl, n-propylsulfinyl, 1-methylethyl-sulfinyl, n-butylsulfinyl, 1-methylpropylsulfinyl, 2-methylpropylsulfinyl, 1,1-dimethyl-ethylsulfinyl, n-pentylsulfinyl, 1-methylbutylsulfinyl, 2-methylbutylsulfinyl, 3-methyl-butylsulfinyl, 1,1-dimethylpropylsulfinyl, 1,2-dimethylpropylsulfinyl, 2,2-dimethylpropylsulfinyl or 1-ethylpropylsulfinyl.

The term “C₁-Cₙalkylsulfonyl” as used herein refers to a straight chain or branched saturated alkyl radical having 1 to n carbon atoms (as mentioned above) which is attached via the sulfur atom of the sulfonyl group, i.e., for example, any one of methylsulfonyl, ethylsulfonyl, n-propylsulfonyl, isopropylsulfonyl, n-butylsulfonyl, 1-methylpropylsulfonyl, 2-methylpropylsulfonyl ort-butylsulphonyl.

The term “C₁-Cₙcyanoalkyl” as used herein refers to a straight chain or branched saturated alkyl radicals having 1 to n carbon atoms (as mentioned above) which is substituted by a cyano group, for example cyanomethylene, cyanoethylene, 1,1-dimethylcyanomethyl, cyanomethyl, cyanoethyl, and 1-dimethylcyanomethyl.

The term “C₁-Cₙcyanoalkoxy” refers to the groups above but which is attached via an oxygen atom.

The suffix “—C₁-Cₙalkyl” after terms such as “C₃-Cₙcycloalkyl”, wherein n is an integer from 1-6, as used herein refers to a straight chain or branched saturated alkyl radicals which is substituted by C₃-Cₙcycloalkyl. An example of C₃-Cₙcycloalkyl-C₁-Cₙalkyl is for example, cyclopropylmethyl.

The term “C₃-C₆cycloalkyl” as used herein refers to 3-6 membered cycloylkyl groups such as cyclopropane, cyclobutane, cyclopropane, cyclopentane and cyclohexane.

Halogen is generally fluorine, chlorine, bromine or iodine. This also applies, correspondingly, to halogen in combination with other meanings, such as haloalkyl.

In the context of this invention “mono- or polysubstituted” in the definition of the substituents, means typically, depending on the chemical structure of the substituents, monosubstituted to five-times substituted, more preferably mono-, double- or triple-substituted.

In the context of the this invention, the phrases “Q₁ is a five- to six-membered aromatic ring system, linked via a ring carbon atom to the ring which contains the substituent A . . . ” and “Q₁ is a five-membered aromatic ring system linked via a ring nitrogen atom to the ring which contains the substituent A . . . ”, as the case may be, refer to the manner of attachment of particular embodiments of the substituent Q₁ to the radical Q as represented by either formula Qa or formula Qb, as the case may be. This also applies, correspondingly, to the pyridyl and phenyl embodiments when A is N or C, respectively (i.e., to the pyridyl ring substituted by X—R₁ or to the phenyl ring substituted by X—R₁).

In the context of this invention, examples of “Q₁ is a five- to six-membered aromatic ring system, linked via a ring carbon atom to the ring which contains the substituent A, . . . , and said ring system can contain 1, 2 or 3 heteroatoms” are, but not limited to, phenyl, pyrazolyl, triazolyl, pyridinyl and pyrimidinyl; preferably phenyl, 2-pyridyl, 3-pyridyl, 4-pyridyl, pyrimidin-2-yl, pyrimidin-4-yl, and pyrimidin-5-yl. This also applies, correspondingly, to the pyridyl and phenyl embodiments when A is N or C, respectively.

In the context of this invention, examples of “Q₁ is a five-membered aromatic ring system linked via a ring nitrogen atom to the ring which contains the substituent A, . . . , and said ring system contains 1, 2 or 3 heteroatoms” are, but not limited to, pyrazolyl, pyrrolyl, imidazolyl and triazolyl; preferably pyrrol-1-yl, pyrazol-1-yl, triazol-2-yl, 1,2,4-triazol-1-yl, triazol-1-yl, and imidazol-1-yl. This also applies, correspondingly, to the pyridyl and phenyl embodiments when A is N or C, respectively.

Certain embodiments according to the invention are provided as set out below.

Embodiment 1 provides compounds of formula I, or an agrochemically acceptable salt, stereoisomer, enantiomer, tautomer or N-oxide thereof, as defined above.

Embodiment 2 provides compounds, or an agrochemically acceptable salt, stereoisomer, enantiomer, tautomer or N-oxide thereof, according to embodiment 1 wherein Q is Qa and having preferred values of R₂, A, X, R₁, Q₁, R₄, R₅ and R₃ as set out below.

Embodiment 3 provides compounds, or an agrochemically acceptable salt, stereoisomer, enantiomer, tautomer or N-oxide thereof, according to embodiment 1 wherein Q is Qb and having preferred values of R₂, A, X, R₁, Q₁, R₄, R₅ and R₃ as set out below.

With respect to embodiments 1-3, preferred values of R₂, A, X, R₁, Q₁, R₄, R₅ and R₃ are, in any combination thereof, as set out below:

Preferably R₂ is C₁-C₆haloalkyl.

More preferably R₂ is C₁-C₆fluoroalkyl.

Even more preferably R₂ is —CH₂CF₂CF₃, —CH₂CF₂CHF₂, —CH₂CF₃, —CH₂CHF₂ or —CH₂CF₂CHFCF₃.

Most preferably R₂ is —CH₂CF₃, —CH₂CF₂CHF₂ or —CH₂CF₂CF₃.

Preferably A is N.

Preferably X is S or SO₂.

Most preferably X is SO₂.

Preferably R₁ is C₁-C₄alkyl or cyclopropyl-C₁-C₄alkyl.

More preferably R₁ is ethyl or cyclopropylmethyl.

Most preferably R₁ is ethyl.

Preferably Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms.

More preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl.

More preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Most preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Preferably each R₄ is independently hydrogen or C₁-C₄alkyl.

Most preferably each R₄ is independently hydrogen or methyl.

Preferably R₅ is C₁-C₆alkyl or C₃-C₆cycloalkyl.

More preferably R₅ is methyl, ethyl or cyclopropyl.

More preferably R₅ is methyl or cyclopropyl.

Most preferably R₅ is methyl.

Preferably R₃ is hydrogen or C₁-C₄alkyl.

More preferably R₃ is hydrogen or methyl.

Most preferably R₃ is hydrogen.

One group of compounds according to the invention are those of formula 1-1

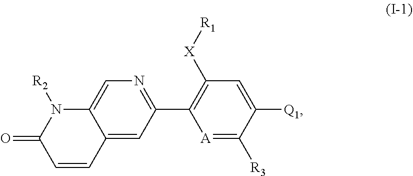

wherein A, X, R₁, and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;

R₃ is preferably hydrogen or C₁-C₄alkyl;

Preferably each R₄ is independently hydrogen or C₁-C₄alkyl; and

R₅ is preferably C₁-C₆alkyl or C₃-C₆cycloalkyl.

Preferred definitions of A, X, R₁, and R₂ in the compounds of formula 1-1 are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-1a) which are compounds of formula (I-1) wherein A is N.

Another group of compounds according to this embodiment are compounds of formula (I-1 b) which are compounds of formula (I-1) wherein A is CH.

One group of compounds according to this embodiment are compounds of formula (I-1c) which are compounds of formula (I-1) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₂CHF₂ or —CH₂CF₂CF₃.

Another group of compounds according to this embodiment are compounds of formula (I-1d) which are compounds of formula (I-1) wherein X is S or SO₂., preferably X is SO₂.

Another group of compounds according to this embodiment are compounds of formula (I-1e) which are compounds of formula (I-1) wherein R₁ is C₁-C₄alkyl or cyclopropyl-C₁-C₄alkyl; preferably R₁ is ethyl or cyclopropylmethyl; more preferably R₁ is ethyl.

Another group of compounds according to the invention are those of formula 1-2

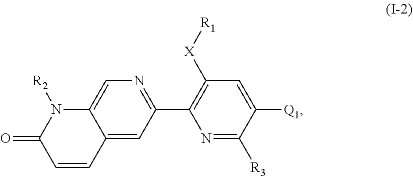

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;

R₃ is preferably hydrogen or C₁-C₄alkyl;

Preferably each R₄ is independently hydrogen or C₁-C₄alkyl; and

R₅ is preferably C₁-C₆alkyl or C₃-C₆cycloalkyl.

Preferred definitions of X, R₁ and R₂ in the compounds of formula 1-2 are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-2a) which are compounds of formula (I-2) wherein X is S or SO₂, preferably X is SO₂.

Another group of compounds according to this embodiment are compounds of formula (I-2b) which are compounds of formula (I-2) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-2c) which are compounds of formula (I-2) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₂CHF₂ or —CH₂CF₂CF₃.

Another group of compounds according to the invention are those of formula 1-3

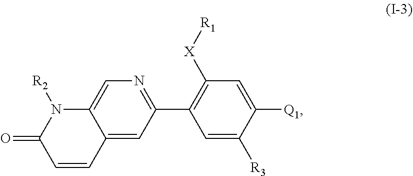

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;

R₃ is preferably hydrogen or C₁-C₄alkyl;

Preferably each R₄ is independently hydrogen or C₁-C₄alkyl; and

R₅ is preferably C₁-C₆alkyl or C₃-C₆cycloalkyl.

Preferred definitions of X, R₁ and R₂ in the compounds of formula 1-3 are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-3a) which are compounds of formula (I-3) wherein X is S or SO₂, preferably X is SO₂.

Another group of compounds according to this embodiment are compounds of formula (I-3b) which are compounds of formula (I-3) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-3c) which are compounds of formula (I-3) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₂CHF₂ or —CH₂CF₂CF₃.

Another group of compounds according to the invention are those of formula 1-4

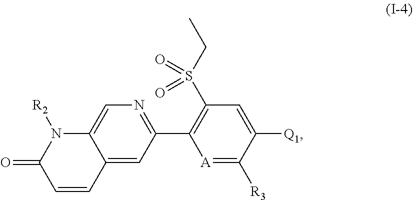

wherein

A is CH or N, preferably N;

R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;

R₃ is hydrogen or C₁-C₄alkyl, preferably hydrogen or methyl;

Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;

Each R₄ is independently hydrogen or C₁-C₄alkyl, preferably hydrogen or methyl; and R₅ is C₁-C₆alkyl or C₃-C₆cycloalkyl, preferably methyl, ethyl or cyclopropyl, more preferably methyl or cyclopropyl; or

an agrochemically acceptable salt, stereoisomer, enantiomer, tautomer or N-oxide thereof.

One further preferred group of compounds according to this embodiment are compounds of formula (I-4a) which are compounds of formula (I-4) wherein A is N.

Another preferred group of compounds according to this embodiment are compounds of formula (I-4b) which are compounds of formula (I-4) wherein A is CH.

One further preferred group of compounds according to this embodiment are compounds of formula (I-4c) which are compounds of formula (I-4) wherein R₃ is hydrogen.

Another preferred group of compounds according to this embodiment are compounds of formula (I-4d) which are compounds of formula (I-4) wherein R₃ is C₁-C₄alkyl, preferably methyl.

One further preferred group of compounds according to this embodiment are compounds of formula (I-4e) which are compounds of formula (I-4) wherein A is N and R₃ is hydrogen.

One further preferred group of compounds according to this embodiment are compounds of formula (I-40 which are compounds of formula (I-4) wherein Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is either methyl, ethyl or cyclopropyl, or Q₁ is (oxazolidin-2-one)-3-yl or 2-pyridyloxy; preferably Q₁ is is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, or 2-pyridyloxy; more preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, or 2-pyridyloxy.

One further preferred group of compounds according to this embodiment are compounds of formula (I-4 g) which are compounds of formula (I-4) wherein Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; preferably Q₁ is C-linked pyrimidinyl; more preferably Q₁ is pyrimidin-2-yl.

One further preferred group of compounds according to this embodiment are compounds of formula (I-4h) which are compounds of formula (I-4) wherein Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl; more preferably Q₁ is pyrazol-1-yl, 3-chloro-pyrazol-1-yl, 3-cyano-pyrazol-1-yl, 3-trifluoromethyl-pyrazol-1-yl or 1,2,4-triazol-1-yl.

One further preferred group of compounds according to this embodiment are compounds of formula (I-4i) which are compounds of formula (I-4) wherein

A is N;

R₂ is C₁-C₆fluoroalkyl, preferably —CH₂CF₂CHF₂ or —CH₂CF₂CF₃;

R₃ is hydrogen or C₁-C₄alkyl, preferably hydrogen or methyl; and

Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl.

Another preferred group of compounds according to this embodiment are compounds of formula (I-4j) which are compounds of formula (I-4) wherein

A is N;

R₂ is —CH₂CF₂CHF₂ or —CH₂CF₂CF₃;

R₃ is hydrogen; and

Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to the invention are those of formula 1-5

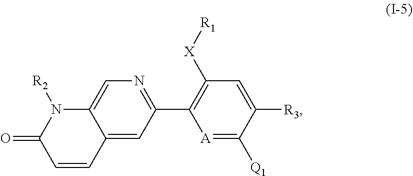

wherein A, X, R₁, and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;

R₃ is preferably hydrogen or C₁-C₄alkyl;

Preferably each R₄ is independently hydrogen or C₁-C₄alkyl; and

R₅ is preferably C₁-C₆alkyl or C₃-C₆cycloalkyl.

Preferred definitions of A, X, R₁, and R₂ in the compounds of formula 1-5 are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-5a) which are compounds of formula (I-5) wherein A is N.

Another group of compounds according to this embodiment are compounds of formula (I-5b) which are compounds of formula (I-5) wherein A is CH.

One group of compounds according to this embodiment are compounds of formula (I-5c) which are compounds of formula (I-5) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₂CHF₂ or —CH₂CF₂CF₃.

Another group of compounds according to this embodiment are compounds of formula (I-5d) which are compounds of formula (I-5) wherein X is S or SO₂, preferably X is SO₂.

Another group of compounds according to this embodiment are compounds of formula (I-5e) which are compounds of formula (I-5) wherein R₁ is C₁-C₄alkyl or cyclopropyl-C₁-C₄alkyl; preferably R₁ is ethyl or cyclopropylmethyl; more preferably R₁ is ethyl.

Another group of compounds according to the invention are those of formula 1-6

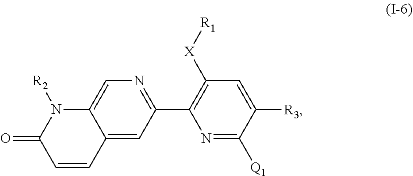

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;

R₃ is preferably hydrogen or C₁-C₄alkyl;

Preferably each R₄ is independently hydrogen or C₁-C₄alkyl; and

R₅ is preferably C₁-C₆alkyl or C₃-C₆cycloalkyl.

Preferred definitions of X, R₁ and R₂ in the compounds of formula 1-6 are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-6a) which are compounds of formula (I-6) wherein X is S or SO₂, preferably X is SO₂.

Another group of compounds according to this embodiment are compounds of formula (I-6b) which are compounds of formula (I-6) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-6c) which are compounds of formula (I-6) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₂CHF₂ or —CH₂CF₂CF₃.

Another group of compounds according to the invention are those of formula 1-7

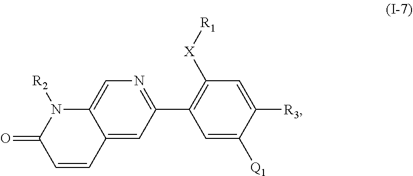

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;

R₃ is preferably hydrogen or C₁-C₄alkyl;

Preferably each R₄ is independently hydrogen or C₁-C₄alkyl; and

R₅ is preferably C₁-C₆alkyl or C₃-C₆cycloalkyl.

Preferred definitions of X, R₁ and R₂ in the compounds of formula 1-7 are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; and R₃ is hydrogen or methyl, preferably hydrogen.

One group of compounds according to this embodiment are compounds of formula (I-7a) which are compounds of formula (I-7) wherein X is S or SO₂, preferably X is SO₂.

Another group of compounds according to this embodiment are compounds of formula (I-7b) which are compounds of formula (I-7) wherein R₁ is C₁-C₄alkyl or cyclopropyl-C₁-C₄alkyl, preferably R₁ is ethyl or cyclopropylmethyl; more preferably R₁ is ethyl.

Another group of compounds according to this embodiment are compounds of formula (I-7c) which are compounds of formula (I-7) wherein R₂ is C₁-C₆fluoroalkyl; preferably R₂ is —CH₂CF₂CF₃, —CH₂CF₂CHF₂, —CH₂CF₃, —CH₂CHF₂ or —CH₂CF₂CHFCF₃; more preferably R₂ is —CH₂CF₂CHF₂ or —CH₂CF₂CF₃.

Another group of compounds according to the invention are those of formula 1-8

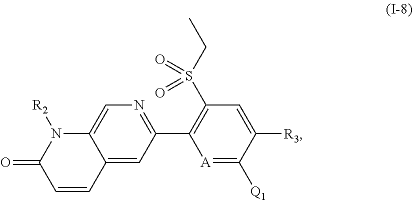

wherein

A is CH or N, preferably N;

R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;

R₃ is hydrogen or C₁-C₄alkyl, preferably hydrogen or methyl;

Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;

Each R₄ is independently hydrogen or C₁-C₄alkyl, preferably hydrogen or methyl; and

R₅ is C₁-C₆alkyl or C₃-C₆cycloalkyl; preferably methyl, ethyl or cyclopropyl, more preferably methyl or cyclopropyl; or

an agrochemically acceptable salt, stereoisomer, enantiomer, tautomer or N-oxide thereof.

One further preferred group of compounds according to this embodiment are compounds of formula (I-8a) which are compounds of formula (I-8) wherein A is N.

Another preferred group of compounds according to this embodiment are compounds of formula (I-8b) which are compounds of formula (I-8) wherein A is CH.

One further preferred group of compounds according to this embodiment are compounds of formula (I-8c) which are compounds of formula (I-8) wherein R₃ is hydrogen.

Another preferred group of compounds according to this embodiment are compounds of formula (I-8d) which are compounds of formula (I-8) wherein R₃ is C₁-C₄alkyl, preferably methyl.

One further preferred group of compounds according to this embodiment are compounds of formula (I-8e) which are compounds of formula (I-8) wherein A is N and R₃ is hydrogen.

One further preferred group of compounds according to this embodiment are compounds of formula (I-80 which are compounds of formula (I-8) wherein Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl, or Q₁ is (oxazolidin-2-one)-3-yl or 2-pyridyloxy; preferably Q₁ is is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, or 2-pyridyloxy; more preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, or 2-pyridyloxy.

One further preferred group of compounds according to this embodiment are compounds of formula (I-8 g) which are compounds of formula (I-8) wherein Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; preferably Q₁ is C-linked pyrimidinyl; more preferably Q₁ is pyrimidin-2-yl.

One further preferred group of compounds according to this embodiment are compounds of formula (I-8h) which are compounds of formula (I-8) wherein Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl; more preferably Q₁ is pyrazol-1-yl, 3-chloro-pyrazol-1-yl, 3-cyano-pyrazol-1-yl, 3-trifluoromethyl-pyrazol-1-yl or 1,2,4-triazol-1-yl.

One further preferred group of compounds according to this embodiment are compounds of formula (I-8i) which are compounds of formula (I-8) wherein

A is N;

R₂ is C₁-C₆fluoroalkyl, preferably —CH₂CF₂CHF₂ or —CH₂CF₂CF₃;

R₃ is hydrogen or C₁-C₄alkyl, preferably hydrogen or methyl; and

Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl.

Another preferred group of compounds according to this embodiment are compounds of formula (I-8j) which are compounds of formula (I-8) wherein

A is N;

R₂ is —CH₂CF₂CHF₂ or —CH₂CF₂CF₃;

R₃ is hydrogen; and

Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

An outstanding group of compounds according to the invention are those of formula 1-9

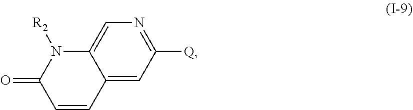

wherein

R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;

Q is a radical selected from the group consisting of formula Qa1 and Qb1

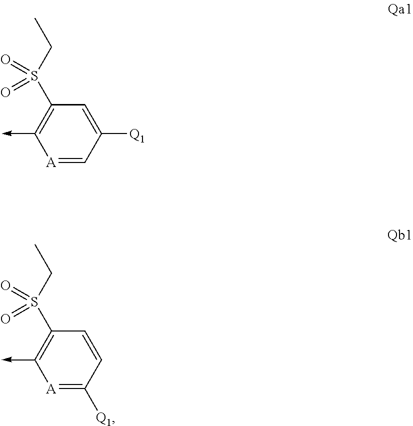

wherein the arrow denotes the point of attachment to the carbon atom of the bicyclic ring;

and wherein

A is CH or N, preferably N; and

Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl; preferably R₅ is methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl;

more preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One group of compounds according to this embodiment are compounds of formula (I-9a) which are compounds of formula (I-9) and any of the preferred embodiments of formula (I-9) wherein A is N.

Another group of compounds according to this embodiment are compounds of formula (I-9b) which are compounds of formula (I-9) and any of the preferred embodiments of formula (I-9) wherein A is CH.

One group of compounds according to this embodiment are compounds of formula (I-9c) which are compounds of formula (I-9) and any of the preferred embodiments of formula (I—9) wherein R₂ is —CH₂CF₂CHF₂ or —CH₂CF₂CF₃.

Another group of compounds according to this embodiment are compounds of formula (I-9d) which are compounds of formula (I-9) and any of the preferred embodiments of formula (I-9) wherein Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is methyl, ethyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl, or Q₁ is N-linked triazolyl or C-linked pyrimidinyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Another group of compounds according to this embodiment are compounds of formula (I-9e) which are compounds of formula (I-9) and any of the preferred embodiments of formula (I-9) wherein Q₁ is hydrogen.

Another group of compounds according to this embodiment are compounds of formula (I-90 which are compounds of formula (I-9) and any of the preferred embodiments of formula (I-9) wherein Q₁ is chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), or —N(CH₃)CONH(CH₃).

Another group of compounds according to this embodiment are compounds of formula (I-9 g) which are compounds of formula (I-9) and any of the preferred embodiments of formula (I-9) wherein Q₁ is (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One further outstanding group of compounds according to this embodiment are compounds of formula (I-9h) which are compounds of formula (I-9) wherein:

R₂ is —CH₂CF₂CHF₂ or —CH₂CF₂CF₃;

Q is a radical selected from the group consisting of formula Qa1 and Qb1, wherein

A is N; and

Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

One further outstanding group of compounds according to this embodiment are compounds of formula (I-9h-1) which are compounds of formula (I-9) wherein:

R₂ is —CH₂CF₂CF₃ or —CH₂CF₃;

Q is a radical selected from the group consisting of formula Qa1 and Qb1, wherein

A is N; and

Q₁ is hydrogen, chlorine, cyclopropyl, 1-cyanocyclopropyl, 1-cyano-1-methyl-ethyl, —NH(CH₃), —N(CH₃)COCH3, 2-pyridyloxy, 3-chloro-pyrazol-1-yl, 1,2,4-triazol-1-yl, triazol-1-yl or triazol-2-yl.

One further outstanding group of compounds according to this embodiment are compounds of formula (I-9h-2) which are compounds of formula (I-9) wherein:

R₂ is —CH₂CF₂CF₃ or —CH₂CF₃;

Q is a radical selected from the group consisting of formula Qa1 and Qb1, wherein

A is N; and

Q₁ is hydrogen, chlorine, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl, or 1,2,4-triazol-1-yl.

One further outstanding group of compounds according to this embodiment are compounds of formula (I-9i) which are compounds of formula (I-9) wherein

R₂ is —CH₂CF₂CHF₂ or —CH₂CF₂CF₃;

Q is a radical selected from the group consisting of formula Qa1 and Qb1, wherein

A is N; and

Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl when Q is Qa1; or

Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl when Q is Qb1.

One further outstanding group of compounds according to this embodiment are compounds of formula (I-9j) which are compounds of formula (I-9) wherein

R₂ is —CH₂CF₂CF₃ or —CH₂CF₃;

Q is the radical Qa1, wherein

A is N; and

Q₁ is hydrogen, chlorine, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl, 1,2,4-triazol-1-yl, triazol-1-yl or triazol-2-yl.

One further outstanding group of compounds according to this embodiment are compounds of formula (I-9j-1) which are compounds of formula (I-9j) wherein Q₁ is hydrogen, 1-cyanocyclopropyl, 1-cyano-1-methyl-ethyl, —NH(CH₃), —N(CH₃)COCH₃, 2-pyridyloxy, or 3-chloro-pyrazol-1-yl.

One further outstanding group of compounds according to this embodiment are compounds of formula (I-9k) which are compounds of formula (I-9) wherein

R₂ is —CH₂CF₂CF₃ or —CH₂CF₃;

Q is the radical Qb1, wherein

A is N; and

Q₁ is hydrogen, chlorine, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl, 1,2,4-triazol-1-yl, triazol-1-yl or triazol-2-yl.

One further outstanding group of compounds according to this embodiment are compounds of formula (I-9k-1) which are compounds of formula (I-9k) wherein Q₁ is hydrogen, chlorine, cyclopropyl, 1,2,4-triazol-1-yl, triazol-1-yl or triazol-2-yl.

One further outstanding group of compounds according to this embodiment are compounds of formula (I-9k-2) which are compounds of formula (I-9k) wherein Q₁ is hydrogen, chlorine, cyclopropyl, or 1,2,4-triazol-1-yl.

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile, improved physico-chemical properties, or increased biodegradability or environmental profile). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

In another aspect the present invention provides a composition comprising an insecticidally, acaricidally, nematicidally or molluscicidally effective amount of a compound of formula (I), or an agrochemically acceptable salt, stereoisomer, enantiomer, tautomer or N-oxide thereof, as defined in any of the embodiments under compounds of formula (I-1), (I-2), (I-3), (I-4), (I-5), (I-6), (I-7), (I-8), and (I-9) (above), and, optionally, an auxiliary or diluent.

In a further aspect the present invention provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in any of the embodiments under compounds of formula (I-1), (I-2), (I-3), (I-4), (I-5), (I-6), (I-7), (I-8), and (I-9) (above) or a composition as defined above.

In a yet further aspect, the present invention provides a method for the protection of plant propagation material from the attack by insects, acarines, nematodes or molluscs, which comprises treating the propagation material or the site, where the propagation material is planted, with a composition as defined above.

The process according to the invention for preparing compounds of formula I is carried out in principle by methods known to those skilled in the art. More specifically, and as described in schemes 1 and 2, the subgroup of compounds of formula I, wherein X is SO (sulfoxide) and/or SO₂ (sulfone), may be obtained by means of an oxidation reaction of the corresponding sulfide compounds of formula I, wherein X is S, involving reagents such as, for example, m-chloroperoxybenzoic acid (mCPBA), hydrogen peroxide, oxone, sodium periodate, sodium hypochlorite or tert-butyl hypochlorite amongst other oxidants. The oxidation reaction is generally conducted in the presence of a solvent. Examples of the solvent to be used in the reaction include aliphatic halogenated hydrocarbons such as dichloromethane and chloroform; alcohols such as methanol and ethanol; acetic acid; water; and mixtures thereof. The amount of the oxidant to be used in the reaction is generally 1 to 3 moles, preferably 1 to 1.2 moles, relative to 1 mole of the sulfide compounds I to produce the sulfoxide compounds I, and preferably 2 to 2.2 moles of oxidant, relative to 1 mole of of the sulfide compounds I to produce the sulfone compounds I. Such oxidation reactions are disclosed, for example, in WO 2013/018928.

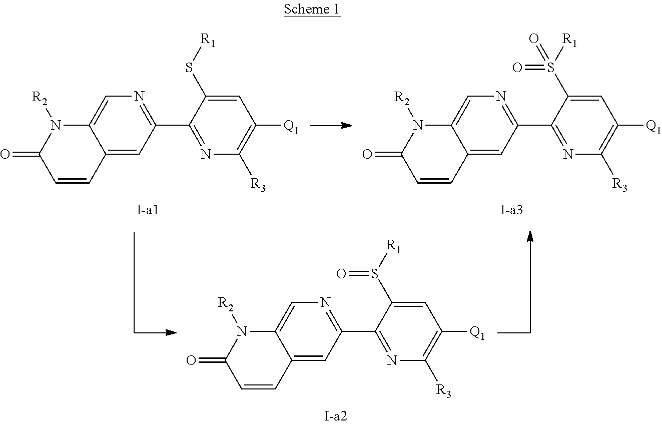

The chemistry described previously in scheme 1 to access compounds of formula I-a2 and I-a3 from compounds of formula I-a1 can be applied analogously (scheme 2) for the preparation of compounds of formula I-a5 and I-a6 from compounds of formula I-a4, wherein all substituent definitions mentioned previously remain valid.

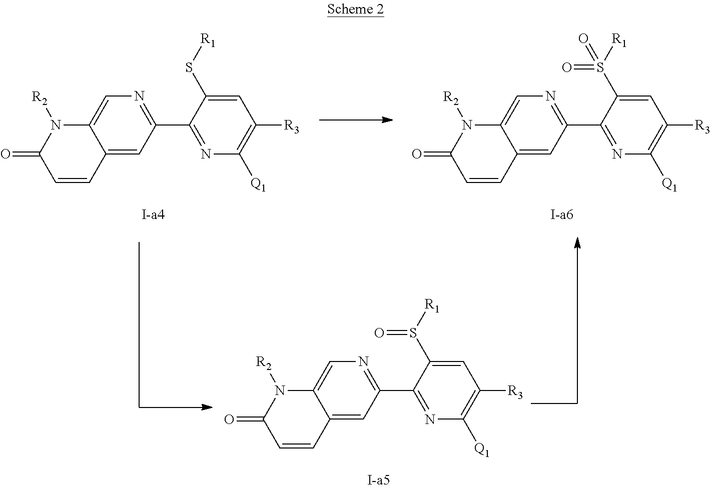

The subgroup of compounds of formula I, wherein R₂ is as defined in formula I and wherein Q is defined as Qa, in which A, Q₁, R₃, X and R₁ are as defined in formula I, may be defined as compounds of formula I-Qa (scheme 3).

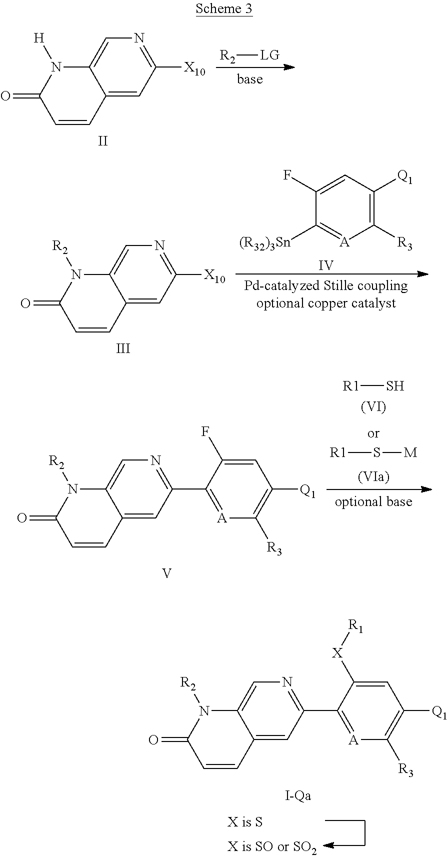

Compounds of formula I-Qa, wherein X is S, and in which A, R₁, R₂, Q₁, and R₃ are as defined in formula I, can be prepared by reacting compounds of formula V, wherein R₂, Q₁, A and R₃ are as defined in formula I, with a reagent of the formula VI

R₁—SH (VI),

or a salt thereof, wherein R₁ is as defined in formula I, optionally in the presence of a suitable base, such as alkali metal carbonates, for example sodium carbonate and potassium carbonate, or alkali metal hydrides such as sodium hydride, or alkali metal hydroxides such as sodium hydroxide and potassium hydroxide, or sodium or potassium tert-butoxide, in an inert solvent at temperatures preferably between 25-120° C. Examples of solvent to be used include ethers such as tetrahydrofuran THF, ethylene glycol dimethyl ether, tert-butylmethyl ether, and 1,4-dioxane, aromatic hydrocarbons such as toluene and xylene, nitriles such as acetonitrile or polar aprotic solvents such as N,N-dimethylformamide, N,N-dimethylacetamide, N-methyl-2-pyrrolidone NMP or dimethyl sulfoxide. Examples of salts of the compound of formula VI include compounds of the formula VIa

R₁—S-M (VIa),

wherein R₁ is as defined above and wherein M is, for example, sodium or potassium. Such a process to prepare compounds of formula I-Qa from compounds of formula V can be found, for example, in WO16/091731.

Alternatively, this reaction to form I-Qa can be carried out in the presence of a palladium catalyst, such as tris(dibenzylideneacetone)dipalladium(0), in the presence of a phosphine ligand, such as xanthphos, in an inert solvent, for example, xylene at temperatures between 100-160° C., preferably 140° C., as described in Tetrahedron 2005, 61, 5253-5259.

Compounds of formula V, wherein R₂, Q₁, A and R₃ are as defined in formula I, can be prepared by a Stille reaction between compounds of formula IV, wherein Q₁, A and R₃ are as defined in formula I above, and wherein R₃₂ is C₁-C₁₀alkyl, preferably n-butyl or methyl, and compounds of formula III, wherein R₂ is as defined in formula I above and wherein X₁₀ is a leaving group like, for example, chlorine, bromine or iodine (preferably chlorine or bromine), or an aryl- or alkylsulfonate such as trifluoromethanesulfonate. Such Stille reactions are usually carried out in the presence of a palladium catalyst, for example tetrakis(triphenylphosphine)palladium(0), palladium(II) acetate or bis(triphenylphosphine)palladium(II) dichloride, and in the presence of ligand such as phosphine ligand xanthphos, xphos amongst others in an inert solvent such as N,N-dimethylformamide, acetonitrile, toluene or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

Compounds of formula III, wherein R₂ is as defined in formula I above and wherein X₁₀ is a leaving group like, for example, chlorine, bromine or iodine (preferably chlorine or bromine), or an aryl- or alkylsulfonate such as trifluoromethanesulfonate are as defined in formula I, and in which X₁₀ is a halogen (or a pseudo-halogen leaving group, such as a triflate), preferably bromine or chlorine, can be prepared by reacting compounds of formula II, wherein X₁₀ is is a halogen (or a pseudo-halogen leaving group, such as a triflate), with reagents of the formula R₂-LG, wherein R₂ is as defined in formula I, and in which LG is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, in an inert solvent such as tetrahydrofuran, dioxane, N,N-dimethylformamide DMF, N,N-dimethylacetamide or acetonitrile and the like, at temperatures between 0 and 120° C., by procedures well known to those skilled in the art.

Such compounds of formula III

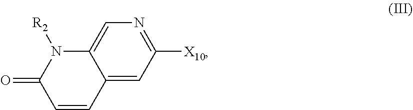

wherein

R₂ are as defined in formula I; and

X₁₀ is a halogen or a pseudo-halogen leaving group, such as a triflate, are novel, especially developed for the preparation of the compounds of formula I according to the invention and therefore represent a further object of the invention. The preferences and preferred embodiments of the substituents of the compounds of formula I are also valid for the compounds of formula III. Preferably, X₁₀ is bromo or chloro; even more preferably X₁₀ is bromo.

The chemistry described previously in scheme 3 to access compounds of formula I-Qa from compounds of formula II can be applied analogously (scheme 4) for the preparation of compounds of formula I-Qb from compounds of formula II, wherein all substituent definitions mentioned previously remain valid.

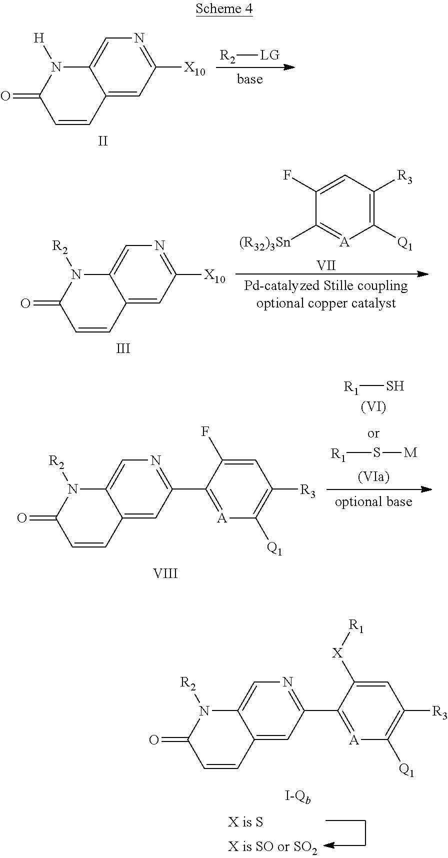

Compounds of formula VI, wherein R₁ is as defined in formula I, and compounds of formula VIa, wherein R₁ is as defined above and wherein M is, for example, sodium or potassium, are either known, commercially available or may be prepared by methods known to a person skilled in the art.

Compounds of formula IV and compounds of formula VII, wherein Q₁, A and R₃ are as defined in formula I above, and wherein R₃₂ is C₁-C₁₀alkyl, preferably n-butyl or methyl; and reagents of the formula R₂-LG, wherein R₂ is as defined in formula I, and in which LG is a halogen, preferably iodine, bromine or chlorine (or a pseudo-halogen leaving group, such as a (halo)alkyl or phenyl sulfonate ester, e.g. triflate);

are either known, commercially available or may be prepared by methods known to a person skilled in the art.

Alternatively compounds of formula I-Qa, wherein R₁, R₂, R₃, Q₁ and X are as defined in formula I above, and in which A is N, can be prepared following scheme 5.

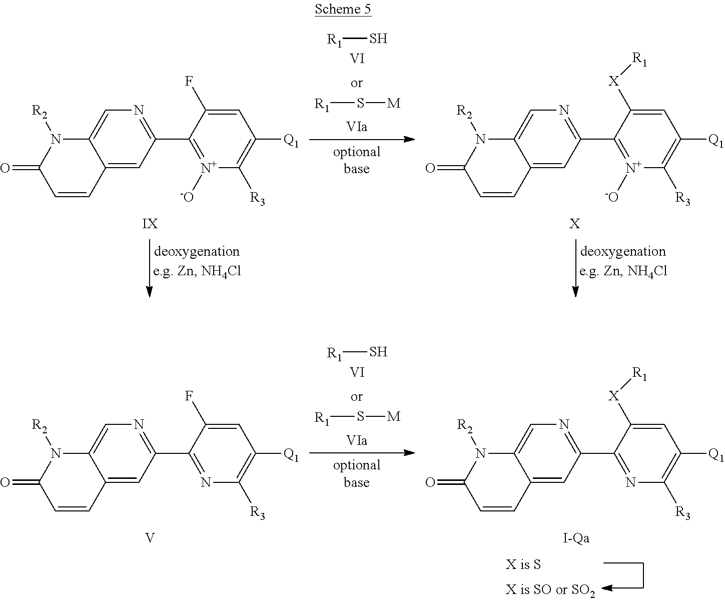

Compounds of formula I-Qa, wherein X is S, and in which R₁, R₂, R₃, and Q₁ are as defined in formula I above are prepared (scheme 5) by deoxygenation of compounds of formula X, wherein X is S, and in which R₁, R₂, Q₁, and R₃ are as defined in formula I, using reagents such as zinc powder and ammonium chloride, preferably an aqueous saturated ammonium chloride solution, in ether solvents such as tetrahydrofuran or dioxane, at temperatures between 0° C. and refluxing conditions. Alternatively, such a reduction may also be achieved under conditions known to a person skilled in the art, for example by involving iron powder in acetic acid, or using molecular hydrogen (H₂), optionally under pressure, usually in the presence of a catalyst such as for example Raney-Nickel, or using transfer hydrogenation conditions (for example, ammonium formate and 5-10% palladium on charcoal in tetrahydrofuran around room temperature), or using bis(pinacolato)diboron (4,4,5,5-tetramethyl-2-(4,4,5,5-tetramethyl-1,3,2-dioxaborolan-2-yl)-1,3,2-dioxaborolane), or using phosphorus based reagents such as phosphorus trichloride, triethyl phosphite or triphenyl phosphine.

Compounds of formula X, wherein X is S, and in which R₁, R₂, Q₁, and R₃ are as defined in formula I, can be prepared from compounds of formula IX, wherein R₂, R₃ and Q₁ are as described in formula I above, by analogues procedure as described in scheme 3 for the preparation of compounds of formula I-Qa from compounds of formula V.

Such compounds of formula X

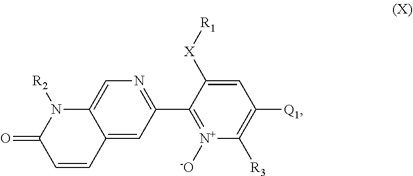

wherein

X is S, and in which R₂, Q₁, R₃ and R₁ are as defined in formula I, are novel, especially developed for the preparation of the compounds of formula I according to the invention and therefore represent a further object of the invention. The preferences and preferred embodiments of the substituents of the compounds of formula I are also valid for the compounds of formula X.

Alternatively, compounds of formula I-Qa, wherein X is S, may be prepared from compounds of formula IX, by involving the same chemistry as described above, but by changing the order of the steps (i.e. by running the sequence IX to V via deoxygenation/reduction, followed by reaction of V with VI or VIa to form I-Qa, wherein all substituent definitions mentioned previously remain valid).

Compounds of formula V

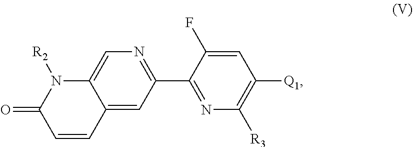

wherein

R₂, Q₁ and R₃ are as defined in formula I,

are novel, especially developed for the preparation of the compounds of formula I according to the invention and therefore represent a further object of the invention. The preferences and preferred embodiments of the substituents of the compounds of formula I are also valid for the compounds of formula V.

The chemistry described previously in scheme 5 to access compounds of formula I-Qa from compounds of formula IX can be applied analogously (scheme 6) for the preparation of compounds of formula I-Qb from compounds of formula XI, wherein all substituent definitions mentioned previously remain valid.

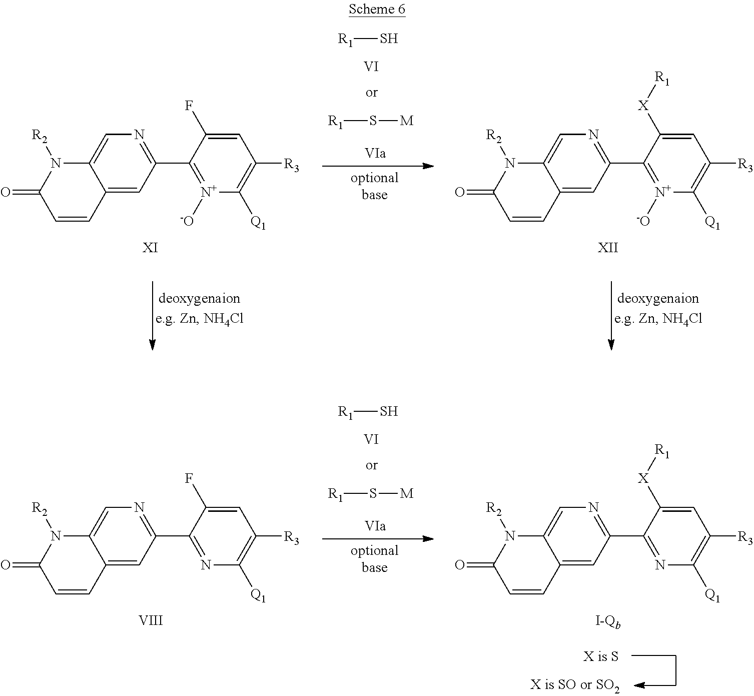

Compounds of formula IX, wherein R₂, Q₁ and R₃ are as defined in formula I,

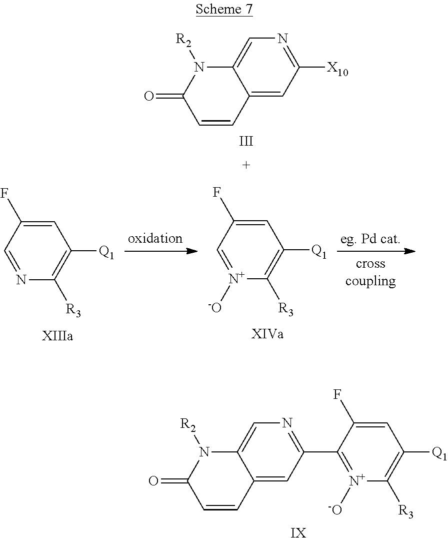

can be prepared (scheme 7) by a cross-coupling reaction between compounds of formula III, wherein R₂ is as defined in formula I above and wherein X₁₀ is is a halogen (or a pseudo-halogen leaving group, such as a triflate), preferably bromine or chlorine, and compounds of formula XIVa, wherein Q₁ and R₃ are as defined in formula I, under metal catalysis (preferably palladium catalysis) conditions, for example involving [1,1′bis(diphenylphosphino)ferrocene]dichloropalladium (PdCl₂(dppf), optionally as a complex with dichloromethane (preferably a 1:1 complex), in presence of a base such as 2,2,6,6-tetramethylpiperidide zinc chloride lithium chloride (TMPZnC.LiCl; commercial or prepared according to Org. Lett. 2009, 11, 1837-1840), preferably in form of a solution of the tetramethylpiperidinyl zinc chloride lithium chloride complex in tetrahydrofuran, in ether solvents such as tetrahydrofuran, dioxane or 1,2-dimethoxyethane, preferably tetrahydrofuran, at temperatures between 0° C. and refluxing conditions, preferably between room temperature and 80° C., preferably under inert atmosphere, and optionally microwave irradiation. Such cross-coupling conditions have been described in, for example, Org. Lett. 2012, 14, 862-865.

Alternatively, this cross-coupling step may also perform under Fagnou-type conditions (described by Fagnou et al. in, for example, Org. Lett. 2011, 13, 2310-13 and J. Am. Chem. Soc. 2009, 131, 3291-3306) involving palladium acetate and a phosphine ligand such as tri-tert-butylphosphonium tetrafluoroborate (PtBu₃-HBF₄), in the presence of a base such as potassium carbonate or cesium carbonate, in solvents such as tetrahydrofuran, dioxane, acetonitrile, N,N-dimethylformamide or toluene, at temperatures between 0° C. and 150° C., preferably between room temperature and 120° C., preferably under inert atmosphere, and optionally microwave irradiation.

Such compounds of formula IX

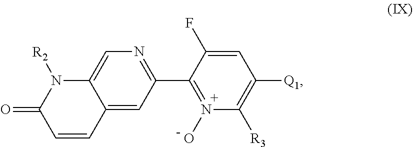

wherein

R₂, Q₁ and R₃ are as defined in formula I, are novel, especially developed for the preparation of the compounds of formula I according to the invention and therefore represent a further object of the invention. The preferences and preferred embodiments of the substituents of the compounds of formula I are also valid for the compounds of formula IX.

Compounds of formula XIVa, wherein Q₁ and R₃ are as defined in formula I, can be prepared by oxidation of compounds of formula XIIIa, wherein Q₁ and R₃ are as defined in formula I, under conditions known to those skilled in the art, involving for example, meta-chloro perbenzoic acid in an inert solvent such as ethyl acetate, chloroform or methylene chloride, at temperatures between 0° C. and 80° C., preferably 10 to 70° C. Alternatively, other suitable oxidizing agents may be used, such as for example methyltrioxorhenium and hydrogen peroxide (either aqueous or as a urea complex), hydrogen peroxide in acetic acid, or the H₂O₂/urea adduct in the presence of an acid anhydride, e.g. trifluoroacetic anhydride. Such oxidations are known from the literature, for example from J. Med. Chem. 1989, 32, 2561, WO 00/15615 or WO 20/182577.

Compounds of formula XIIIa, wherein Q₁ and R₃ are as defined in formula I, are either known, commercially available, or may be prepared by methods known to a person skilled in the art or by analogy to descriptions found for example in WO 20/182577.

The chemistry described previously in scheme 7 to access compounds of formula IX can be applied analogously (scheme 8) for the preparation of compounds of formula XI, wherein all substituent definitions mentioned previously remain valid.

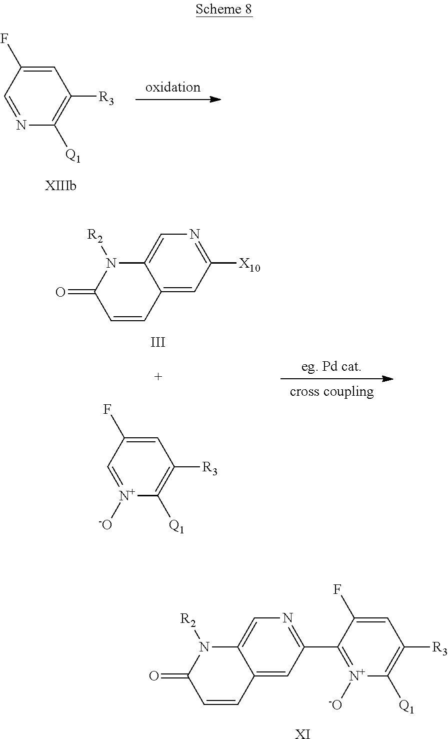

Compounds of formula XIIIb, wherein Q₁ and R₃ are as defined in formula I, are either known, commercially available, or may be prepared by methods known to a person skilled in the art.

Compounds of formula II can be prepared following scheme 9.

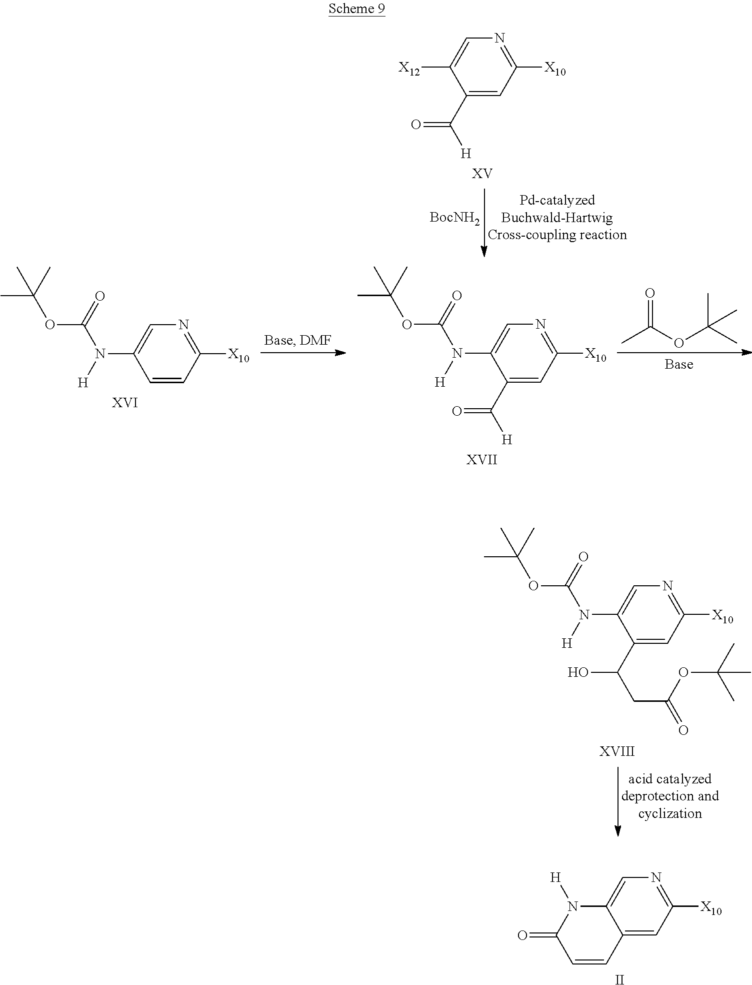

Compounds of formula II, wherein X₁₀ is a halogen (or a pseudo-halogen leaving group, such as a triflate), preferably bromine or chlorine, can be prepared (scheme 9) by acid catalyzed deprotection of BOC-functional groups (tert-butoxycarbonyl) and subsequent intramolecular cyclization of amine and carboxylic acid to form the carboxamide. Such reactions can be performed in the presence of acids such as trifluoroacetic acid, hydrochloric acid, sulfuric acid amongst others and optionally in the presence of solvents such as halogenated solvents like dichloromethane, dichloroethane, water amongst others and at temperature between room temperature and boiling point of the solvent or reagent. Compounds of formula XVIII, wherein X₁₀ is a halogen (or a pseudo-halogen leaving group, such as a triflate), preferably bromine or chlorine, can be prepared by the reaction of compounds of formula XVII, wherein X₁₀ is a halogen (or a pseudo-halogen leaving group, such as a triflate), preferably bromine or chlorine, and tert-butyl acetate in the presence of a suitable base such as n-BuLi, lithium diisopropylamide, Li-TMP amongst others and in the presence of solvent such as tetrahydrofuran, dioxane, dimethylformamide amongst other and at temperatures between −78° C. and boiling point of solvent. Compounds of formula XVII, wherein X₁₀ is a halogen (or a pseudo-halogen leaving group, such as a triflate), preferably bromine or chlorine, can be prepared by the metallation of compounds of formula XVI, wherein X₁₀ is a halogen (or a pseudo-halogen leaving group, such as a triflate), preferably bromine or chlorine, using base such as n-BuLi, lithium diisopropylamide, Li-TMP and subsequently reacting with DMF in the presence of solvent such as tetrahydrofuran, dioxane, dimethylformamide. Such reactions are known for example in J. Org Chem., 1990, 55, 4744. Alternatively compounds of formula XVII, can be prepared by palladium catalyzed selective Buchwald-Hartwig cross-coupling reaction between compounds of formula XV, wherein X₁₀ and X₁₂ are independently halogen (or a pseudo-halogen leaving group, such as a triflate), preferably bromine or chlorine and tert-butyl carbamate (BOCNH₂). Such reactions can be performed in the presence of metal catalyst (preferably palladium catalyst) such as [1,1′bis(diphenylphosphino)ferrocene]-dichloropalladium (PdCl₂(dppf), or Pd(OAc)₂ and in the presence of ligand such as tributylphosphine, dppf, Xantphos, Xphos and in presence of a base such as sodium tert-butoxide, potassium carbonate, cesium carbonate, sodium carbonate and in the presence of solvents such as tetrahydrofuran, dioxane or 1,2-dimethoxyethane, toluene, and at temperatures between 0° C. and refluxing conditions, preferably under inert atmosphere, and optionally microwave irradiation. Such reactions are well known in the literature and described for example in Chem. Rev. 2016, 116, 19, 12564-12649 and J. Org. Chem. 1999, 64, 15, 5575-5580.

Alternatively compounds of formula II, wherein X₁₀ is a halogen (or a pseudo-halogen leaving group, such as a triflate), preferably bromine or chlorine,

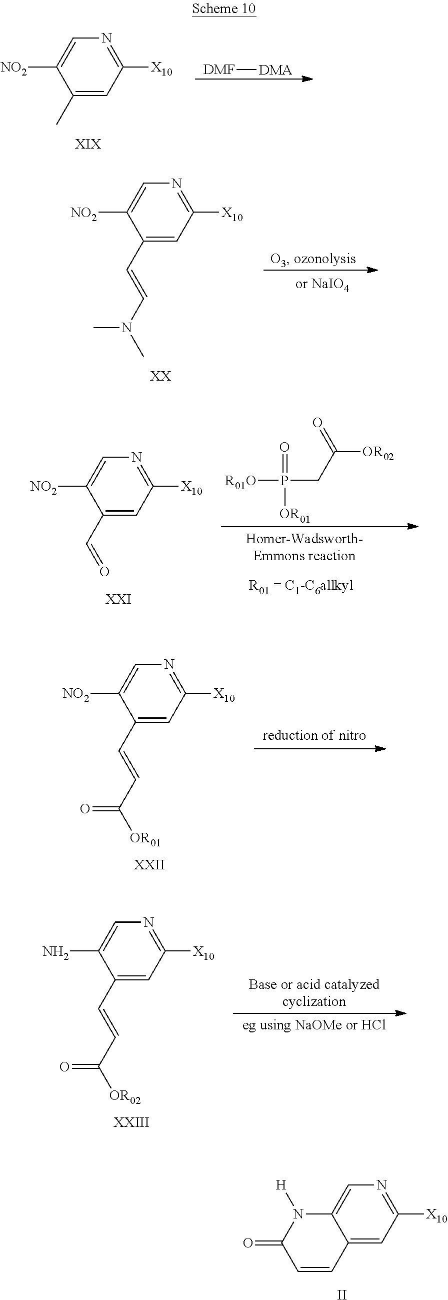

can be prepared following scheme 10 and analogous to procedures as described in WO 2000/049015.

Alternatively compounds of formula II, wherein X₁₀ is a halogen (or a pseudo-halogen leaving group, such as a triflate), preferably bromine or chlorine,

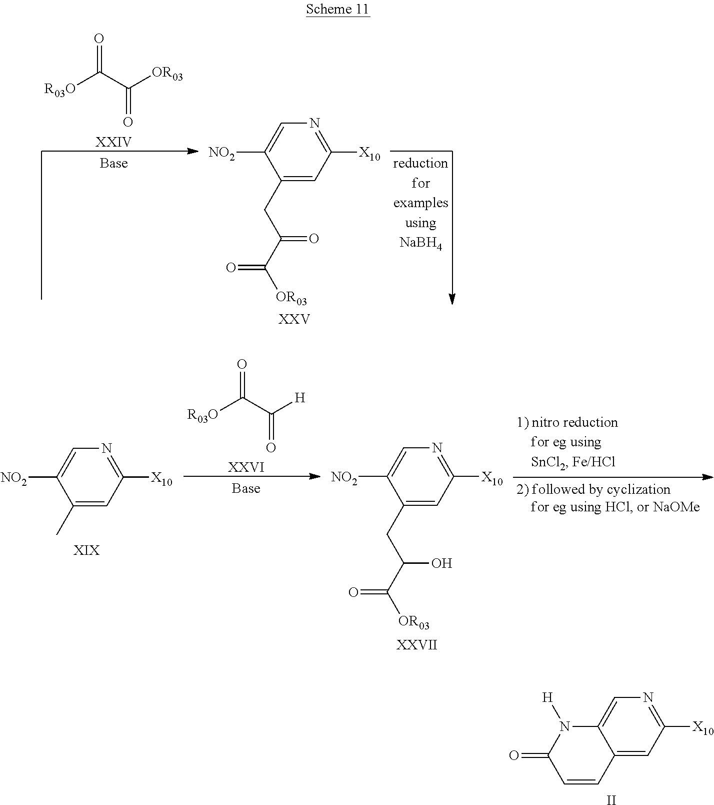

can be prepared (scheme 11) from compounds of formula XXVII, wherein X₁₀ is a halogen (or a pseudo-halogen leaving group, such as a triflate) preferably bromine or chlorine, and in which R₀₃ is C₁-C₆alkyl, benzyl, or an aryl group, via nitro reduction and subsequent intramolecular cyclization in the presence of an acid catalyst, such as acetic acid, hydrochloric acid HCl, sulfuric acid H₂SO₄, or trifluoroacetic acid TFA, or in the presence of a base, such as sodium methoxide.

Nitro reduction typically make use of reagents such as iron in the presence of ammonium chloride, Sn/HCl, or tetrahydroxydiboron amongst others, and at temperature between 0° C. to boiling point of the reaction mixture. Such reactions are known in the literature and for example described for example in Synthesis, 2018, 50, 1765-1768, Org. Lett. 2014, 16, 19, 5192-5195, Organic Letters (2019), 21(9), 3465-3469 and Synthetic Communications, 2007, 37, 2777-2786.

Compounds of formula XXV may exist in different tautomeric forms, such as XXVa and/or XXVb:

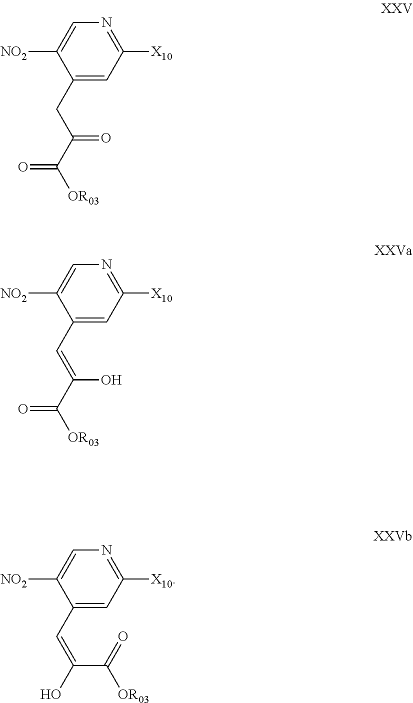

This invention covers all such isomers and tautomers and mixtures thereof in all proportions.

Compounds of formula XXVII, wherein X₁₀ is a halogen (or a pseudo-halogen leaving group, such as a triflate), preferably bromine or chlorine, and in which R₀₃ is C₁-C₆alkyl, benzyl, or an aryl group, can be prepared by reacting compounds of formula XIX, wherein X₁₀ is a halogen (or a pseudo-halogen leaving group, such as a triflate), preferably bromine or chlorine, and compounds of formula XXVI, wherein R₀₃ is C₁-C₆alkyl, benzyl, or an aryl group, in the presence of base such as potassium tert-butoxide, sodium methoxide, sodium ethoxide, sodium hydride, n-butyl lithium, 1,8-diazabicyclo(5.4.0)undec-7-ene (DBU), lithium diisopropylamide, amongst other similar bases, optionally in the presence of a solvent such as tetrahydrofuran, methanol, dioxane, ethanol, DMF and at temperature in between −78° C. to the boiling point of the reaction mixture.

Alternatively compounds of formula XXVII, wherein X₁₀ is a halogen (or a pseudo-halogen leaving group, such as a triflate), preferably bromine or chlorine, and in which R₀₃ is C₁-C₆alkyl, benzyl, or an aryl group, can be prepared in two steps from compounds of formula XIX. The first step involves reacting compounds of formula XIX and compounds of formula XXIV, wherein R₀₃ is C₁-C₆alkyl, benzyl, or an aryl group, in the presence of base such as potassium tert-butoxide, sodium methoxide, sodium ethoxide, sodium hydride, n-butyl lithium, 1,8-diazabicyclo(5.4.0)undec-7-ene (DBU), lithium diisopropylamide, amongst other similar bases, optionally in the presence of solvent such as tetrahydrofuran, methanol, dioxane, ethanol, DMF and at temperature in between −78° C. to the boiling point of the reaction mixture. And the second step involves the carbonyl reduction of compounds of formula XXV, wherein X₁₀ is a halogen (or a pseudo-halogen leaving group, such as a triflate), preferably bromine or chlorine, and in which R₀₃ is C₁-C₆alkyl, benzyl, or an aryl group, in the presence of reducing agent such as a boron based reducing agent, for example sodium borohydride, borane, or an aluminum based reagent, for example diisobutylaluminium hydride, or lithium aluminum hydride. Such two step procedures are known in the literature, for example as described in WO2005/044802.

Compounds of formula XV, wherein X₁₀ and X₁₂ are independently halogen (or a pseudo-halogen leaving group, such as a triflate), preferably bromine or chlorine; and

compounds of formula XVI, wherein X₁₀ is a halogen (or a pseudo-halogen leaving group, such as a triflate), preferably bromine or chlorine; and

compounds of formula XIX, wherein X₁₀ is a halogen (or a pseudo-halogen leaving group, such as a triflate), preferably bromine or chlorine; and

compounds of formula XXIV and XXVI, wherein R₀₃ is C₁-C₆alkyl, benzyl, or an aryl group; are either known, commercially available, or may be prepared by methods known to a person skilled in the art.

Alternatively compounds of formula I-Qb can also be prepared following scheme 12. In the particular situation when Q₁ is an optionally substituted triazole linked via a nitrogen atom to the ring which contains the group A, then compounds of formula I-Qb, wherein X is S and in which R₁, R₂, Q₁ and R₃ are as defined in formula I,

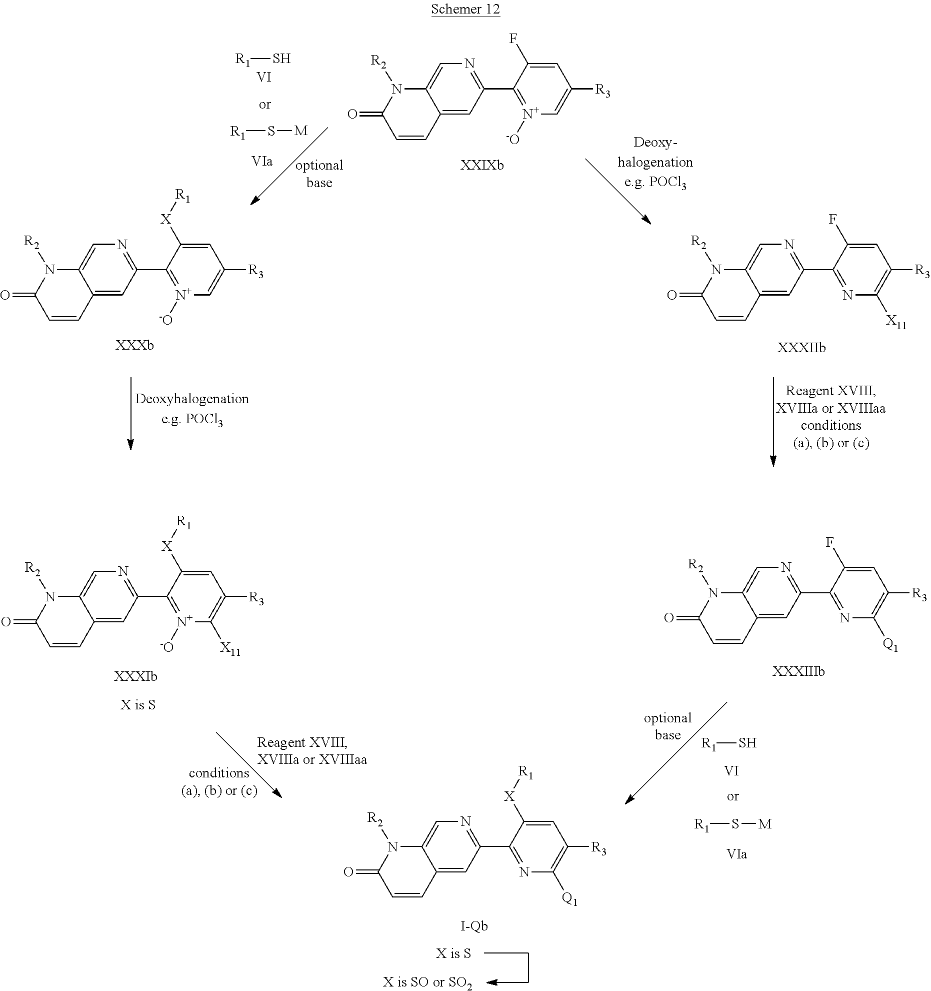

may alternatively be prepared (scheme 12) from compounds of formula XXXIb, wherein X is S and in which R₁, R₂, Q₁ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with an optionally substituted triazole Q₁-H (which contains an appropriate NH functionality) (XVIIIaa), wherein Q₁ is N-linked triazolyl, in solvents such as alcohols (eg. methanol, ethanol, isopropanol, or higher boiling linear or branched alcohols), pyridine or acetic acid, optionally in the presence of an additional base, such as potassium carbonate K₂CO₃ or cesium carbonate Cs₂CO₃, optionally in the presence of a copper catalyst, for example copper(I) iodide, at temperatures between 30-180° C., optionally under microwave irradiation.

In the particular situation within scheme 12 when Q₁ is —N(R₄)COR₅, or —N(R₄)CON(R₄)₂, wherein R₄ and R₅ are as defined in formula I, then compounds of formula I-Qb, wherein X is S, may be prepared from compounds of formula XXXIb, wherein X is S and in which R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with a reagent Q₁-H (XVIIIaa) equivalent to HN(R₄)COR₅, or HN(R₄)CON(R₄)₂, wherein R₄ and R₅ are as defined in formula I. Such a reaction is performed in the presence of a base, such as potassium carbonate, cesium carbonate, sodium hydroxide, in an inert solvent, such as toluene, dimethylformamide DMF, N-methyl pyrrolidine NMP, dimethyl sulfoxide DMSO, dioxane, tetrahydrofuran THF, and the like, optionally in the presence of a catalyst, for example palladium(II)acetate, bis(dibenzylideneacetone)palladium(0) (Pd(dba)₂) or tris(dibenzylideneacetone) dipalladium(0) (Pd₂(dba)₃, optionally in form of a chloroform adduct), or a palladium pre-catalyst such as for example tert-BuBrettPhos Pd G3 [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate or BrettPhos Pd G3 [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate, and optionally in the presence of a ligand, for example SPhos, t-BuBrettPhos or Xantphos, at temperatures between 60-120° C., optionally under microwave irradiation.

In the particular situation within scheme 12 when Q₁ is —N(R₄)₂, wherein R₄ is as defined in formula I, then compounds of formula I-Qb, wherein X is S, may be prepared from compounds of formula XXXIb, wherein X is S and in which R₁, R₂ and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with a reagent Q₁-H (XVIIIaa) equivalent to HN(R₄)₂, or a salt thereof (such as a hydrohalide salt, preferably a hydrochloride or a hydrobromide salt, or a trifluoroacetic acid salt, or any other equivalent salt), wherein R₄ is as defined in formula I. Such a reaction is commonly performed in an inert solvent such as alcohols, amides, esters, ethers, nitriles and water, particularly preferred are methanol, ethanol, 2,2,2-trifluoroethanol, propanol, isopropanol, N,N-dimethylformamide, N,N-dimethylacetamide, dioxane, tetrahydrofuran, dimethoxyethane, acetonitrile, ethyl acetate, toluene, water or mixtures thereof, at temperatures between 0-150° C., optionally under microwave irradiation or pressurized conditions using an autoclave, optionally in the presence of a copper catalyst, such as copper powder, copper(I) iodide or copper sulfate (optionally in form of a hydrate), or mixtures thereof, optionally in presence a ligand, for example diamine ligands (e.g. N,N′-dimethylethylenediamine or trans-cyclohexyldiamine) ordibenzylideneacetone (dba), or 1,10-phenanthroline, and optionally in presence of a base such as potassium phosphate.

Reagents HN(R₄)₂, HN(R₄)COR₅, or HN(R₄)CON(R₄)₂, wherein R₄ and R₅ are as defined in formula I, are either known, commercially available or may be prepared by methods known to a person skilled in the art.

Alternatively, compounds of formula I-Qb, wherein X is S, may be prepared by a Suzuki reaction (scheme 12), which involves for example, reacting compounds of formula XXXIb, wherein X is S and in which R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, with compounds of formula XVIII, wherein Q₁ is as defined in formula I, and wherein Y_{b1} can be a boron-derived functional group, such as for example B(OH)₂ or B(OR_{b1})₂ wherein R_{b1} can be a C₁-C₄alkyl group or the two groups OR_{b1} can form together with the boron atom a five membered ring, as for example a pinacol boronic ester. The reaction may be catalyzed by a palladium based catalyst, for example tetrakis(triphenyl-phosphine)palladium(0), (1,1′bis(diphenylphosphino) ferrocene)dichloro-palladium-dichloromethane (1:1 complex) or chloro(2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethylformamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art and have been reviewed, for example, in J. Orgmet. Chem. 576, 1999, 147-168.

Alternatively compounds of formula I-Qb, wherein X is S, may be prepared by a Stille reaction between compounds of formula XVIIIa, wherein Q₁ is as defined above, and wherein Y_{b2} is a trialkyl tin derivative, preferably tri-n-butyl tin or tri-methyl-tin, and compounds of formula XXXIb, wherein X is S and in which R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate. Such Stille reactions are usually carried out in the presence of a palladium catalyst, for example tetrakis(triphenylphosphine)palladium(0), or bis(triphenylphosphine) palladium(II) dichloride, in an inert solvent such as N,N-dimethylformamide, acetonitrile, toluene or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

When Q₁ is a five-membered aromatic ring system linked via a nitrogen atom to the ring which contains the substituent A, then compounds of formula I-Qb, wherein X is S, may be prepared from compounds of formula XXXIb, wherein X is S and in which R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction with a heterocycle Q₁-H (which contains an appropriate NH functionality) (XVIIIaa), wherein Q₁ is as defined above, in the presence of a base, such as potassium carbonate K₂CO₃ or cesium carbonate Cs₂CO₃, optionally in the presence of a copper catalyst, for example copper(I) iodide, with or without an additive such as L-proline, N,N′-dimethylcyclohexane-1,2-diamine or N,N′-dimethyl-ethylene-diamine, in an inert solvent such as N-methylpyrrolidone NMP or N,N-dimethylformamide DMF at temperatures between 30-150° C., optionally under microwave irradiation.

A large number of compounds of the formula (XVIII), (XVIIIa) and (XVIIIaa) are commercially available or can be prepared by those skilled in the art.

Alternatively, compounds of formula I-Qb, wherein X is SO or SO₂, may be prepared from compounds of formula XXXIb, wherein X is SO or SO₂ and in which R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, by involving the same chemistry as described above, but by changing the order of the steps (i.e. by running an oxidation step on XXXIb, wherein X is S, to form XXXIb, wherein X is SO or SO₂, followed by the sequence XXXIb (X is SO or SO₂) to XXXIb (X is SO or SO₂) via Suzuki, Stille or C—N bond formation).

Oxidation of compounds of formula XXXIb, wherein X is S and in which R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, with a suitable oxidizing agent, into compounds of formula XXXIb, wherein X is SO or SO₂ may be achieved under conditions already described above.

Compounds of formula XXXIb, wherein X is S and in which R₁, R₂, and R₃ are as defined in formula I, and wherein X₁₁ is a leaving group like, for example, chlorine, bromine or iodine (preferably chlorine or bromine), or an aryl- or alkylsulfonate such as trifluoromethanesulfonate, may be prepared (scheme 12) by reacting compounds of formula XXXb, wherein X is S and in which R₁, R₂, and R₃ are as defined in formula I, with a halogenating agent such as phosphorus oxychloride POCl₃ or phosphorus oxybromide, neat or in an appropriate solvent, such as chloroform or toluene, optionally in the presence of a base, such as triethylamine or pyridine, at temperatures between room temperature and refluxing conditions. Such deoxyhalogenation have been described in, for example, WO16/116338.

Compounds of Formula XXXIb-1

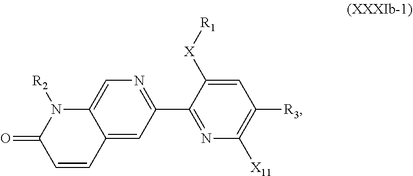

wherein

X is S, SO or SO₂;

X₁₁ is a halogen or a pseudo-halogen leaving group; and

R₁, R₂ and R₃ are as defined under formula I in claim 1,

are novel, especially developed for the preparation of the compounds of formula I according to the invention and therefore represent a further object of the invention. The preferences and preferred embodiments of the substituents of the compounds of formula I are also valid for the compounds of formula XXXIb-1. Preferably, X₁₁ is bromo or chloro; even more preferably X₁₁ is chloro.

Compounds of formula XXXb, wherein X is S and in which R₁, R₂, and R₃ are as defined in formula I, may be prepared by reacting compounds of formula XXIXb, wherein R₂ and R₃ are as defined in formula I, with reagents of formula VI or VIa, wherein R₁ is as defined in formula I, under conditions already described above (see text scheme 3).

Compounds of formula XXIXb, wherein R₂ and R₃ are as defined in formula I, may be prepared by cross-coupling compounds of formula III, wherein R₂ is as defined in formula I above and wherein X₁₀ is is a halogen (or a pseudo-halogen leaving group, such as a triflate), preferably bromine or chlorine, with compounds of formula XIVb-1, wherein R₃ is as defined in formula I, as described in scheme 13 and under conditions already described above (see text schemes 7 and 8).

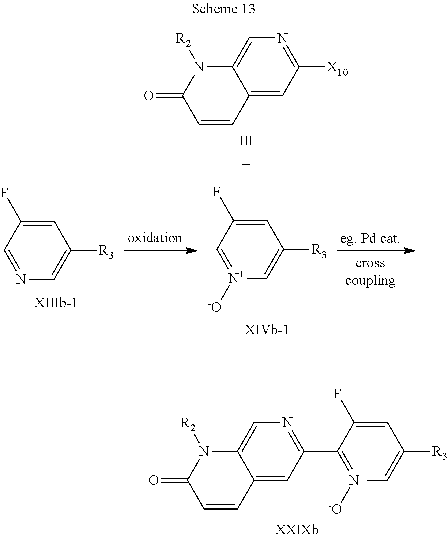

Compounds of formula XIIIb-1, wherein R₃ is as defined in formula I, are oxidized by methods described above (see text schemes 7 and 8) into compounds of formula XIVb-1, wherein R₃ is as defined in formula I.

Compounds of formula XIIIb-1, wherein R₃ is as defined in formula I, are either known, commercially available or may be prepared by methods known to a person skilled in the art.

Alternatively, compounds of formula I-Qb, wherein X is S, SO or SO₂, may be prepared (scheme 12) from compounds of formula XXIXb, by involving the same chemistry as described above, but by changing the order of the steps (i.e. by running the sequence XXIXb to XXXIIb, XXXIIb to XXXIIIb which was described previously, and XXXIIIb to I-Qb, followed by oxidation, and wherein all substituent definitions mentioned previously remain valid).

In the particular situation when R₃ is C₁-C₄alkyl, then compounds of formula I-Qa, wherein X is S and in which R₁, R₂, Q₁ and R₃ are as defined in formula I,

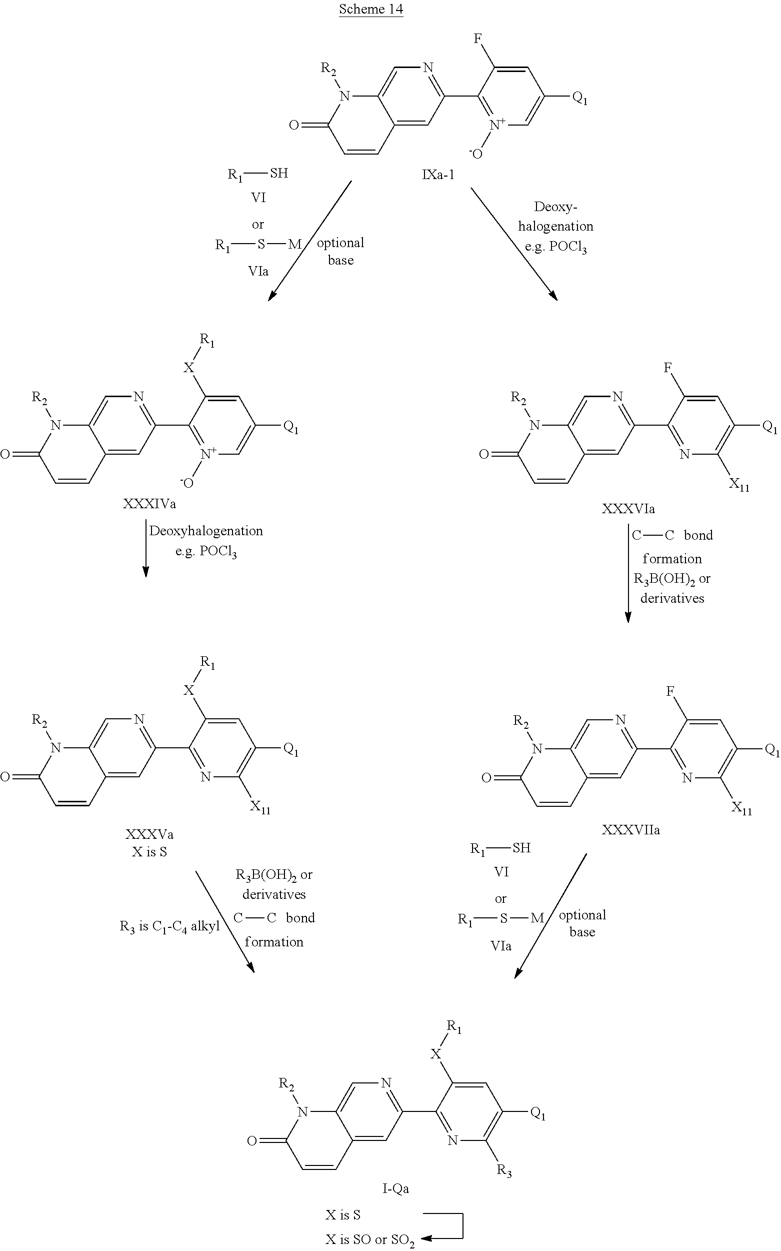

may alternatively be prepared (scheme 14) from compounds of formula XXXVa, wherein X is S and in which R₁, Q₁ and R₂ are as defined in formula I, and wherein X₁₁ is a leaving group like, for example, chlorine, bromine or iodine (preferably chlorine or bromine), or an aryl- or alkylsulfonate such as trifluoromethanesulfonate, by means of a C—C bond formation reaction typically under palladium-catalyzed (alternatively nickel-catalyzed) cross-coupling conditions. Such Suzuki-Miyaura cross-coupling reactions between compounds of formula XXXVa and C₁-C₄alkyl boronic acids of the formula R₃B(OH)₂ wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, are well known to a person skilled in the art. In the particular situation where R₃ is methyl, compounds of formula XXXVa can be reacted, for example, with trimethylboroxine (also known as 2,4,6-trimethyl-1,3,5,2,4,6-trioxatriborinane) in the presence of palladium catalyst, such as tetrakis(triphenylphosphine)-palladium(0) or [1,1′-bis(diphenylphosphino)ferrocene]palladium(II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such conditions are described, for example, in Tetrahedron Letters (2000), 41(32), 6237-6240.

Compounds of formula XXXVa, wherein X is S and in which R₁, R₂, and Q₁ are as defined in formula I, and wherein X₁₁ is a leaving group like, for example, chlorine, bromine or iodine (preferably chlorine or bromine), or an aryl- or alkylsulfonate such as trifluoromethanesulfonate, may be prepared from compounds of formula IXa-1 (via compounds of formula XXXIVa), wherein Q₁ is as defined in formula I, in a sequence and under conditions already described above (see text scheme 12), and wherein all substituent definitions mentioned previously remain valid).

Alternatively, compounds of formula I-Qa, wherein X is SO or SO₂, may be prepared from compounds of formula XXXVa, wherein X is SO or SO₂ and in which R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoromethanesulfonate, by involving the same chemistry as described above, but by changing the order of the steps (i.e. by running an oxidation step on XXXVa, wherein X is S, to form XXXVa, wherein X is SO or SO₂, followed by the sequence XXXVa (X is SO or SO₂) to I-Qa (X is SO or SO₂) via C—C bond formation with R₃B(OH)₂, or equivalent).

Oxidation of compounds of formula XXXVa, wherein X is S and in which R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoromethanesulfonate, with a suitable oxidizing agent, into compounds of formula XXXVa, wherein X is SO or SO₂ may be achieved under conditions already described above.

Alternatively, compounds of formula I-Qa, wherein X is S, SO or SO₂, may be prepared (scheme 14) from compounds of formula IXa-1, by involving the same chemistry as just described above, but by changing the order of the steps (i.e. by running the sequence IXa-1 to XXXVIa, XXXVIa to XXXVIIa which was described previously, and XXXVIIa to I-Qa, followed by oxidation, and wherein all substituent definitions mentioned previously remain valid).

In the particular situation when R₃ is hydrogen, then compounds of formula I-Qa, wherein X is S, SO or SO₂, and in which R₁, R₂ and Q₁ are as defined in formula I, may alternatively be prepared (scheme 14) from compounds of formula XXXVa, wherein X is S, SO or SO₂, and in which R₁, R₂ and Q₁ are as defined in formula I, and wherein X₁₁ is a leaving group like, for example, chlorine, bromine or iodine (preferably chlorine or bromine), or an aryl- or alkylsulfonate such as trifluoro-methanesulfonate, by means of a reductive dehalogenation. Such a hydrodehalogenation can be achieved, for example, using zinc dust and acetic acid or trifluoroacetic acid, or mixtures thereof, at temperatures between 0° C. and 120° C., preferably between 50° C. and reflux temperature, as described, for example, in Journal of the Chemical Society, Perkin Transactions 1: Organic and Bio-Organic Chemistry (1972-1999), (10), 2501-6, 1983 or in US20100076027.

C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, are either known, commercially available or may be prepared by methods known to a person skilled in the art.

Compounds of formula IXa-1, wherein R₂ and Q₁ are as defined in formula I, may be prepared by cross-coupling compounds of formula III, wherein R₂ is as defined in formula I above and wherein X₁₀ is is a halogen (or a pseudo-halogen leaving group, such as a triflate), preferably bromine or chlorine, with compounds of formula XIVa-1, wherein Q₁ is as defined in formula I, as described in scheme 15 and under conditions already described above (see text schemes 7 and 8).

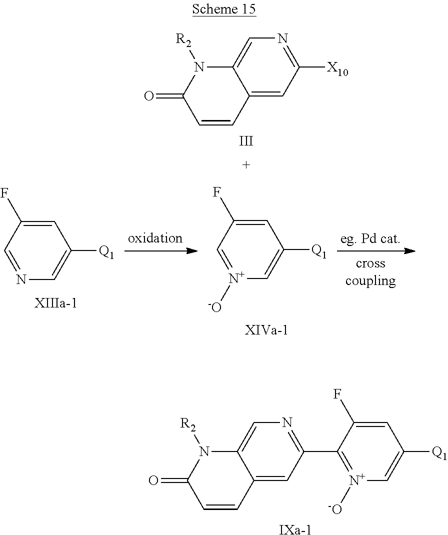

Compounds of formula XIIIa-1, wherein Q₁ is as defined in formula I, are oxidized by methods described above (see text schemes 7 and 8) into compounds of formula XIVa-1, wherein Q₁ is as defined in formula I.

Compounds of formula XIIIa-1 and compounds of formula XIVa-1, wherein Q₁ is as defined in formula I, are either known, commercially available, or may be prepared by methods known to a person skilled in the art or by analogy to descriptions found for example in WO 20/182577.

The subgroup of compounds of formula I, wherein R₂ is as defined in formula I and wherein Q is defined as Qa, in which A is N and R₃, X and R₁ are as defined in formula I, and wherein Q₁ is —N(R₄)COR₅, in which R₄ and R₅ are as defined in formula I, may be defined as compounds of formula I-Qa-1 (scheme 16).

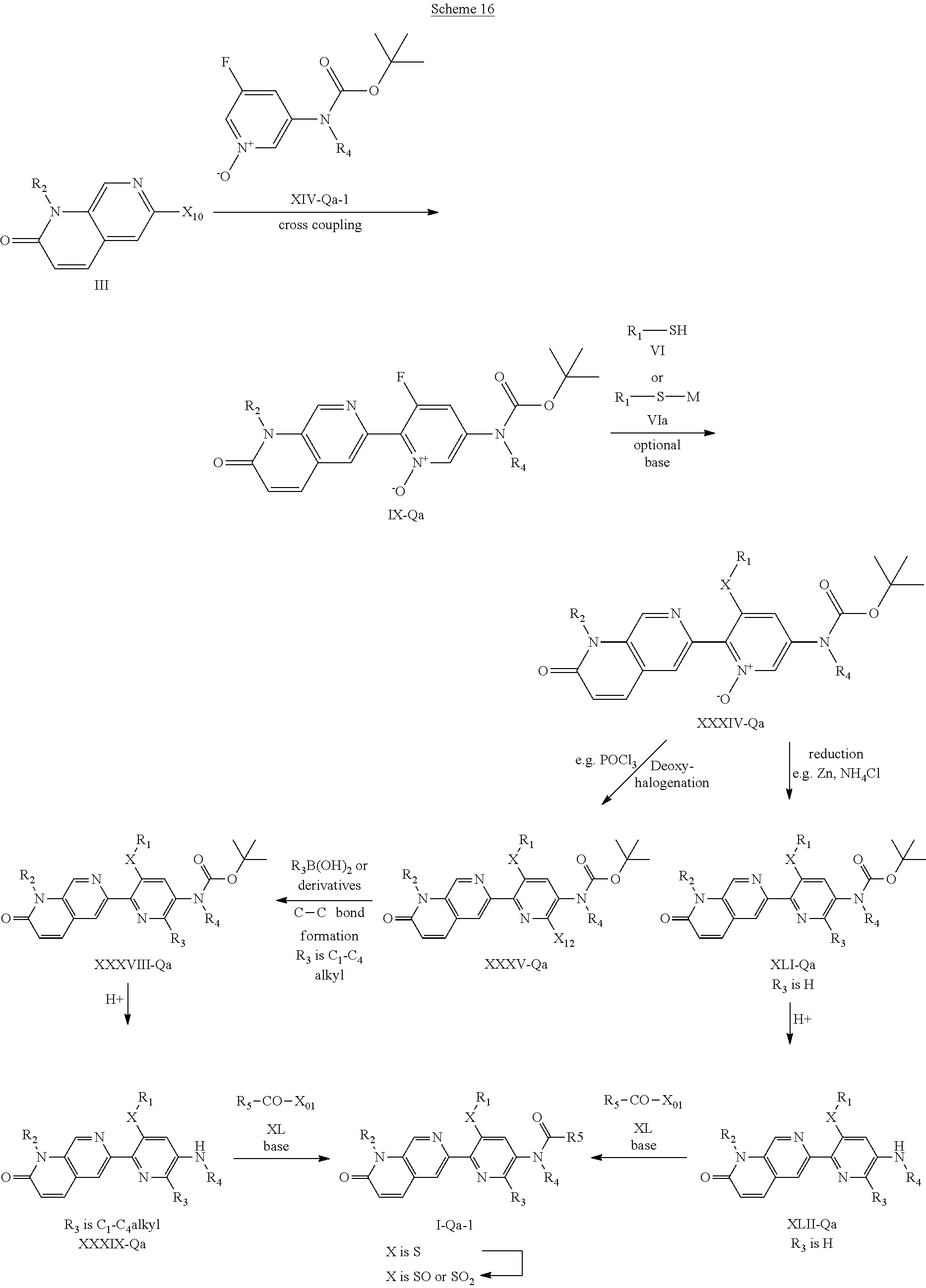

Oxidation compounds of formula I-Qa-1, wherein the substituents are as defined above, and in which X is S (sulfide), with a suitable oxidizing agent, into corresponding compounds wherein X is SO (sulfoxide) or SO₂ (sulfone) may be achieved under conditions already described above.

Compounds of formula I-Qa-1, wherein X is S and R₃ is H, and in which R₂, R₁, R₄ and R₅ are as defined in formula I, can be prepared (scheme 16) by reacting compounds of formula XLII-Qa, wherein X is S and R₃ is H, and in which R₂, R₁ and R₄ are as defined in formula I, with compounds of formula XL, wherein R₅ is as defined in formula I, and X₀₁ is a halogen, preferably chlorine (alternatively, X₀₁ is the leaving group —O(CO)R₅), in the presence of a base, such as triethylamine, N,N-diisopropyl-ethylamine or pyridine, optionally in the presence of a catalyst (such as 4-dimethylaminopyridine DMAP), in an inert solvents such as dichloromethane, tetrahydrofuran, dioxane, N,N-dimethylformamide, N,N-dimethylacetamide, acetonitrile, ethyl acetate or toluene, at temperatures between 0 and 50° C. Certain bases, such as pyridine and triethylamine, may be employed successfully as both base and solvent.

Compounds of formula XLII-Qa, wherein X is S and R₃ is H, and in which R₂, R₁ and R₄ are as defined in formula I, can be prepared from compounds of formula XLI-Qa, wherein X is S and R₃ is H, and in which R₂, R₁ and R₄ are as defined in formula I, by treatment with organic acids, for example trifluoroacetic acid, acetic acid and the like, or mineral acids such as hydrochloric acid, in inert solvents, such as dichloromethane or tetrahydrofuran THF, optionally in the presence of water, at temperatures between 0 and 80° C., by methods well known to those skilled in the art.

Alternatively, compounds of formula I-Qa-1, wherein X is SO or SO₂, may be prepared from compounds of formula XLI-Qa, wherein X is S and R₃ is H, and in which R₂, R₁ and R₄ are as defined in formula I, by involving the same chemistry as described above, but by changing the order of the steps (i.e. by running an oxidation step on XLI-Qa, wherein X is S, to form XLI-Qa, wherein X is SO or SO₂, followed by the sequence XLI-Qa (X is SO or SO₂) to XLII-Qa (X is SO or SO₂) via treatment with acids, and XLII-Qa (X is SO or SO₂) to I-Qa-1 (X is SO or SO₂) by treatment with reagents of formula XL).

Compounds of formula XLI-Qa, wherein X is S and R₃ is H, and in which R₂, R₁ and R₄ are as defined in formula I, can be prepared by deoxygenation/reduction of compounds of formula XXXIV-Qa, wherein X is S, and in which R₂, R₁ and R₄ are as defined in formula I, under conditions already described above (see scheme 5, transformation of compounds X into I-Qa).

Alternatively, compounds of formula I-Qa-1, wherein X is S and R₃ is C₁-C₄alkyl, and in which R₂, R₁, R₄ and R₅ are as defined in formula I, may be prepared (scheme 16) by reacting compounds of formula XXXIX-Qa, wherein X is S and R₃ is C₁-C₄alkyl, and in which R₂, R₁ and R₄ are as defined in formula I, with compounds of formula XL, wherein R₅ is as defined in formula I, and X₀₁ is a halogen, preferably chlorine (alternatively, X₀₁ is the leaving group —O(CO)R₅), under conditions already described above (see scheme 16, transformation of compounds XLII-Qa into I-Qa-1).

Compounds of formula XXXIX-Qa, wherein X is S and R₃ is C₁-C₄alkyl, and in which R₂, R₁ and R₄ are as defined in formula I, may be prepared by treating compounds of formula XXXVIII-Qa, wherein X is S and R₃ is C₁-C₄alkyl, and in which R₂, R₁ and R₄ are as defined in formula I, with acids under conditions already described above (see scheme 16, transformation of compounds XLI-Qa into XLII-Qa).

Compounds of formula XXXVIII-Qa, wherein X is S and R₃ is C₁-C₄alkyl, and in which R₂, R₁ and R₄ are as defined in formula I, may be prepared by reacting compounds of formula XXXV-Qa, wherein X is S, and in which R₂, R₁ and R₄ are as defined in formula I, and wherein X₁₂ is a leaving group like, for example, chlorine, bromine or iodine (preferably chlorine or bromine), or an aryl- or alkylsulfonate such as trifluoromethanesulfonate, with C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, under conditions already described above (see scheme 14, transformation of compounds XXXVa into I-Qa by means of a C—C bond formation reaction).

Compounds of formula XXXV-Qa, wherein X is S, and in which R₂, R₁ and R₄ are as defined in formula I, and wherein X₁₂ is a leaving group like, for example, chlorine, bromine or iodine (preferably chlorine or bromine), or an aryl- or alkylsulfonate such as trifluoromethanesulfonate, may be prepared from compounds of formula XXXIV-Qa, wherein X is S, and in which R₂, R₁ and R₄ are as defined in formula I, by means of a deoxyhalogenation under conditions already described above (see scheme 14, transformation of compounds XXXIVa into XXXVa).

Compounds of formula XXXIV-Qa, wherein X is S, and in which R₂, R₁ and R₄ are as defined in formula I, can be prepared by reacting compounds of formula IX-Qa, wherein R₂ and R₄ are as defined in formula I, with reagents of formula VI or VIa, wherein R₁ is as defined in formula I, under conditions already described above (see text schemes 3 and 5).

Compounds of formula IX-Qa, wherein R₂ and R₄ are as defined in formula I, can be prepared by cross-coupling compounds of formula III, wherein R₂ is as defined in formula I, and in which X₁₀ is a halogen (or a pseudo-halogen leaving group, such as a triflate), preferably bromine or chlorine, with compounds of formula XIV-Qa-1, wherein R₄ is as defined in formula I, under conditions already described above (see text schemes 7 and 15).

Compounds of formula XIV-Qa-1, wherein R₄ is as defined in formula I, are either known, commercially available, or may be prepared by methods known to a person skilled in the art or by analogy to descriptions found for example in WO 20/182577.

Oxidation of any compounds of formula XXXIV-Qa, XXXV-Qa, XXXVIII-Qa, XXXIX-Qa, XLI-Qa, XLII-Qa and I-Qa-1 in scheme 16, wherein the substituents are as defined above, and in which X is S (sulfide), with a suitable oxidizing agent, into corresponding compounds wherein X is SO (sulfoxide) or SO₂ (sulfone) may be achieved under conditions already described above.

Compounds of formula XL, wherein R₅ is as defined in formula I, and X₀₁ is a halogen, preferably chlorine (alternatively, X₀₁ is the leaving group —O(CO)R₅) are either known, commercially available or may be prepared by methods known to a person skilled in the art.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reactions are advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention, and by post modification of compounds of with reactions such as oxidation, alkylation, reduction, acylation and other methods known by those skilled in the art.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the following Tables A-1 to A-60 and Tables B-1 to B-60 below can be prepared according to the methods described above. The examples which follow are intended to illustrate the invention and show preferred compounds of formula I.

The tables A-1 to A-60 below illustrate further specific compounds of the invention.

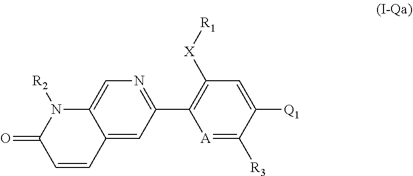

TABLE Y
Substituent definitions of Q₁

| Index | Q₁ |
|---|---|
| 1 | H |
| 2 | —N(CH₃)COCH₂CH₃ |
| 3 | Cl |
| 4 | Br |
| 5 | —N(CH₃)COcycloC3 |
| 6 | CF₃ |
| 7 | —NH₂ |
| 8 | —NH(CH₃) |
| 9 | —N(CH₃)₂ |
| 10 | —NHCOCH₃ |
| 11 | —N(CH₃)COCH₃ |
| 12 | 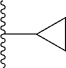 [SMILES: *C1CC1] |
| 13 | 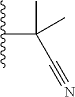 [SMILES: *C(C)(C)C#N] |
| 14 | 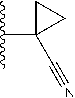 [SMILES: *C1(C#N)CC1] |
| 15 | —OCH₂CF₃ |
| 16 | 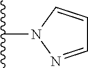 [SMILES: *n1cccn1] |
| 17 | 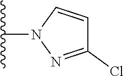 [SMILES: *n1ccc(Cl)n1] |
| 18 | 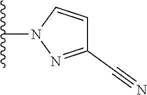 [SMILES: *n1ccc(C#N)n1] |
| 19 | 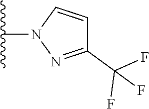 [SMILES: *n1ccc(C(F)(F)F)n1] |
| 20 | 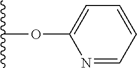 [SMILES: *Oc1ccccn1] |

In the table Y and in tables A, “cycloC3” represents cyclopropyl.

Table A-1 provides 20 compounds A-1.001 to A-1.020 of formula I-Qa wherein R₂ is CH₂CF₂CF₃, A is N, R₃ is H, X is S, R, is ethyl and Q, is as defined in table Y.

Table A-2 provides 20 compounds A-2.001 to A-2.020 of formula I-Qa wherein R₂ is CH₂CF₂CF₃, A is N, R₃ is H, X is SO, R, is ethyl and Q, is as defined in table Y.

Table A-3 provides 20 compounds A-3.001 to A-3.020 of formula I-Qa wherein R₂ is CH₂CF₂CF₃, A is N, R₃ is H, X is SO₂, R, is ethyl and Q, is as defined in table Y.

Table A-4 provides 20 compounds A-4.001 to A-4.020 of formula I-Qa wherein R₂ is CH₂CF₂CF₃, A is N, R₃ is Me, X is S, R, is ethyl and Q, is as defined in table Y.

Table A-5 provides 20 compounds A-5.001 to A-5.020 of formula I-Qa wherein R₂ is CH₂CF₂CF₃, A is N, R₃ is Me, X is SO, R, is ethyl and Q, is as defined in table Y.

Table A-6 provides 20 compounds A-6.001 to A-6.020 of formula I-Qa wherein R₂ is CH₂CF₂CF₃, A is N, R₃ is Me, X is S02, R, is ethyl and Q, is as defined in table Y.

Table A-7 provides 20 compounds A-7.001 to A-7.020 of formula I-Qa wherein R₂ is CH₂CF₂CF₃, A is CH, R₃ is H, X is 5, R, is ethyl and Q, is as defined in table Y.

Table A-8 provides 20 compounds A-8.001 to A-8.020 of formula I-Qa wherein R₂ is CH₂CF₂CF₃, A is CH, R₃ is H, X is SO, R₁ is ethyl and Q₁ is as defined in table Y.

Table A-9 provides 20 compounds A-9.001 to A-9.020 of formula I-Qa wherein R₂ is CH₂CF₂CF₃, A is CH, R₃ is H, X is SO₂, R₁ is ethyl and Q₁ is as defined in table Y.

Table A-10 provides 20 compounds A-10.001 to A-10.020 of formula I-Qa wherein R₂ is CH₂CF₂CF₃, A is CH, R₃ is Me, X is S, R₁ is ethyl and Q₁ is as defined in table Y.

Table A-11 provides 20 compounds A-11.001 to A-11.020 of formula I-Qa wherein R₂ is CH₂CF₂CF₃, A is CH, R₃ is Me, X is SO, R₁ is ethyl and Q₁ is as defined in table Y.

Table A-12 provides 20 compounds A-12.001 to A-12.020 of formula I-Qa wherein R₂ is CH₂CF₂CF₃, A is CH, R₃ is Me, X is SO₂, R₁ is ethyl and Q₁ is as defined in table Y.

Table A-13 provides 20 compounds A-13.001 to A-13.020 of formula I-Qa wherein R₂ is CH₂CF₂CHF₂, A is N, R₃ is H, X is S, R₁ is ethyl and Q₁ is as defined in table Y.

Table A-14 provides 20 compounds A-14.001 to A-14.020 of formula I-Qa wherein R₂ is CH₂CF₂CHF₂, A is N, R₃ is H, X is SO, R₁ is ethyl and Q₁ is as defined in table Y.

Table A-15 provides 20 compounds A-15.001 to A-15.020 of formula I-Qa wherein R₂ is CH₂CF₂CHF₂, A is N, R₃ is H, X is SO₂, R₁ is ethyl and Q₁ is as defined in table Y.

Table A-16 provides 20 compounds A-16.001 to A-16.020 of formula I-Qa wherein R₂ is CH₂CF₂CHF₂, A is N, R₃ is Me, X is S, R₁ is ethyl and Q₁ is as defined in table Y.

Table A-17 provides 20 compounds A-17.001 to A-17.020 of formula I-Qa wherein R₂ is CH₂CF₂CHF₂, A is N, R₃ is Me, X is SO, R₁ is ethyl and Q₁ is as defined in table Y.

Table A-18 provides 20 compounds A-18.001 to A-18.020 of formula I-Qa wherein R₂ is CH₂CF₂CHF₂, A is N, R₃ is Me, X is SO₂, R₁ is ethyl and Q₁ is as defined in table Y.

Table A-19 provides 20 compounds A-19.001 to A-19.020 of formula I-Qa wherein R₂ is CH₂CF₂CHF₂, A is CH, R₃ is H, X is S, R₁ is ethyl and Q₁ is as defined in table Y.

Table A-20 provides 20 compounds A-20.001 to A-20.020 of formula I-Qa wherein R₂ is CH₂CF₂CHF₂, A is CH, R₃ is H, X is SO, R₁ is ethyl and Q₁ is as defined in table Y.

Table A-21 provides 20 compounds A-21.001 to A-21.020 of formula I-Qa wherein R₂ is CH₂CF₂CHF₂, A is CH, R₃ is H, X is SO₂, R₁ is ethyl and Q₁ is as defined in table Y.

Table A-22 provides 20 compounds A-22.001 to A-22.020 of formula I-Qa wherein R₂ is CH₂CF₂CHF₂, A is CH, R₃ is Me, X is S, R₁ is ethyl and Q₁ is as defined in table Y.

Table A-23 provides 20 compounds A-23.001 to A-23.020 of formula I-Qa wherein R₂ is CH₂CF₂CHF₂, A is CH, R₃ is Me, X is SO, R₁ is ethyl and Q₁ is as defined in table Y.

Table A-24 provides 20 compounds A-24.001 to A-24.020 of formula I-Qa wherein R₂ is CH₂CF₂CHF₂, A is CH, R₃ is Me, X is SO₂, R₁ is ethyl and Q₁ is as defined in table Y.

Table A-25 provides 20 compounds A-25.001 to A-25.020 of formula I-Qa wherein R₂ is CH₂CF₃, A is N, R₃ is H, X is S, R₁ is ethyl and Q₁ is as defined in table Y.

Table A-26 provides 20 compounds A-26.001 to A-26.020 of formula I-Qa wherein R₂ is CH₂CF₃, A is N, R₃ is H, X is SO, R₁ is ethyl and Q₁ is as defined in table Y.

Table A-27 provides 20 compounds A-27.001 to A-27.020 of formula I-Qa wherein R₂ is CH₂CF₃, A is N, R₃ is H, X is SO₂, R₁ is ethyl and Q₁ is as defined in table Y.

Table A-28 provides 20 compounds A-28.001 to A-28.020 of formula I-Qa wherein R₂ is CH₂CF₃, A is N, R₃ is Me, X is S, R₁ is ethyl and Q₁ is as defined in table Y.

Table A-29 provides 20 compounds A-29.001 to A-29.020 of formula I-Qa wherein R₂ is CH₂CF₃, A is N, R₃ is Me, X is SO, R₁ is ethyl and Q₁ is as defined in table Y.

Table A-30 provides 20 compounds A-30.001 to A-30.020 of formula I-Qa wherein R₂ is CH₂CF₃, A is N, R₃ is Me, X is SO₂, R₁ is ethyl and Q₁ is as defined in table Y.

Table A-31 provides 20 compounds A-31.001 to A-31.020 of formula I-Qa wherein R₂ is CH₂CF₃, A is CH, R₃ is H, X is S, R₁ is ethyl and Q₁ is as defined in table Y.

Table A-32 provides 20 compounds A-32.001 to A-32.020 of formula I-Qa wherein R₂ is CH₂CF₃, A is CH, R₃ is H, X is SO, R₁ is ethyl and Q₁ is as defined in table Y.

Table A-33 provides 20 compounds A-33.001 to A-33.020 of formula I-Qa wherein R₂ is CH₂CF₃, A is CH, R₃ is H, X is SO₂, R₁ is ethyl and Q₁ is as defined in table Y.

Table A-34 provides 20 compounds A-34.001 to A-34.020 of formula I-Qa wherein R₂ is CH₂CF₃, A is CH, R₃ is Me, X is S, R₁ is ethyl and Q₁ is as defined in table Y.

Table A-35 provides 20 compounds A-35.001 to A-35.020 of formula I-Qa wherein R₂ is CH₂CF₃, A is CH, R₃ is Me, X is SO, R₁ is ethyl and Q₁ is as defined in table Y.

Table A-36 provides 20 compounds A-36.001 to A-36.020 of formula I-Qa wherein R₂ is CH₂CF₃, A is CH, R₃ is Me, X is SO₂, R₁ is ethyl and Q₁ is as defined in table Y.

Table A-37 provides 20 compounds A-37.001 to A-37.020 of formula I-Qa wherein R₂ is CH₂CHF₂, A is N, R₃ is H, X is S, R₁ is ethyl and Q₁ is as defined in table Y.

Table A-38 provides 20 compounds A-38.001 to A-38.020 of formula I-Qa wherein R₂ is CH₂CHF₂, A is N, R₃ is H, X is SO, R₁ is ethyl and Q₁ is as defined in table Y.

Table A-39 provides 20 compounds A-39.001 to A-39.020 of formula I-Qa wherein R₂ is CH₂CHF₂, A is N, R₃ is H, X is SO₂, R₁ is ethyl and Q₁ is as defined in table Y.

Table A-40 provides 20 compounds A-40.001 to A-40.020 of formula I-Qa wherein R₂ is CH₂CHF₂, A is N, R₃ is Me, X is S, R₁ is ethyl and Q₁ is as defined in table Y.

Table A-41 provides 20 compounds A-41.001 to A-41.020 of formula I-Qa wherein R₂ is CH₂CHF₂, A is N, R₃ is Me, X is SO, R₁ is ethyl and Q₁ is as defined in table Y.

Table A-42 provides 20 compounds A-42.001 to A-42.020 of formula I-Qa wherein R₂ is CH₂CHF₂, A is N, R₃ is Me, X is SO₂, R₁ is ethyl and Q₁ is as defined in table Y.

Table A-43 provides 20 compounds A-43.001 to A-43.020 of formula I-Qa wherein R₂ is CH₂CHF₂, A is CH, R₃ is H, X is S, R₁ is ethyl and Q₁ is as defined in table Y.

Table A-44 provides 20 compounds A-44.001 to A-44.020 of formula I-Qa wherein R₂ is CH₂CHF₂, A is CH, R₃ is H, X is SO, R₁ is ethyl and Q₁ is as defined in table Y.

Table A-45 provides 20 compounds A-45.001 to A-45.020 of formula I-Qa wherein R₂ is CH₂CHF₂, A is CH, R₃ is H, X is SO₂, R₁ is ethyl and Q₁ is as defined in table Y.

Table A-46 provides 20 compounds A-46.001 to A-46.020 of formula I-Qa wherein R₂ is CH₂CHF₂, A is CH, R₃ is Me, X is S, R₁ is ethyl and Q₁ is as defined in table Y.

Table A-47 provides 20 compounds A-47.001 to A-47.020 of formula I-Qa wherein R₂ is CH₂CHF₂, A is CH, R₃ is Me, X is SO, R₁ is ethyl and Q₁ is as defined in table Y.

Table A-48 provides 20 compounds A-48.001 to A-48.020 of formula I-Qa wherein R₂ is CH₂CHF₂, A is CH, R₃ is Me, X is SO₂, R₁ is ethyl and Q₁ is as defined in table Y.

Table A-49 provides 20 compounds A-49.001 to A-49.020 of formula I-Qa wherein R₂ is CH₂CF₂CHFCF₃, A is N, R₃ is H, X is S, R₁ is ethyl and Q₁ is as defined in table Y.

Table A-50 provides 20 compounds A-50.001 to A-50.020 of formula I-Qa wherein R₂ is CH₂CF₂CHFCF₃, A is N, R₃ is H, X is SO, R₁ is ethyl and Q₁ is as defined in table Y.

Table A-51 provides 20 compounds A-51.001 to A-51.020 of formula I-Qa wherein R₂ is CH₂CF₂CHFCF₃, A is N, R₃ is H, X is SO₂, R₁ is ethyl and Q₁ is as defined in table Y.

Table A-52 provides 20 compounds A-52.001 to A-52.020 of formula I-Qa wherein R₂ is CH₂CF₂CHFCF₃, A is N, R₃ is Me, X is S, R₁ is ethyl and Q₁ is as defined in table Y.

Table A-53 provides 20 compounds A-53.001 to A-53.020 of formula I-Qa wherein R₂ is CH₂CF₂CHFCF₃, A is N, R₃ is Me, X is SO, R₁ is ethyl and Q₁ is as defined in table Y.

Table A-54 provides 20 compounds A-54.001 to A-54.020 of formula I-Qa wherein R₂ is CH₂CF₂CHFCF₃, A is N, R₃ is Me, X is SO₂, R₁ is ethyl and Q₁ is as defined in table Y.

Table A-55 provides 20 compounds A-55.001 to A-55.020 of formula I-Qa wherein R₂ is CH₂CF₂CHFCF₃, A is CH, R₃ is H, X is S, R₁ is ethyl and Q₁ is as defined in table Y.

Table A-56 provides 20 compounds A-56.001 to A-56.020 of formula I-Qa wherein R₂ is CH₂CF₂CHFCF₃, A is CH, R₃ is H, X is SO, R₁ is ethyl and Q₁ is as defined in table Y.

Table A-57 provides 20 compounds A-57.001 to A-57.020 of formula I-Qa wherein R₂ is CH₂CF₂CHFCF₃, A is CH, R₃ is H, X is SO₂, R₁ is ethyl and Q₁ is as defined in table Y.

Table A-58 provides 20 compounds A-58.001 to A-58.020 of formula I-Qa wherein R₂ is CH₂CF₂CHFCF₃, A is CH, R₃ is Me, X is S, R₁ is ethyl and Q₁ is as defined in table Y.

Table A-59 provides 20 compounds A-59.001 to A-59.020 of formula I-Qa wherein R₂ is CH₂CF₂CHFCF₃, A is CH, R₃ is Me, X is SO, R₁ is ethyl and Q₁ is as defined in table Y.

Table A-60 provides 20 compounds A-60.001 to A-60.020 of formula I-Qa wherein R₂ is CH₂CF₂CHFCF₃, A is CH, R₃ is Me, X is SO₂, R₁ is ethyl and Q₁ is as defined in table Y.

The tables B-1 to B-60 below illustrate further specific compounds of the invention.

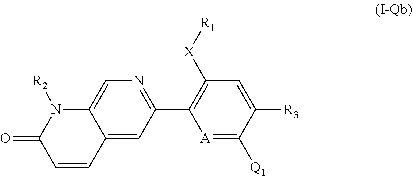

TABLE Z
Substituent definitions of Q₁

| Index | Q₁ |
|---|---|
| 1 | H |
| 2 | Cl |
| 3 | Br |
| 4 | —N(CH₃)COCH₂CH₃ |
| 5 | —NH₂ |
| 6 | —NH(CH₃) |
| 7 | —N(CH₃)₂ |
| 8 | —NHCOCH₃ |
| 9 | —N(CH₃)COCH₃ |
| 10 | —NHCOcycloC3 |
| 11 | —N(CH₃)COcycloC3 |
| 12 | 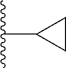 [SMILES: *C1CC1] |
| 13 | —N(H)CONH₂ |
| 14 | —N(H)CONH(CH₃) |
| 15 | —N(H)CON(CH₃)₂ |
| 16 | —N(CH₃)CONH₂ |
| 17 | —N(CH₃)CONH(CH₃) |
| 18 | —N(CH₃)CON(CH₃)₂ |
| 19 | 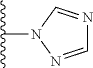 [SMILES: *n1cncn1] |
| 20 | 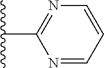 [SMILES: *c1ncccn1] |
| 21 | 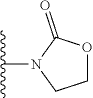 [SMILES: *N1CCOC1=O] |

In the table Z and in tables B, “cycloC3” represents cyclopropyl.

Table B-1 provides 21 compounds B-1.001 to B-1.021 of formula I-Qb wherein R₂ is CH₂CF₂CF₃, A is N, R₃ is H, X is S, R₁ is ethyl and Q₁ is as defined in table Z.

Table B-2 provides 21 compounds B-2.001 to B-2.021 of formula I-Qb wherein R₂ is CH₂CF₂CF₃, A is N, R₃ is H, X is SO, R₁ is ethyl and Q₁ is as defined in table Z.

Table B-3 provides 21 compounds B-3.001 to B-3.021 of formula I-Qb wherein R₂ is CH₂CF₂CF₃, A is N, R₃ is H, X is SO₂, R₁ is ethyl and Q₁ is as defined in table Z.

Table B-4 provides 21 compounds B-4.001 to B-4.021 of formula I-Qb wherein R₂ is CH₂CF₂CF₃, A is N, R₃ is Me, X is S, R₁ is ethyl and Q₁ is as defined in table Z.

Table B-5 provides 21 compounds B-5.001 to B-5.021 of formula I-Qb wherein R₂ is CH₂CF₂CF₃, A is N, R₃ is Me, X is SO, R₁ is ethyl and Q₁ is as defined in table Z.

Table B-6 provides 21 compounds B-6.001 to B-6.021 of formula I-Qb wherein R₂ is CH₂CF₂CF₃, A is N, R₃ is Me, X is SO₂, R₁ is ethyl and Q₁ is as defined in table Z.

Table B-7 provides 21 compounds B-7.001 to B-7.021 of formula I-Qb wherein R₂ is CH₂CF₂CF₃, A is CH, R₃ is H, X is S, R₁ is ethyl and Q₁ is as defined in table Z.

Table B-8 provides 21 compounds B-8.001 to B-8.021 of formula I-Qb wherein R₂ is CH₂CF₂CF₃, A is CH, R₃ is H, X is SO, R₁ is ethyl and Q₁ is as defined in table Z.

Table B-9 provides 21 compounds B-9.001 to B-9.021 of formula I-Qb wherein R₂ is CH₂CF₂CF₃, A is CH, R₃ is H, X is SO₂, R₁ is ethyl and Q₁ is as defined in table Z.

Table B-10 provides 21 compounds B-10.001 to B-10.021 of formula I-Qb wherein R₂ is CH₂CF₂CF₃, A is CH, R₃ is Me, X is S, R₁ is ethyl and Q₁ is as defined in table Z.

Table B-11 provides 21 compounds B-11.001 to B-11.021 of formula I-Qb wherein R₂ is CH₂CF₂CF₃, A is CH, R₃ is Me, X is SO, R₁ is ethyl and Q₁ is as defined in table Z.

Table B-12 provides 21 compounds B-12.001 to B-12.021 of formula I-Qb wherein R₂ is CH₂CF₂CF₃, A is CH, R₃ is Me, X is SO₂, R₁ is ethyl and Q₁ is as defined in table Z.

Table B-13 provides 21 compounds B-13.001 to B-13.021 of formula I-Qb wherein R₂ is CH₂CF₂CHF₂, A is N, R₃ is H, X is S, R₁ is ethyl and Q₁ is as defined in table Z.

Table B-14 provides 21 compounds B-14.001 to B-14.021 of formula I-Qb wherein R₂ is CH₂CF₂CHF₂, A is N, R₃ is H, X is SO, R₁ is ethyl and Q₁ is as defined in table Z.

Table B-15 provides 21 compounds B-15.001 to B-15.021 of formula I-Qb wherein R₂ is CH₂CF₂CHF₂, A is N, R₃ is H, X is SO₂, R₁ is ethyl and Q₁ is as defined in table Z.

Table B-16 provides 21 compounds B-16.001 to B-16.021 of formula I-Qb wherein R₂ is CH₂CF₂CHF₂, A is N, R₃ is Me, X is S, R₁ is ethyl and Q₁ is as defined in table Z.

Table B-17 provides 21 compounds B-17.001 to B-17.021 of formula I-Qb wherein R₂ is CH₂CF₂CHF₂, A is N, R₃ is Me, X is SO, R₁ is ethyl and Q₁ is as defined in table Z.

Table B-18 provides 21 compounds B-18.001 to B-18.021 of formula I-Qb wherein R₂ is CH₂CF₂CHF₂, A is N, R₃ is Me, X is SO₂, R₁ is ethyl and Q₁ is as defined in table Z.

Table B-19 provides 21 compounds B-19.001 to B-19.021 of formula I-Qb wherein R₂ is CH₂CF₂CHF₂, A is CH, R₃ is H, X is S, R₁ is ethyl and Q₁ is as defined in table Z.

Table B-20 provides 21 compounds B-20.001 to B-20.021 of formula I-Qb wherein R₂ is CH₂CF₂CHF₂, A is CH, R₃ is H, X is SO, R₁ is ethyl and Q₁ is as defined in table Z.

Table B-21 provides 21 compounds B-21.001 to B-21.021 of formula I-Qb wherein R₂ is CH₂CF₂CHF₂, A is CH, R₃ is H, X is SO₂, R₁ is ethyl and Q₁ is as defined in table Z.

Table B-22 provides 21 compounds B-22.001 to B-22.021 of formula I-Qb wherein R₂ is CH₂CF₂CHF₂, A is CH, R₃ is Me, X is S, R₁ is ethyl and Q₁ is as defined in table Z.

Table B-23 provides 21 compounds B-23.001 to B-23.021 of formula I-Qb wherein R₂ is CH₂CF₂CHF₂, A is CH, R₃ is Me, X is SO, R₁ is ethyl and Q₁ is as defined in table Z.

Table B-24 provides 21 compounds B-24.001 to B-24.021 of formula I-Qb wherein R₂ is CH₂CF₂CHF₂, A is CH, R₃ is Me, X is SO₂, R₁ is ethyl and Q₁ is as defined in table Z.

Table B-25 provides 21 compounds B-25.001 to B-25.021 of formula I-Qb wherein R₂ is CH₂CF₃, A is N, R₃ is H, X is S, R₁ is ethyl and Q₁ is as defined in table Z.

Table B-26 provides 21 compounds B-26.001 to B-26.021 of formula I-Qb wherein R₂ is CH₂CF₃, A is N, R₃ is H, X is SO, R₁ is ethyl and Q₁ is as defined in table Z.

Table B-27 provides 21 compounds B-27.001 to B-27.021 of formula I-Qb wherein R₂ is CH₂CF₃, A is N, R₃ is H, X is SO₂, R₁ is ethyl and Q₁ is as defined in table Z.

Table B-28 provides 21 compounds B-28.001 to B-28.021 of formula I-Qb wherein R₂ is CH₂CF₃, A is N, R₃ is Me, X is S, R₁ is ethyl and Q₁ is as defined in table Z.

Table B-29 provides 21 compounds B-29.001 to B-29.021 of formula I-Qb wherein R₂ is CH₂CF₃, A is N, R₃ is Me, X is SO, R₁ is ethyl and Q₁ is as defined in table Z.

Table B-30 provides 21 compounds B-30.001 to B-30.021 of formula I-Qb wherein R₂ is CH₂CF₃, A is N, R₃ is Me, X is SO₂, R₁ is ethyl and Q₁ is as defined in table Z.

Table B-31 provides 21 compounds B-31.001 to B-31.021 of formula I-Qb wherein R₂ is CH₂CF₃, A is CH, R₃ is H, X is S, R₁ is ethyl and Q₁ is as defined in table Z.

Table B-32 provides 21 compounds B-32.001 to B-32.021 of formula I-Qb wherein R₂ is CH₂CF₃, A is CH, R₃ is H, X is SO, R₁ is ethyl and Q₁ is as defined in table Z.

Table B-33 provides 21 compounds B-33.001 to B-33.021 of formula I-Qb wherein R₂ is CH₂CF₃, A is CH, R₃ is H, X is SO₂, R₁ is ethyl and Q₁ is as defined in table Z.

Table B-34 provides 21 compounds B-34.001 to B-34.021 of formula I-Qb wherein R₂ is CH₂CF₃, A is CH, R₃ is Me, X is S, R₁ is ethyl and Q₁ is as defined in table Z.

Table B-35 provides 21 compounds B-35.001 to B-35.021 of formula I-Qb wherein R₂ is CH₂CF₃, A is CH, R₃ is Me, X is SO, R₁ is ethyl and Q₁ is as defined in table Z.

Table B-36 provides 21 compounds B-36.001 to B-36.021 of formula I-Qb wherein R₂ is CH₂CF₃, A is CH, R₃ is Me, X is SO₂, R₁ is ethyl and Q₁ is as defined in table Z.

Table B-37 provides 21 compounds B-37.001 to B-37.021 of formula I-Qb wherein R₂ is CH₂CHF₂, A is N, R₃ is H, X is S, R₁ is ethyl and Q₁ is as defined in table Z.

Table B-38 provides 21 compounds B-38.001 to B-38.021 of formula I-Qb wherein R₂ is CH₂CHF₂, A is N, R₃ is H, X is SO, R₁ is ethyl and Q₁ is as defined in table Z.

Table B-39 provides 21 compounds B-39.001 to B-39.021 of formula I-Qb wherein R₂ is CH₂CHF₂, A is N, R₃ is H, X is SO₂, R₁ is ethyl and Q₁ is as defined in table Z.

Table B-40 provides 21 compounds B-40.001 to B-40.021 of formula I-Qb wherein R₂ is CH₂CHF₂, A is N, R₃ is Me, X is S, R₁ is ethyl and Q₁ is as defined in table Z.

Table B-41 provides 21 compounds B-41.001 to B-41.021 of formula I-Qb wherein R₂ is CH₂CHF₂, A is N, R₃ is Me, X is SO, R₁ is ethyl and Q₁ is as defined in table Z.

Table B-42 provides 21 compounds B-42.001 to B-42.021 of formula I-Qb wherein R₂ is CH₂CHF₂, A is N, R₃ is Me, X is SO₂, R₁ is ethyl and Q₁ is as defined in table Z.

Table B-43 provides 21 compounds B-43.001 to B-43.021 of formula I-Qb wherein R₂ is CH₂CHF₂, A is CH, R₃ is H, X is S, R₁ is ethyl and Q₁ is as defined in table Z.

Table B-44 provides 21 compounds B-44.001 to B-44.021 of formula I-Qb wherein R₂ is CH₂CHF₂, A is CH, R₃ is H, X is SO, R₁ is ethyl and Q₁ is as defined in table Z.

Table B-45 provides 21 compounds B-45.001 to B-45.021 of formula I-Qb wherein R₂ is CH₂CHF₂, A is CH, R₃ is H, X is SO₂, R₁ is ethyl and Q₁ is as defined in table Z.

Table B-46 provides 21 compounds B-46.001 to B-46.021 of formula I-Qb wherein R₂ is CH₂CHF₂, A is CH, R₃ is Me, X is S, R₁ is ethyl and Q₁ is as defined in table Z.

Table B-47 provides 21 compounds B-47.001 to B-47.021 of formula I-Qb wherein R₂ is CH₂CHF₂, A is CH, R₃ is Me, X is SO, R₁ is ethyl and Q₁ is as defined in table Z.

Table B-48 provides 21 compounds B-48.001 to B-48.021 of formula I-Qb wherein R₂ is CH₂CHF₂, A is CH, R₃ is Me, X is SO₂, R₁ is ethyl and Q₁ is as defined in table Z.

Table B-49 provides 21 compounds B-49.001 to B-49.021 of formula I-Qb wherein R₂ is CH₂CF₂CHFCF₃, A is N, R₃ is H, X is S, R₁ is ethyl and Q₁ is as defined in table Z.

Table B-50 provides 21 compounds B-50.001 to B-50.021 of formula I-Qb wherein R₂ is CH₂CF₂CHFCF₃, A is N, R₃ is H, X is SO, R₁ is ethyl and Q₁ is as defined in table Z.

Table B-51 provides 21 compounds B-51.001 to B-51.021 of formula I-Qb wherein R₂ is CH₂CF₂CHFCF₃, A is N, R₃ is H, X is SO₂, R₁ is ethyl and Q₁ is as defined in table Z.

Table B-52 provides 21 compounds B-52.001 to B-52.021 of formula I-Qb wherein R₂ is CH₂CF₂CHFCF₃, A is N, R₃ is Me, X is S, R₁ is ethyl and Q₁ is as defined in table Z.

Table B-53 provides 21 compounds B-53.001 to B-53.021 of formula I-Qb wherein R₂ is CH₂CF₂CHFCF₃, A is N, R₃ is Me, X is SO, R₁ is ethyl and Q₁ is as defined in table Z.

Table B-54 provides 21 compounds B-54.001 to B-54.021 of formula I-Qb wherein R₂ is CH₂CF₂CHFCF₃, A is N, R₃ is Me, X is SO₂, R₁ is ethyl and Q₁ is as defined in table Z.

Table B-55 provides 21 compounds B-55.001 to B-55.021 of formula I-Qb wherein R₂ is CH₂CF₂CHFCF₃, A is CH, R₃ is H, X is S, R₁ is ethyl and Q₁ is as defined in table Z.

Table B-56 provides 21 compounds B-56.001 to B-56.021 of formula I-Qb wherein R₂ is CH₂CF₂CHFCF₃, A is CH, R₃ is H, X is SO, R₁ is ethyl and Q₁ is as defined in table Z.

Table B-57 provides 21 compounds B-57.001 to B-57.021 of formula I-Qb wherein R₂ is CH₂CF₂CHFCF₃, A is CH, R₃ is H, X is SO₂, R₁ is ethyl and Q₁ is as defined in table Z.

Table B-58 provides 21 compounds B-58.001 to B-58.021 of formula I-Qb wherein R₂ is CH₂CF₂CHFCF₃, A is CH, R₃ is Me, X is S, R₁ is ethyl and Q₁ is as defined in table Z.

Table B-59 provides 21 compounds B-59.001 to B-59.021 of formula I-Qb wherein R₂ is CH₂CF₂CHFCF₃, A is CH, R₃ is Me, X is SO, R₁ is ethyl and Q₁ is as defined in table Z.

Table B-60 provides 21 compounds B-60.001 to B-60.021 of formula I-Qb wherein R₂ is CH₂CF₂CHFCF₃, A is CH, R₃ is Me, X is SO₂, R₁ is ethyl and Q₁ is as defined in table Z.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i.e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

Examples of the above-mentioned animal pests are:

from the order Acarina, for example,

Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;

from the order Anoplura, for example,

Haematopinus spp., Linognathus spp., Pediculus spp., Pemphigus spp. and Phylloxera spp.; from the order Coleoptera, for example,

Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.; from the order Diptera, for example,

Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;

from the order Hemiptera, for example,

Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euschistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens;

Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;

from the order Hymenoptera, for example,

Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;

from the order Isoptera, for example,

Coptotermes spp, Corniternes cumulans, Incisitermes spp, Macrotermes spp, Mastotermes spp, Microtermes spp, Reticulitermes spp.; Solenopsis geminate from the order Lepidoptera, for example,

Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Gra-pholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypi-ela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.; from the order Mallophaga, for example,

Damalinea spp. and Trichodectes spp.;

from the order Orthoptera, for example,

Blatta spp., Blattella spp., Gryllotalpa spp., Leucophaea maderae, Locusta spp., Neocurtilla hexadactyla, Periplaneta spp., Scapteriscus spp, and Schistocerca spp.;

from the order Psocoptera, for example,

Liposcelis spp.;

from the order Siphonaptera, for example,

Ceratophyllus spp., Ctenocephalides spp. and Xenopsylla cheopis;

from the order Thysanoptera, for example,

Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;

from the order Thysanura, for example, Lepisma saccharina.

The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrullus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca(preferably in vegetables, vineyards), Leptinotarsa (preferably in potatoes) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca(preferably in vegetables, vineyards), Leptinotarsa (preferably in potatoes) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1 Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example,

ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| Family | Species | Host or Crop Infested |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| Family | Species | Host or Crop Infested |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| Family | Species | Host or Crop Infested |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirexjuvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances. A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers. The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

Emulsifiable concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

solid carrier: 99.9 to 90%, preferably 99.9 to 99%

Suspension concentrates:

active ingredient: 5 to 75%, preferably 10 to 50%

water: 94 to 24%, preferably 88 to 30%

surface-active agent: 1 to 40%, preferably 2 to 30%

Wettable powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension Concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil | 1% |
| (in the form of a 75 % emulsion in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil | 0.2% |
| (in the form of a 75 % emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### Preparatory Examples

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻. LCMS Methods:

Method 1:

Spectra were recorded on a Mass Spectrometer from Waters (SQD Single quadrupole mass spectrometer) equipped with an electrospray source (Polarity: positive or negative ions, Full Scan, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and a H-Class UPLC from Waters: Binary pump, heated column compartment and diode-array detector. Column: Waters UPLC HSS T3 C18, 1.8 μm, 30×2.1 mm, Temp: 40° C., DAD Wavelength range (nm): 210 to 400, Solvent Gradient: A=water+5% Acetonitrile+0.1% HCOOH, B=Acetonitrile+0.05% HCOOH: gradient: 0 min 10% B; 0.-0.2 min 10-50% B; 0.2-0.7 min 50-100% B; 0.7-1.3 min 100% B; 1.3-1.4 min 100-10% B; 1.4-1.6 min 10% B; Flow (mL/min) 0.6.

Method 2:

Spectra were recorded on a Mass Spectrometer from Agilent Technologies (6410 Triple Quadrupole mass spectrometer) equipped with an equipped with an electrospray source (Polarity: positive or negative ions, MS2 Scan, Capillary: 4.00 kV, Fragmentor: 100 V, Desolvatation Temperature: 350° C., Gas Flow: 11 L/min, Nebulizer Gas: 45 psi, Mass range: 110 to 1000 Da) and a 1200 Series HPLC from Agilent: quaternary pump, heated column compartment and diode-array detector. Column: KINETEX EVO C18, 2.6 m, 50×4.6 mm, Temp: 40° C., DAD Wavelength range (nm): 210 to 400,

Solvent Gradient: A=water+5% Acetonitrile+0.1% HCOOH, B=Acetonitrile+0.1% HCOOH: gradient: 0 min 0% B, 100% A; 0.9-1.8 min 100% B; Flow (mL/min) 1.8.

### Example H1: Preparation of 6-(3-ethylsulfonyl-2-pyridyl)-1-(2,2,3,3,3-pentafluoropropyl)-1,7-naphthyridin-2-one (compound P2)

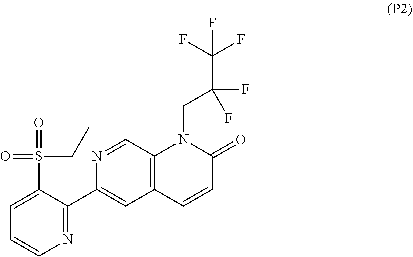 [SMILES: CCS(=O)(=O)c1cccnc1-c1cc2ccc(=O)n(CC(F)(F)C(F)(F)F)c2cn1]

### Step 1: Preparation of ethyl 3-(2-chloro-5-nitro-4-pyridyl)-2-oxo-propanoate (intermediate I-1)

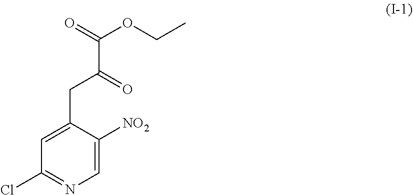 [SMILES: CCOC(=O)C(=O)Cc1cc(Cl)ncc1[N+](=O)[O-]]

To a solution of 2-chloro-4-methyl-5-nitropyridine (CAS 23056-33-9, 1.00 g, 5.79 mmol) in diethyl oxalate (7.59 mL, 54.5 mmol) under nitrogen atmosphere was added 1,8-diazabicyclo[5.4.0]undec-7-ene (1.15 mL, 7.53 mmol) at room temperature over a period of 10 minutes. The reaction mixture was stirred at room temperature overnight. The reaction mixture was then poured into ice cold water (10 mL), acidified with 2N hydrochloric acid (3 mL) and stirred for 5 minutes. The solvent was decanted off and the resulting residue was stirred in ice cold methanol for 20 minutes. The precipitate was filtered and dried under reduced pressure to afford the desired product (1.42 g). LCMS (method 1): 273 (M+H)⁺, Rt 0.99 min.

### Step 2: Preparation of ethyl 3-(2-chloro-5-nitro-4-pyridyl)-2-hydroxy-propanoate (intermediate I-2)

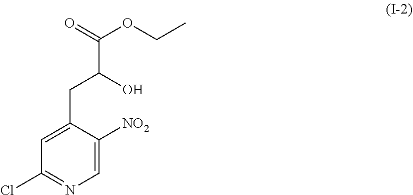 [SMILES: CCOC(=O)C(O)Cc1cc(Cl)ncc1[N+](=O)[O-]]

To an ice cooled solution of ethyl 3-(2-chloro-5-nitro-4-pyridyl)-2-oxo-propanoate (intermediate I-1 prepared as described above, 0.500 g, 1.80 mmol) in tetrahydrofuran (4 mL) and water (1 mL) was added portionwise at 0° C. sodium borohydride (70 mg, 1.8 mmol). The reaction mixture was stirred for 15 minutes at 0° C. Ice cold water was added to the reaction mixture and it was quenched at 0° C. with ammonium chloride sat. aq. The resulting suspension was extracted three times with ethyl acetate. The combined organic layers were dried over sodium sulfate, filtered and concentrated under reduced pressure to afford the desired product (0.480 g) which was used without further purification. LCMS (method 1): 275 (M+H)⁺, Rt 0.89 min.

### Step 3: Preparation of 6-chloro-1H-1,7-naphthyridin-2-one (intermediate I-3)

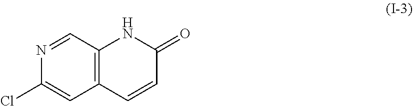 [SMILES: O=c1ccc2cc(Cl)ncc2[nH]1]

To a solution of ethyl 3-(2-chloro-5-nitro-4-pyridyl)-2-hydroxy-propanoate (intermediate I-2 prepared as described above, 0.400 g, 0.970 mmol) in acetic acid (6 mL) was added iron (0.220 g, 3.90 mmol) at room temperature. The reaction mixture was heated up to 70° C. for 30 minutes. To this reaction mixture was then added at 70° C. 1,4-dioxane (4 mL) and 6N hydrochloric acid (3 mL). The temperature was increased to 90° C. and kept stirring for 4 hours. After cooling down to room temperature, the reaction mixture was filtered over celite and washed with ethyl acetate. The filtrate was concentrated under reduced pressure. The obtained residue was diluted with water (20 mL), cooled to 0° C. and neutralized with sodium bicarbonate sat. aq. (25 mL). The suspension was filtered and dried under reduced pressure. The crude residue was taken in ethanol:1,2-dichloroethane (1:1, 20 mL) and heated to 70° C. for 30 minutes. The resulting hot solution was filtered over celite and concentrated under reduced pressure to afford the desired product as a brown solid (0.125 g). LCMS (method 1): 181 (M+H)⁺, Rt 0.38 min. ¹H NMR (400 MHz, DMSO-d₆) δ ppm 6.82 (d, J=9.54 Hz, 1H) 7.83 (s, 1H) 7.92 (d, J=9.54 Hz, 1H) 8.44 (s, 1H) 11.99-12.23 (m, 1H).

### Step 4: Preparation of 6-chloro-1-(2,2,3,3,3-pentafluoropropyl)-1,7-naphthyridin-2-one (intermediate I-4)

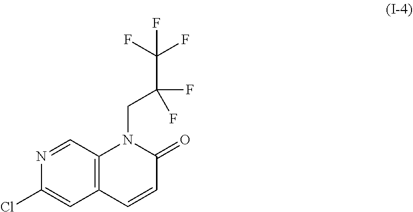 [SMILES: O=c1ccc2cc(Cl)ncc2n1CC(F)(F)C(F)(F)F]

To a solution of 6-chloro-1H-1,7-naphthyridin-2-one (intermediate I-3 prepared as described above, 2.50 g, 13.8 mmol) in tetrahydrofuran (50 mL) were added at room temperature potassium carbonate (4.98 g, 36.0 mmol) and 2,2,3,3,3-pentafluoropropyl trifluoromethanesulfonate (3.31 mL, 19.4 mmol). The reaction mixture was stirred at 70° C. for 9 hours and then at 50° C. overnight. After cooling down to room temperature, it was poured into water and extracted with ethyl acetate. The combined organic layers were washed with brine, dried over sodium sulfate, filtered and concentrated under reduced pressure. Purification of the crude material by flash chromatography over silica gel (ethyl acetate in cyclohexane) afforded the desired product (0.900 g). LCMS (method 1): 313 (M+H)⁺, Rt 1.03 min.

¹H NMR (400 MHz, CDCl₃) δ ppm 5.03 (br s, 2H) 6.97 (d, J=9.66 Hz, 1H) 7.51 (s, 1H) 7.67 (d, J=9.66 Hz, 1H) 8.57 (s, 1H).

### Step 5: Preparation of 6-(3-fluoro-2-pyridyl)-1-(2,2,3,3,3-pentafluoropropyl)-1,7-naphthyridin-2-one (intermediate I-5)

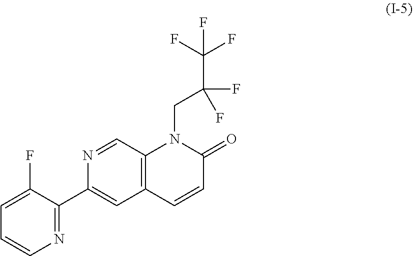 [SMILES: O=c1ccc2cc(-c3ncccc3F)ncc2n1CC(F)(F)C(F)(F)F]

To a solution of 6-chloro-1-(2,2,3,3,3-pentafluoropropyl)-1,7-naphthyridin-2-one (intermediate I-4 prepared as described above, 0.800 g, 2.56 mmol) in 1,4-dioxane (15 mL) was added 2-dicyclohexylphosphino-2′,4′,6′-triisopropylbiphenyl (XPhos) (0.30 g, 0.61 mmol). The reaction mixture was degassed under nitrogen atmosphere for 15 minutes, before adding palladium(II) acetate (59 mg, 0.26 mmol). The reaction mixture was again degassed for additional 15 minutes, before adding tributyl-(3-fluoro-2-pyridyl)stannane (1.51 g, 3.84 mmol). It was then heated up to 90° C. and stirred overnight. After cooling down to room temperature, it was poured into water and extracted with ethyl acetate. The combined organic layers were washed with brine, dried over sodium sulfate, filtered and concentrated under reduced pressure. Purification of the crude material by flash chromatography over silica gel (ethyl acetate in cyclohexane) afforded the desired product (0.210 g). LCMS (method 1): 374 (M+H)⁺, Rt 0.97 min. ¹H NMR (400 MHz, CDCl₃) δ ppm 4.92-5.32 (m, 2H) 6.99 (d, J=9.66 Hz, 1H) 7.40-7.49 (m, 1H) 7.53-7.68 (m, 1H) 7.77-7.88 (d, J=9.66 Hz, 1H) 8.27 (s, 1H) 8.60-8.68 (m, 1H) 9.01 (br s, 1H).

### Step 6: Preparation of 6-(3-ethylsulfanyl-2-pyridyl)-1-(2,2,3,3,3-pentafluoropropyl)-1,7-naphthyridin-2-one (compound P1)

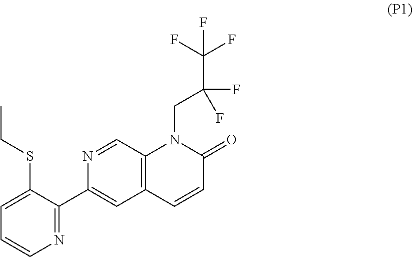 [SMILES: CCSc1cccnc1-c1cc2ccc(=O)n(CC(F)(F)C(F)(F)F)c2cn1]

To a solution of 6-(3-fluoro-2-pyridyl)-1-(2,2,3,3,3-pentafluoropropyl)-1,7-naphthyridin-2-one (intermediate I-5 prepared as described above, 0.200 g, 0.536 mmol) in dry N,N-dimethylformamide (5 mL) was added at room temperature under nitrogen atmosphere ethylsulfanylsodium (0.110 g, 1.18 mmol). The reaction mixture was stirred at room temperature for 90 minutes. Water was then added and it was extracted with ethyl acetate. The combined organic layers were washed with brine, dried over sodium sulfate, filtered and concentrated under reduced pressure. Purification of the crude material by flash chromatography over silica gel (gradient ethyl acetate in cyclohexane) afforded the desired product (55 mg). LCMS (method 1): 416 (M+H)⁺, Rt 1.07 min.

### Step 7: Preparation of 6-(3-ethylsulfonyl-2-pyridyl)-1-(2,2,3,3,3-pentafluoropropyl)-1,7-naphthyridin-2-one (title compound P2)

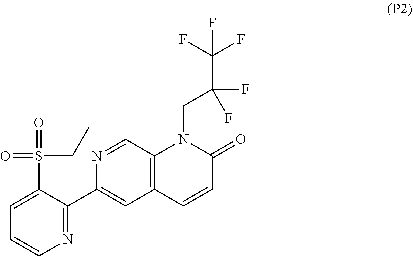 [SMILES: CCS(=O)(=O)c1cccnc1-c1cc2ccc(=O)n(CC(F)(F)C(F)(F)F)c2cn1]

To a solution of 6-(3-ethylsulfanyl-2-pyridyl)-1-(2,2,3,3,3-pentafluoropropyl)-1,7-naphthyridin-2-one (intermediate I-6 prepared as described above, 65 mg, 0.16 mmol) in dichloromethane (5 mL) was added at 0° C. 3-chloroperbenzoic acid (85 mg, 0.34 mmol). The reaction mixture was stirred at room temperature for 2 hours. It was then quenched with potassium carbonate sat. aq. and water, and extracted with ethyl acetate. The combined organic layers were washed with brine, dried over sodium sulfate, filtered and concentrated under reduced pressure. Purification of the crude material by flash chromatography over silica gel (gradient ethyl acetate in cyclohexane) afforded the desired product as an off-white solid (10 mg), mp 217-219° C. LCMS (method 1): 448 (M+H)⁺, Rt 0.97 min.

### Example H2: Preparation of 1-[5-ethylsulfonyl-6-[2-oxo-1-(2,2,3,3,3-pentafluoropropyl)-1,7-naphthyridin-6-yl]-3-pyridylicyclopropanecarbonitrile (compound P3)

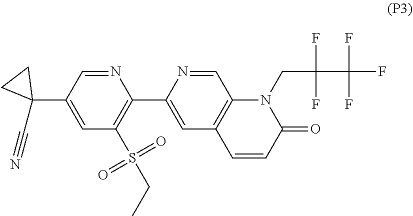 [SMILES: CCS(=O)(=O)c1cc(C2(C#N)CC2)cnc1-c1cc2ccc(=O)n(CC(F)(F)C(F)(F)F)c2cn1]

### Step 1: Preparation of ethyl (E)-3-(2-bromo-5-nitro-4-pyridyl)-2-hydroxy-prop-2-enoate (intermediate I-6)

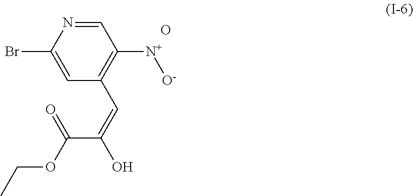

To a solution of ethyl 3-(2-bromo-5-nitro-4-pyridyl)-2-oxo-propanoate (CAS: 800401-69-8, 200 mg, 0.631 mmol) in tetrahydrofuran (4 ml) was added sodium borohydride (30 mg, 0.757 mmol) in three portions at 0° C. The mixture was warmed to 5° C. over 30 minutes before water (5 ml) was added. The reaction mixture was neutralized with aqueous saturated ammonium chloride (3 ml) and the product extracted with ethyl acetate (3×20 ml). The combined organic layers were dried over anhydrous sodium sulfate, filtered and concentrated under reduced pressure. Purification of the crude material by flash chromatography over silica gel (gradient ethyl acetate in cyclohexane) afforded the desired product as a thick yellow oil (110 mg). LCMS (method 1): 315/317 (M−H)⁻, Rt 0.91 min.

### Step 2: Preparation of (E)-3-(5-amino-2-bromo-4-pyridyl)prop-2-enoic acid (intermediate I-7)

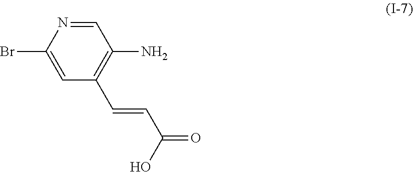 [SMILES: Nc1cnc(Br)cc1/C=C/C(=O)O]

Ethyl (E)-3-(2-bromo-5-nitro-4-pyridyl)-2-hydroxy-prop-2-enoate (intermediate I-6 prepared as described above, 500 mg, 1.57 mmol) was dissolved in ethanol (5 ml) and water (2 ml) under an inert atmosphere. Tetrahydroxydiboron (739 mg, 7.83 mmol) was added portionwise at room temperature. The reaction mixture was stirred at 80° C. for 2 hours. Ice cold water (25 ml) was added and the product extracted with ethyl acetate (3×50 ml). The combined organic layer was dried over anhydrous sodium sulfate, filtrated and concentrated under reduced pressure. The obtained residue was washed with n-pentane to yield the title compound as a brown oil (350 mg). LCMS (method 1): 243/245 (M+H)⁺, Rt 0.20 min.

### Step 3: Preparation of 6-bromo-1H-1,7-naphthyridin-2-one (intermediate I-8)

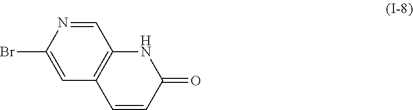 [SMILES: O=c1ccc2cc(Br)ncc2[nH]1]

(E)-3-(5-amino-2-bromo-4-pyridyl)prop-2-enoic acid (intermediate I-7 prepared as described above, 350 mg, 1.44 mmol) was dissolved in 1,4-dioxane (3.5 ml). Aqueous 5N hydrochloric acid (3.5 ml, 17.5 mmol) was added slowly and the mixture stirred at 95° C. for 16 hours. Water (10 ml) and aqueous saturated sodium bicarbonate (35 ml) were added and the product extracted with ethyl acetate (4×30 ml). The combined organic layers were dried over anhydrous sodium sulfate, filtered and concentrated under reduced pressure. The residue was washed with n-pentane (3×5 ml), then dried under high vacuum to obtain the title compound as a brown semi solid (130 mg). LCMS (method 1): 225/227 (M+H)⁺, Rt 0.82 min. ¹H NMR (400 MHz, DMSO-d₆) δ ppm 6.81 (d, J=9.54 Hz, 1H), 7.90 (d, J=9.54 Hz, 1H), 7.95 (s, 1H), 8.41 (s, 1H), 12.09 (br s, 1H).

### Step 4: Preparation of 6-bromo-1-(2,2,3,3,3-pentafluoropropyl)-1,7-naphthyridin-2-one (intermediate I-9)

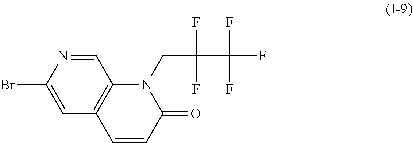 [SMILES: O=c1ccc2cc(Br)ncc2n1CC(F)(F)C(F)(F)F]

6-bromo-1H-1,7-naphthyridin-2-one (intermediate I-8 prepared as described above, 1.80 g, 8.00 mmol) was dissolved in tetrahydrofuran (18 ml) under an inert atmosphere. Potassium carbonate (4.42 g, 32 mmol) and 2,2,3,3,3-pentafluoropropyl trifluoromethansulfonate (5.47 ml, 9.31 g, 32 mmol) were added and the reaction was stirred at 75° C. for 4 hours. More potassium carbonate (4.42 g, 32 mmol) and 2,2,3,3,3-pentafluoropropyl trifluoromethansulfonate (5.47 ml, 9.31 g, 32 mmol) were added and stirring continued at 75° C. for 4 hours. After cooling to room temperature, water was added and the product extracted with ethyl acetate (6×200 ml). The combined organic layers were dried over anhydrous sodium sulfate, filtered and concentrated under reduced pressure. Purification of the crude material by flash chromatography over silica gel (gradient ethyl acetate in cyclohexane) afforded the desired product. (900 mg). LCMS (method 1): 357/359 (M+H)⁺, Rt 1.00 min. ¹H NMR (400 MHz, CDCl₃) δ ppm 5.02 (br s, 2H) 6.97 (d, 1H) 7.63-7.68 (m, 2H) 8.56 (s, 1H).

### Step 5: Preparation of 1-[5-fluoro-1-oxido-6-[2-oxo-1-(2,2,3,3,3-pentafluoropropyl)-1,7-naphthyridin-6-yl]pyridin-1-ium-3-yl]cyclopropanecarbonitrile (intermediate I-10)

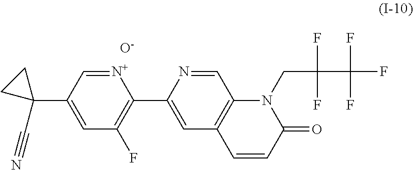

A solution of 1-(5-fluoro-1-oxido-pyridin-1-ium-3-yl)cyclopropanecarbonitrile (prepared as described in WO 20/182577, 2.20 g, 12 mmol) in tetrahydrofuran (22 ml) was degassed for 10 minutes. 2,2,6,6-Tetramethylpiperidinylzinc chloride lithium chloride complex (1.0 mol/L in THF, 19 ml, 19 mmol) was added dropwise at 0° C. and after addition stirring continued for an additional 15 minutes at 0° C. A solution of 6-bromo-1-(2,2,3,3,3-pentafluoropropyl)-1,7-naphthyridin-2-one (intermediate I-9 prepared as described above, 4.4 g, 12 mmol) in THE (44 ml) was prepared in a separate flask and degassed with nitrogen for 10 minutes. This solution was added to the first one at 10° C. [1,1′-Bis(diphenyl-phosphino)-ferrocene]dichloropalladium(II) (540 mg, 0.74 mmol) was added and the reaction mixture was heated to 60° C. for 16 hours. Aqueous saturated ammonium chloride (70 ml) was added and the product extracted three times with ethyl acetate. The combined organic layers were washed with water and brine, dried over anhydrous sodium sulfate, filtered and concentrated under reduced pressure. Purification of the crude material by flash chromatography over silica gel (gradient ethyl acetate in cyclohexane) afforded the desired product. (3.90 g). LCMS (method 1): 455 (M+H)⁺, Rt 0.99 min.

### Step 6: Preparation of 1-[5-ethylsulfanyl-1-oxido-6-[2-oxo-1-(2,2,3,3,3-pentafluoropropyl)-1,7-naphthyridin-6-yl]pyridin-1-ium-3-yl]cyclopropanecarbonitrile (intermediate I-11)

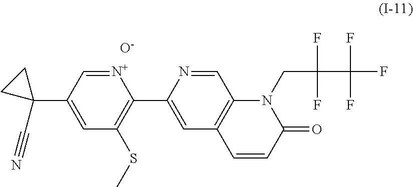

To a solution of 1-[5-fluoro-1-oxido-6-[2-oxo-1-(2,2,3,3,3-pentafluoropropyl)-1,7-naphthyridin-6-yl]pyridin-1-ium-3-yl]cyclopropanecarbonitrile (intermediate I-10 prepared as described above, 500 mg, 1.10 mmol) in N-methyl-2-pyrrolidon (10 ml) was added sodium ethanthiolate (174 mg, 1.65 mmol) at 0° C. under nitrogen atmosphere. The mixture was stirred at room temperature for 2 hours. Ice cold water (10 ml) was added and the product extracted three times with ethyl acetate. The combined organic layers were washed with water and brine, dried over anhydrous sodium sulfate, filtered and concentrated under reduced pressure. Purification of the crude material by flash chromatography over silica gel (gradient ethyl acetate in cyclohexane) afforded the desired product as a brown solid (320 mg). LCMS (method 1): 497 (M+H)⁺, Rt 1.01 min.

### Step 7: Preparation of 1-[5-ethylsulfanyl-6-[2-oxo-1-(2,2,3,3,3-pentafluoropropyl)-1,7-naphthyridin-6-yl]-3-pyridylicyclopropanecarbonitrile (compound P13)

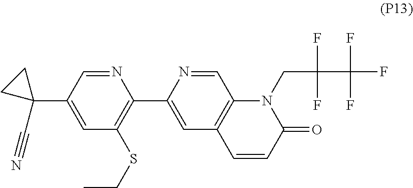 [SMILES: CCSc1cc(C2(C#N)CC2)cnc1-c1cc2ccc(=O)n(CC(F)(F)C(F)(F)F)c2cn1]

A solution of 1-[5-ethylsulfanyl-1-oxido-6-[2-oxo-1-(2,2,3,3,3-pentafluoropropyl)-1,7-naphthyridin-6-yl]pyridin-1-ium-3-yl]cyclopropanecarbonitrile (intermediate I-11 prepared as described above, 600 mg, 1.21 mmol) and 4,4,5,5-tetramethyl-2-(4,4,5,5-tetramethyl-1,3,2-dioxaborolan-2-yl)-1,3,2-dioxaborolane (307 mg, 1.21 mmol) in acetonitrile (12 ml) was stirred at 70° C. for 12 hours. The mixture was diluted with water (100 ml) and the product extracted with ethyl acetate (3×100 ml). The combined organic layers were washed with brine (50 ml), dried over anhydrous sodium sulfate and concentrated under reduced pressure. Purification of the crude material by flash chromatography over silica gel (gradient ethyl acetate in cyclohexane) afforded the desired product as a colorless solid (430 mg). LCMS (method 1): 481 (M+H)⁺, Rt 1.12 min. ¹H NMR (400 MHz, CDCl₃) δ ppm 1.38 (t, J=7.3 Hz, 3H) 1.52-1.58 (m, 2H) 1.85-1.90 (m, 2H) 2.99 (q, J=7.3 Hz, 2H) 5.10 (br s, 2H) 6.96 (d, J=9.7 Hz, 1H) 7.72 (d, J=2.1 Hz, 1H) 7.82 (d, J=9.5 Hz, 1H) 8.25-8.30 (m, 2H) 8.91 (s, 1H).

### Step 8: Preparation of 1-[5-ethylsulfonyl-6-[2-oxo-1-(2,2,3,3,3-pentafluoropropyl)-1,7-naphthyridin-6-yl]-3-pyridylicyclopropanecarbonitrile (title compound P3)

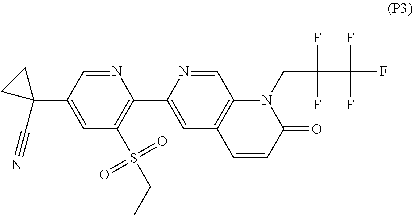 [SMILES: CCS(=O)(=O)c1cc(C2(C#N)CC2)cnc1-c1cc2ccc(=O)n(CC(F)(F)C(F)(F)F)c2cn1]

To a solution of 1-[5-ethylsulfanyl-6-[2-oxo-1-(2,2,3,3,3-pentafluoropropyl)-1,7-naphthyridin-6-yl]-3-pyridyl]cyclopropancarbonitrile (compound P13 prepared as described above, 430 mg, 0.90 mmol) in acetonitrile (8.6 ml) was added 3-chlorobenzenecarboperoxic acid (486 mg, 1.97 mmol) at 0° C. The reaction mixture was stirred at room temperature for 2 hours. It was then quenched with aqueous 2N sodium hydroxide (20 ml) and water (10 ml). The product was extracted with ethyl acetate (2×20 ml), the combined organic layers washed with brine (20 ml), dried over anhydrous sodium sulfate, filtered and concentrated under reduced pressure. Purification of the crude material by flash chromatography over silica gel (gradient ethyl acetate in cyclohexane) afforded the desired product as a colorless solid (300 mg), mp: 224-226° C. LCMS (method 1): 513 (M+H)⁺, Rt 1.07 min. ¹H NMR (400 MHz, DMSO-d₆) δ ppm 1.23-1.35 (t, 3H), 1.78-1.91 (m, 2H), 1.94-2.01 (m, 2H) 4.01 (q, 2H), 5.44 (br t, 2H), 7.05 (d, 1H) 8.22 (d, 1H) 8.24 (s, 1H) 8.37 (d, 1H) 8.90 (d, 1H) 9.11 (s, 1H).

### Example H3: Preparation of 6-[3-ethylsulfonyl-6-(1,2,4-triazol-1-yl)-2-pyridyl]-1-(2,2,3,3,3-pentafluoropropyl)-1,7-naphthyridin-2-one (compound P4)

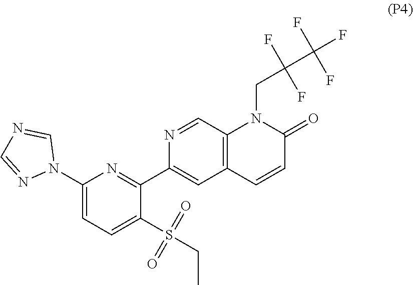 [SMILES: CCS(=O)(=O)c1ccc(-n2cncn2)nc1-c1cc2ccc(=O)n(CC(F)(F)C(F)(F)F)c2cn1]

### Step 1: Preparation of 6-(3-fluoro-1-oxido-pyridin-1-ium-2-yl)-1-(2,2,3,3,3-pentafluoropropyl)-1,7-naphthyridin-2-one (intermediate I-12)

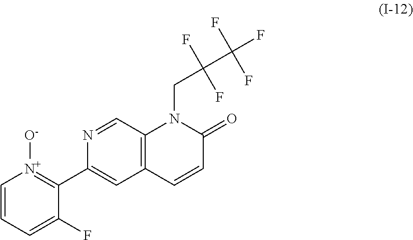 [SMILES: O=c1ccc2cc(-c3c(F)ccc[n+]3[O-])ncc2n1CC(F)(F)C(F)(F)F]

A solution 3-fluoro-1-oxido-pyridin-1ium (CAS: 695-37-4, 1.9 g, 17 mmol) in tetrahydrofuran (40 ml) was degassed for 10 minutes. 2,2,6,6-Tetramethylpiperidineylzinc chloride lithium chloride complex in tetrahydrofuran (1.0 mol/L, CAS: 1145881-09-9, 17 ml, 17 mmol) was added dropwise at 0° C. and after addition stirring continued for another 15 minutes at 0° C. A solution of 6-bromo-1-(2,2,3,3,3-pentafluoropropyl)-1,7-naphthyridin-2-one (intermediate I-9 prepared as described above, 4.0 g, 11 mmol) was dissolved in tetrahydrofuran (40 ml) and degassed. This solution was added to the first one, then [1,1′-bis(diphenylphosphino)-ferrocene]dichloropalladium(II) (490 mg, 0.67 mmol) was added and the reaction mixture was heated to 60° C. for 16 hours. Aqueous saturated sodium bicarbonate (80 ml) was added and the product extracted three times with ethyl acetate. The combined organic layers were washed with water and brine, dried over anhydrous sodium sulfate, filtered and concentrated under reduced pressure. Purification of the crude material by flash chromatography over silica gel (35% ethyl acetate in cyclohexane) afforded the desired product. (4.2 g). LCMS (method 1): 390 (M+H)⁺, Rt 0.94 min.

### Step 2: Preparation of 6-(3-ethylsulfanyl-1-oxido-pyridin-1-ium-2-yl)-1-(2,2,3,3,3-pentafluoropropyl)-1,7-naphthyridin-2-one (intermediate I-13)

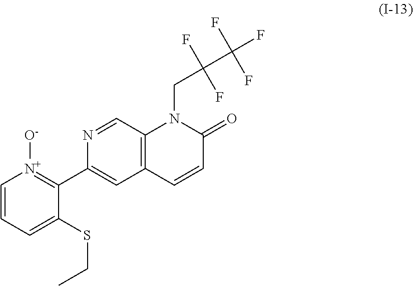 [SMILES: CCSc1ccc[n+]([O-])c1-c1cc2ccc(=O)n(CC(F)(F)C(F)(F)F)c2cn1]

To a solution of 6-(3-fluoro-1-oxido-pyridin-1-ium-2-yl)-1-(2,2,3,3,3-pentafluoropropyl)-1,7-naphthyridin-2-one (intermediate I-12 prepared as described above, 750 mg, 1.93 mmol) in dry N,N-dimethylformamide (11.25 ml) was added sodium ethanthiolate (446 mg, 4.24 mmol) at 0° C. The mixture was stirred at 0° C. for 15 minutes. Ice cold water (50 ml) was added and the product extracted three times with ethyl acetate. The combined organic layers were washed with water and brine, dried over anhydrous sodium sulfate, filtered and concentrated under reduced pressure to yield the title compound. (750 mg). LCMS (method 1): 432 (M+H)⁺, Rt 0.77 min.

### Step 3: Preparation of 6-(6-chloro-3-ethylsulfanyl-2-pyridyl)-1-(2,2,3,3,3-pentafluoropropyl)-1,7-naphthyridin-2-one (intermediate I-14)

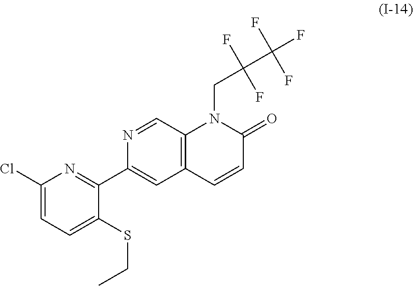 [SMILES: CCSc1ccc(Cl)nc1-c1cc2ccc(=O)n(CC(F)(F)C(F)(F)F)c2cn1]

6-(3-ethylsulfanyl-1-oxido-pyridin-1-ium-2-yl)-1-(2,2,3,3,3-pentafluoropropyl)-1,7-naphthyridin-2-one (intermediate I-13 prepared as described above, 3.0 g, 7.0 mmol) was dissolved in phosphoryl chloride (30 ml). The solution was stirred for 2 hours at room temperature. The reaction mixture was slowly poured on ice water, neutralized with aqueous saturated sodium bicarbonate and the product extracted with ethyl acetate (3×50 ml). The combined organic layers were dried over anhydrous sodium sulfate, filtered and concentrated under reduced pressure. Purification of the crude material by flash chromatography over silica gel (gradient ethyl acetate in cyclohexane) afforded the desired product. (1.2 g). LCMS (method 1): 450/452 (M+H)⁺, Rt 1.18 min. ¹H NMR (400 MHz, CDCl₃) δ ppm 1.37 (t, 3H) 2.96 (q, 2H) 5.12 (br s, 2H) 6.98 (d, 1H) 7.34 (d, 1H) 7.71 (d, 1H) 7.86 (d, 1H) 8.34 (s, 1H) 8.92 (s, 1H).

### Step 4: Preparation of 6-(6-chloro-3-ethylsulfonyl-2-pyridyl)-1-(2,2,3,3,3-pentafluoropropyl)-1,7-naphthyridin-2-one (intermediate I-15)

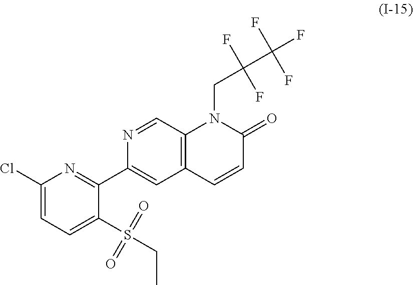 [SMILES: CCS(=O)(=O)c1ccc(Cl)nc1-c1cc2ccc(=O)n(CC(F)(F)C(F)(F)F)c2cn1]

To a solution of 6-(6-chloro-3-ethylsulfanyl-2-pyridyl)-1-(2,2,3,3,3-pentafluoropropyl)-1,7-naphthyridin-2-one (intermediate I-14 prepared as described above 1.2 g, 2.7 mmol) in trifluoromethylbenzene (12 ml) at 0° C. was added 3-chlorobenzencarboperoxoic acid (1.4 g, 5.9 mmol) portionwise. The mixture was stirred for 1 hour at room temperature, then poured on aqueous saturated sodium bicarbonate and the product extracted with ethyl acetate (3×80 ml). The combined organic layers were dried over anhydrous sodium sulfate, filtered and concentrated under reduced pressure. Purification of the crude material by flash chromatography over silica gel (gradient ethyl acetate in cyclohexane) afforded the desired product as a colorless solid. (1.1 g). LCMS (method 1): 482/484 (M+H)⁺, Rt 1.10 min. ¹H NMR (400 MHz, CDCl₃) δ ppm 1.43 (t, 3H) 3.97 (q, 2H) 5.13 (br s, 2H) 7.00 (d, 1H) 7.60 (d, 1H) 7.83 (d, 1H) 8.08-8.12 (m, 1H) 8.46 (d, 1H) 8.78 (s, 1H).

### Step 5: Preparation of 6-[3-ethylsulfonyl-6-(1,2,4-triazol-1-yl)-2-pyridyl]-1-(2,2,3,3,3-pentafluoropropyl)-1,7-naphthyridin-2-one (title compound P4)

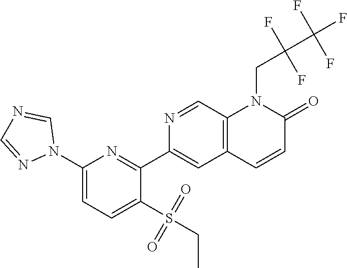 [SMILES: CCS(=O)(=O)c1ccc(-n2cncn2)nc1-c1cc2ccc(=O)n(CC(F)(F)C(F)(F)F)c2cn1]

To a solution of 6-(6-chloro-3-ethylsulfonyl-2-pyridyl)-1-(2,2,3,3,3-pentafluoropropyl)-1,7-naphthyridin-2-one (intermediate I-15 prepared as described above, 350 mg, 0.73 mmol) in acetonitrile (3.5 ml) were added 1H-1,2,4-triazole (75.3 mg, 1.09 mmol) and potassium carbonate (151 mg, 1.09 mmol). The reaction mixture was stirred at 90° C. for 4 hours, then diluted with water. The precipitated product was filtered off. Purification of the crude material by flash chromatography over silica gel (gradient methanol in ethyl acetate) afforded the desired product as a colorless solid (230 mg), mp: 314-316° C. LCMS (method 1): 515 (M+H)⁺, Rt 1.07 min. ¹H NMR (400 MHz, DMSO-d₆) δ ppm 1.30 (t, 3H) 4.04 (q, 2H) 5.46 (br s, 2H) 7.08 (d, 1H) 8.16 (d, 1H) 8.20 (br d, 1H) 8.44 (s, 1H) 8.50 (s, 1H) 8.70 (d, 1H) 9.15 (s, 1H) 9.65 (s, 1H).

### Example H4: Preparation of 6-(6-cyclopropyl-3-ethylsulfonyl-2-pyridyl)-1-(2,2,3,3,3-pentafluoropropyl)-1,7-naphthyridin-2-one (compound P5)

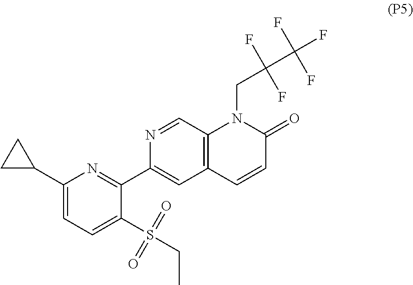 [SMILES: CCS(=O)(=O)c1ccc(C2CC2)nc1-c1cc2ccc(=O)n(CC(F)(F)C(F)(F)F)c2cn1]

To a solution of 6-(6-chloro-3-ethylsulfonyl-2-pyridyl)-1-(2,2,3,3,3-pentafluoropropyl)-1,7-naphthyridin-2-one (intermediate I-15 prepared as described above, 400 mg, 0.81 mmol) in toluene (8 ml) and water (1.2 ml) were added potassium carbonate (344 mg, 2.49 mmol) and cyclopropylboronic acid (188 mg, 2.08 mmol). The mixture was degassed with nitrogen for 10 minutes, then [1,1′-bis(diphenyl-phosphino)ferrocene]dichloro-palladium(II) (35 mg, 0.042 mmol) added. The reaction mixture was heated in the microwave at 110° C. for 1.5 hours, diluted with water and the product extracted three times with ethyl acetate. The combined organic layers were washed with water and brine, dried over anhydrous sodium sulfate, filtered and concentrated under reduced pressure. Purification of the crude material by flash chromatography over silica gel (gradient ethyl acetate in cyclohexane) afforded the desired product as a solid (210 mg), mp: 212-214° C. LCMS (method 1): 488 (M+H)⁺, Rt 1.16 min. ¹H NMR (400 MHz, DMSO-d₆) δ ppm 1.11-1.15 (m, 4H) 1.25 (t, 3H) 2.28-2.38 (m, 1H) 3.89 (q, 2H) 5.44 (br t, 2H) 7.03 (d, 1H) 7.66 (d, 1H) 8.16 (s, 1H) 8.21 (d, 1H) 8.27 (d, 1H) 9.07 (s, 1H).

### Example H5: Preparation of 2-[5-ethylsulfonyl-6-[2-oxo-1-(2,2,3,3,3-pentafluoropropyl)-1,7-naphthyridin-6-yl]-3-pyridyl]-2-methyl-propanenitrile (compound P6)

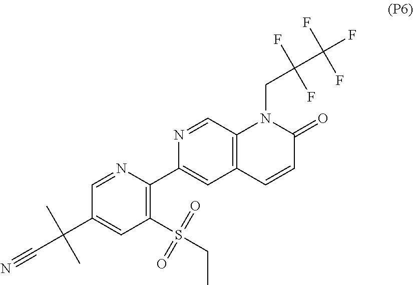 [SMILES: CCS(=O)(=O)c1cc(C(C)(C)C#N)cnc1-c1cc2ccc(=O)n(CC(F)(F)C(F)(F)F)c2cn1]

### Step 1: Preparation of 2-[5-fluoro-1-oxido-6-[2-oxo-1-(2,2,3,3,3-pentafluoropropyl)-1,7-naphthyridin-6-yl]pyridin-1-ium-3-yl]-2-methyl-propanenitrile (intermediate I-16)

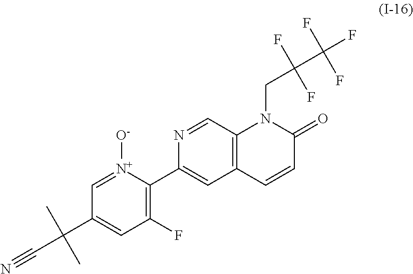 [SMILES: CC(C)(C#N)c1cc(F)c(-c2cc3ccc(=O)n(CC(F)(F)C(F)(F)F)c3cn2)[n+]([O-])c1]

A solution of 2-(5-fluoro-1-oxido-pyridin-1-ium-3-yl)-2-methyl-propanenitrile (prepared as described in WO 20/182577, 1.5 g, 8.4 mmol) in tetrahydrofuran (20 ml) was degassed for 10 minutes. To this was added 2,2,6,6-tetramethylpiperidineylzinc chloride lithium chloride complex in tetrahydrofuran (1 mmol/ml, CAS: 1145881-09-9, 8.4 ml, 8.4 mmol) dropwise at 0° C. over 15 minutes. In a separate flask a solution of 6-bromo-1-(2,2,3,3,3-pentafluoropropyl)-1,7-naphthyridin-2-one (intermediate I-9 prepared as described above, 2.0 g, 5.6 mmol) in tetrahydrofuran (20 ml) was degassed for 10 minutes with nitrogen. This solution was added to the first one at 10° C., then [1,1′-bis(diphenylphosphino)-ferrocene]dichloro-palladium(II) (250 mg, 0.34 mmol) added and the mixture heated to 60° C. for 16 hours. Aqueous saturated sodium bicarbonate (30 ml) was added and the product extracted three times with ethyl acetate. The combined organic layers were washed with water and brine, dried over anhydrous sodium sulfate, filtered and concentrated under reduced pressure. Purification of the crude material by flash chromatography over silica gel (35% ethyl acetate in cyclohexane) afforded the desired product as a solid. (2.2 g). LCMS (method 1): 457 (M+H)⁺, Rt 0.90 min.

### Step 2: Preparation of 2-[5-ethylsulfanyl-1-oxido-6-[2-oxo-1-(2,2,3,3,3-pentafluoropropyl)-1,7-naphthyridin-6-yl]pyridin-1-ium-3-yl]-2-methyl-propanenitrile (intermediate I-17)

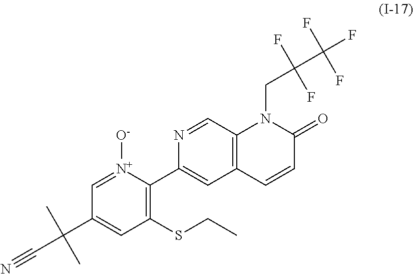 [SMILES: CCSc1cc(C(C)(C)C#N)c[n+]([O-])c1-c1cc2ccc(=O)n(CC(F)(F)C(F)(F)F)c2cn1]

To a solution of 2-[5-fluoro-1-oxido-6-[2-oxo-1-(2,2,3,3,3-pentafluoropropyl)-1,7-naphthyridin-6-yl]pyridin-1-ium-3-yl]-2-methyl-propanenitrile (intermediate I-16 prepared as described above, 1.20 g, 2.63 mmol) in dry N,N-dimethylformamide (18 ml) at 0° C. was added sodium ethanethiolate (608 mg. 5.79 mmol). The mixture was stirred at 0° C. for 15 minutes, then ice cold water (50 ml) added and the product extracted three times with ethyl acetate. The combined organic layers were washed with water and brine, dried over anhydrous sodium sulfate, filtered and concentrated under reduced pressure to yield the title compound. (1.1 g). LCMS (method 1): 499 (M+H)⁺, Rt 0.93 min.

### Step 3: Preparation of 2-[5-ethylsulfanyl-6-[2-oxo-1-(2,2,3,3,3-pentafluoropropyl)-1,7-naphthyridin-6-yl]-3-pyridyl]-2-methyl-propanenitrile (compound P17)

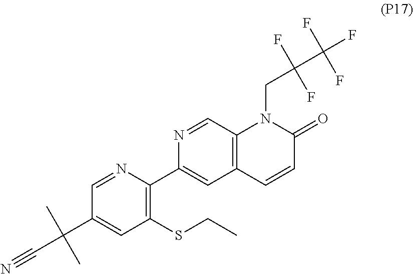 [SMILES: CCSc1cc(C(C)(C)C#N)cnc1-c1cc2ccc(=O)n(CC(F)(F)C(F)(F)F)c2cn1]

2-[5-ethylsulfanyl-1-oxido-6-[2-oxo-1-(2,2,3,3,3-pentafluoropropyl)-1,7-naphthyridin-6-yl]pyridin-1-ium-3-yl]-2-methyl-propanenitrile (intermediate I-17 prepared as described above, 1.1 g, 2.2 mmol) was dissolved in tetrahydrofuran (22 ml). Aqueous saturated ammonium chloride (11 ml) was added, followed by metallic zinc (290 mg, 4.4 mmol) at 0° C. The reaction mixture was stirred at room temperature for 16 hours, then water (40 ml) added and the product extracted with ethyl acetate (3×50 ml). The combined organic layers were dried over anhydrous sodium sulfate, filtered and concentrated under reduced pressure. Purification of the crude material by flash chromatography over silica gel (ethyl acetate in cyclohexane) afforded the desired product as a faint yellow solid (230 mg). LCMS (method 1): 484 (M+H)⁺, Rt 1.11 min.

### Step 4: Preparation of 2-[5-ethylsulfonyl-6-[2-oxo-1-(2,2,3,3,3-pentafluoropropyl)-1,7-naphthyridin-6-yl]-3-pyridyl]-2-methyl-propanenitrile (compound P6)

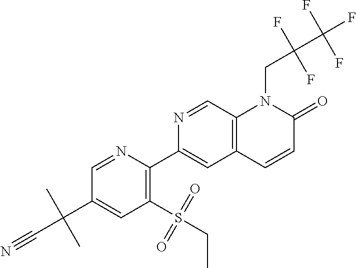 [SMILES: CCS(=O)(=O)c1cc(C(C)(C)C#N)cnc1-c1cc2ccc(=O)n(CC(F)(F)C(F)(F)F)c2cn1]

To a solution of 2-[5-ethylsulfanyl-6-[2-oxo-1-(2,2,3,3,3-pentafluoropropyl)-1,7-naphthyridin-6-yl]-3-pyridyl]-2-methyl-propanenitrile (compound P17 prepared as described above, 60 mg, 0.124 mmol) in acetonitrile (8 ml) at 0° C. was added 3-chlorobenzenecarboperoxoic acid (70%, 67 mg, 0.274 mmol).

The mixture was stirred for 2 hours at room temperature. Aqueous 2N sodium hydroxide (10 ml) and water (10 ml) were added and the product extracted with ethyl acetate (2×10 ml). The organic layer was washed with brine, dried over anhydrous sodium sulfate, filtered and concentrated under reduced pressure. Purification of the crude material by flash chromatography over silica gel (30% ethyl acetate in cyclohexane) afforded the desired product as a colorless solid (45 mg), mp: 245-247° C. LCMS (method 1): 515 (M+H)⁺, Rt 1.02 min. ¹H NMR (400 MHz, CDCl₃) δ ppm 1.44 (t, 3H) 1.89 (s, 6H) 3.99 (q, 2H) 5.13 (br s, 2H) 6.99 (d, 1H) 7.83 (d, 1H) 8.09 (s, 1H) 8.53 (d, 1H) 8.78 (s, 1H) 9.09 (d, 1H).

### Example H6: Preparation of N-[5-ethylsulfonyl-6-[2-oxo-1-(2,2,3,3,3-pentafluoropropyl)-1,7-naphthyridin-6-yl]-3-pyridyl]-N-methyl-acetamide (compound P7)

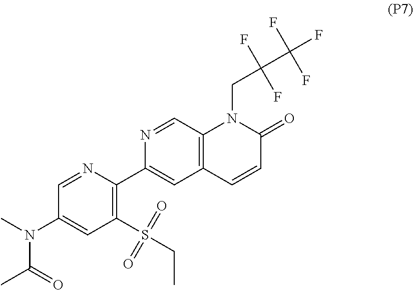 [SMILES: CCS(=O)(=O)c1cc(N(C)C(C)=O)cnc1-c1cc2ccc(=O)n(CC(F)(F)C(F)(F)F)c2cn1]

Step 1: Preparation of tert-butyl N-[5-fluoro-1-oxido-6-[2-oxo-1-(2,2,3,3,3-pentafluoropropyl)-1,7-naphthyridin-6-yl]pyridin-1-ium-3-yl]-N-methyl-carbamate (intermediate I-18)

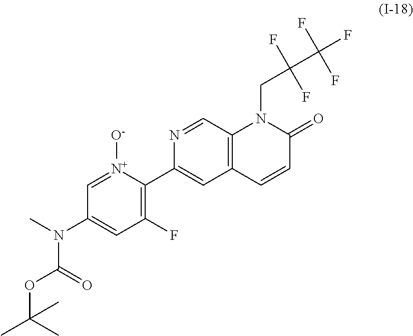 [SMILES: CN(C(=O)OC(C)(C)C)c1cc(F)c(-c2cc3ccc(=O)n(CC(F)(F)C(F)(F)F)c3cn2)[n+]([O-])c1]

A solution of tert-butyl N-(5-fluoro-1-oxido-pyridin-1-ium-3-yl)-N-methyl-carbamate (prepared as described in WO 20/182577, 3.0 g, 12 mmol) in tetrahydrofuran (30 ml) was degassed with nitrogen for 10 minutes and cooled to 0° C. 2,2,6,6-Tetramethylpiperidineylzinc chloride lithium chloride complex in tetrahydrofuran (1 mmol/ml, CAS: 1145881-09-9, 19 ml, 19 mmol) was added dropwise. In another flask, a solution of 6-bromo-1-(2,2,3,3,3-pentafluoropropyl)-1,7-naphthyridin-2-one (intermediate I-9 prepared as described above, 3.1 g, 8.7 mmol) in tetrahydrofuran (30 ml) was degassed with nitrogen, then both solution were mixed and [1,1′-bis(diphenylphosphino)-ferrocene]dichloro-palladium(II) (540 mg, 0.74 mmol) was added and the mixture heated to 60° C. for 16 hours. Aqueous saturated ammonium chloride (70 ml) was added and the product extracted three times with ethyl acetate. The combined organic layers were washed with water and brine, dried over anhydrous sodium sulfate, filtered and concentrated under reduced pressure. Purification of the crude material by flash chromatography over silica gel (65% ethyl acetate in cyclohexane) afforded the desired product as a solid (4.3 g). LCMS (method 1): 519 (M+H)⁺, Rt 1.04 min. ¹H NMR (400 MHz, CDCl₃) δ ppm 1.55 (s, 9H) 3.34 (s, 3H) 4.85-5.33 (m, 2H) 6.98 (d, 1H) 7.38-7.55 (m, 1H) 7.80 (d, 1H) 8.11-8.17 (m, 1H) 8.36 (s, 1H) 8.96 (s, 1H).

### Step 2: Preparation of tert-butyl N-[5-ethylsulfanyl-1-oxido-6-[2-oxo-1-(2,2,3,3,3-pentafluoropropyl)-1,7-naphthyridin-6-yl]pyridin-1-ium-3-yl]-N-methyl-carbamate (intermediate I-19)

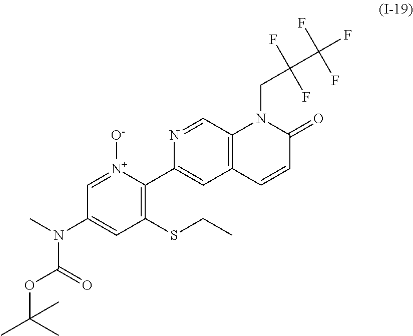 [SMILES: CCSc1cc(N(C)C(=O)OC(C)(C)C)c[n+]([O-])c1-c1cc2ccc(=O)n(CC(F)(F)C(F)(F)F)c2cn1]

To a solution tert-butyl N-[5-fluoro-1-oxido-6-[2-oxo-1-(2,2,3,3,3-pentafluoropropyl)-1,7-naphthyridin-6-yl]pyridin-1-ium-3-yl]-N-methyl-carbamate (intermediate I-18 prepared as described above, 4.30 g, 8.30 mmol) in dry N-methyl pyrrolidone (21.5 ml) at 0° C. was added sodium ethanethiolate (1.05 g, 12.44 mmol). The mixture was stirred at room temperature for 2 hours, ice cold water (10 ml) added and the product extracted three times with ethyl acetate. The combined organic layers were washed with water and brine, dried over anhydrous sodium sulfate, filtered and concentrated under reduced pressure. Purification of the crude material by flash chromatography over silica gel (5% methanol in ethyl acetate) afforded the desired product as a semi solid (3.0 g). LCMS (method 1): 561 (M+H)⁺, Rt 1.10 min. ¹H NMR (400 MHz, CDCl₃) δ ppm 1.30 (t, 3H) 1.53 (s, 9H) 2.88 (q, 2H) 3.31 (s, 3H) 4.82-5.32 (m, 2H) 6.95 (d, 1H) 7.29-7.43 (m, 1H) 7.77 (d, 1H) 7.95 (s, 1H) 8.17 (d, 1H) 8.93 (s, 1H).

### Step 3: Preparation of tert-butyl N-[5-ethylsulfanyl-6-[2-oxo-1-(2,2,3,3,3-pentafluoropropyl)-1,7-naphthyridin-6-yl]-3-pyridyl]-N-methyl-carbamate (intermediate I-20)

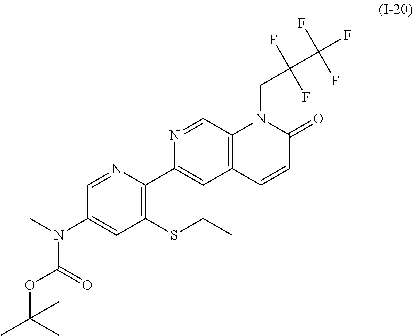 [SMILES: CCSc1cc(N(C)C(=O)OC(C)(C)C)cnc1-c1cc2ccc(=O)n(CC(F)(F)C(F)(F)F)c2cn1]

To a solution of tert-butyl N-[5-ethylsulfanyl-1-oxido-6-[2-oxo-1-(2,2,3,3,3-pentafluoropropyl)-1,7-naphthyridin-6-yl]pyridin-1-ium-3-yl]-N-methyl-carbamate (intermediate I-19 prepared as described above, 2.90 g, 5.17 mmol) in acetonitrile (29 ml) was added 4,4,5,5-tetramethyl-2-(4,4,5,5-tetramethyl-1,3,2-dioxaborolan-2-yl)-1,3,2-dioxaborolane (1.58 g, 6.21 mmol). The mixture was stirred at 60° C. for 16 hours, diluted with water (100 ml) and the product extracted with ethyl acetate (3×100 ml). The combined organic layers were washed with brine (50 ml), dried over anhydrous sodium sulfate, filtered and concentrated under reduced pressure. Purification of the crude material by flash chromatography over silica gel (25% ethyl acetate in cyclohexane) afforded the desired product as a colorless solid (2.2 g), mp: 172-174° C. LCMS (method 1): 545 (M+H)⁺, Rt 1.24 min. ¹H NMR (400 MHz, CDCl₃) δ ppm 1.38 (t, 3H) 1.52 (s, 9H) 2.95 (q, 2H) 3.37 (s, 3H) 5.12 (br s, 2H) 6.96 (d, 1H) 7.67 (s, 1H) 7.83 (d, 1H) 8.25 (s, 1H) 8.39 (d, 1H) 8.92 (s, 1H).

### Step 4: Preparation of tert-butyl N-[5-ethylsulfonyl-6-[2-oxo-1-(2,2,3,3,3-pentafluoropropyl)-1,7-naphthyridin-6-yl]-3-pyridyl]-N-methyl-carbamate (intermediate I-21)

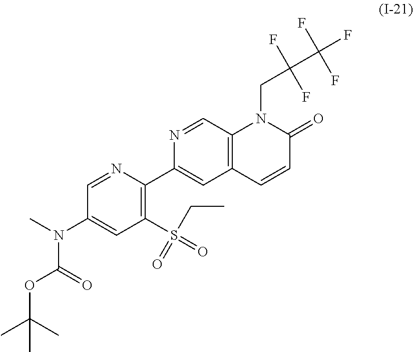 [SMILES: CCS(=O)(=O)c1cc(N(C)C(=O)OC(C)(C)C)cnc1-c1cc2ccc(=O)n(CC(F)(F)C(F)(F)F)c2cn1]

To a solution of tert-butyl N-[5-ethylsulfanyl-6-[2-oxo-1-(2,2,3,3,3-pentafluoropropyl)-1,7-naphthyridin-6-yl]-3-pyridyl]-N-methyl-carbamate (intermediate I-20 prepared as described above, 1.10 g, 2.02 mmol) in acetonitrile (22 ml) at 0° C. was added 3-chlorobenzenecarboperoxoic acid (70%, 1.10 g, 4.44 mmol). The mixture was stirred at room temperature for 2 hours, then aqueous 2N sodium hydroxide (20 ml) and water (20 ml) were added. The product was extracted with ethyl acetate (2×50 ml), the combined organic layers washed with brine (50 ml), dried over anhydrous sodium sulfate, filtered and concentrated under reduced pressure. Purification of the crude material by flash chromatography over silica gel (30% ethyl acetate in cyclohexane) afforded the desired product as a colorless solid (870 mg), mp: 196-198° C. LCMS (method 1): 577 (M+H)⁺, Rt 1.13 min. ¹H NMR (400 MHz, CDCl₃) δ ppm 1.45 (t, 3H) 1.54 (m, 9H) 3.42 (s, 3H) 3.98 (q, 2H) 5.11 (br s, 2H) 6.97 (d, 1H) 7.81 (d, 1H) 8.06 (s, 1H) 8.42 (d, 1H) 8.77 (s, 1H) 8.88 (d, 1H).

### Step 5: Preparation of 6-[3-ethylsulfonyl-5-(methylamino)-2-pyridyl]-1-(2,2,3,3,3-pentafluoropropyl)-1,7-naphthyridin-2-one (intermediate I-22)

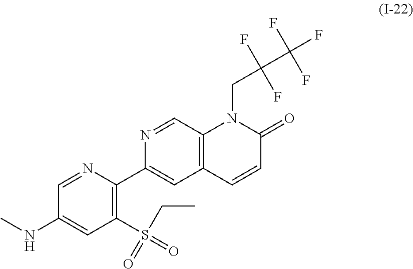 [SMILES: CCS(=O)(=O)c1cc(NC)cnc1-c1cc2ccc(=O)n(CC(F)(F)C(F)(F)F)c2cn1]

To a solution of tert-butyl N-[5-ethylsulfonyl-6-[2-oxo-1-(2,2,3,3,3-pentafluoropropyl)-1,7-naphthyridin-6-yl]-3-pyridyl]-N-methyl-carbamate (intermediate I-21 prepared as described above, 610 mg, 1.06 mmol) in benzotrifluoride (12.2 ml) at 0° C. was added trifluoroacetic acid (2.54 g, 1.70 ml, 21.16 mmol). The mixture was stirred at room temperature for 16 hours, then aqueous saturated sodium bicarbonate was added and the product extracted with ethyl acetate (3×50 ml). The combined organic layers were washed with brine, dried over anhydrous sodium sulfate, filtered und concentrated under reduced pressure. Purification of the crude material by flash chromatography over silica gel (ethyl acetate in cyclohexane) afforded the desired product as a pale-yellow solid (470 mg), mp: 224-226° C. LCMS (method 1): 477 (M+H)⁺, Rt 1.04 min. ¹H NMR (400 MHz, DMSO-d₆) δ ppm 1.26 (t, 3H) 2.84 (d, 3H) 3.96 (q, 2H) 5.42 (br t, 2H) 6.74-6.83 (m, 1H) 7.00 (d, 1H) 7.49 (d, 1H) 8.09 (s, 1H) 8.18 (d, 1H) 8.24 (d, 1H) 9.00 (s, 1H).

### Step 6: Preparation of N-[5-ethylsulfonyl-6-[2-oxo-1-(2,2,3,3,3-pentafluoropropyl)-1,7-naphthyridin-6-yl]-3-pyridyl]-N-methyl-acetamide (compound P7)

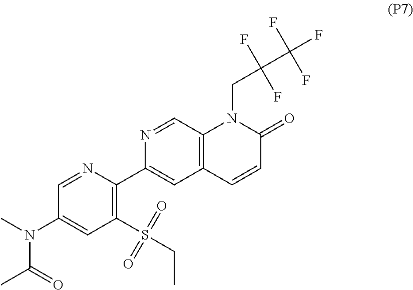 [SMILES: CCS(=O)(=O)c1cc(N(C)C(C)=O)cnc1-c1cc2ccc(=O)n(CC(F)(F)C(F)(F)F)c2cn1]

To a solution of 6-[3-ethylsulfonyl-5-(methylamino)-2-pyridyl]-1-(2,2,3,3,3-pentafluoropropyl)-1,7-naphthyridin-2-one (intermediate I-22 prepared as described above, 300 mg, 0.63 mmol) in pyridine (6 ml) were added N,N-dimethylpyridin-4-amine (15.5 mg, 0.126 mmol), followed by acetyl chloride (99 mg, 90 μl, 0.099 mmol). The mixture was stirred at room temperature for 4 hours, then water was added and the product was extracted with ethyl acetate (2×50 ml). The combined organic layers were dried over anhydrous sodium sulfate, filtered and concentrated under reduced pressure. Purification of the crude material by flash chromatography over silica gel (80% ethyl acetate in cyclohexane) afforded the desired product as a colorless solid (250 mg), mp: 280-282° C. LCMS (method 1): 519 (M+H)⁺, Rt 0.99 min. ¹H NMR (400 MHz, CDCl₃) δ ppm 1.46 (t, 3H) 2.09 (br s, 3H) 3.47 (br s, 3H) 4.03 (q, 2H) 4.90-5.32 (m, 2H) 7.01 (d, 1H) 7.84 (d, 1H) 8.12 (s, 1H) 8.41 (d, 1H) 8.80 (s, 1H) 8.85 (br s, 1H).

### Example H7: Preparation of 6-[5-(3-chloropyrazol-1-yl)-3-ethylsulfonyl-2-pyridyl]-1-(2,2,3,3,3-pentafluoropropyl)-1,7-naphthyridin-2-one (compound P8)

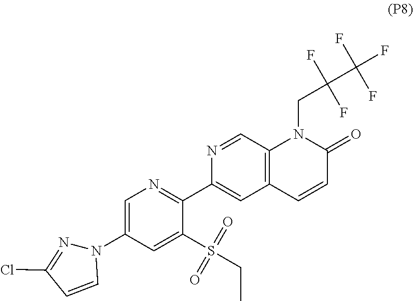 [SMILES: CCS(=O)(=O)c1cc(-n2ccc(Cl)n2)cnc1-c1cc2ccc(=O)n(CC(F)(F)C(F)(F)F)c2cn1]

### Step 1: Preparation of (3-chloropyrazol-1-yl)-5-fluoro-1-oxido-pyridin-1-ium (intermediate I-23)

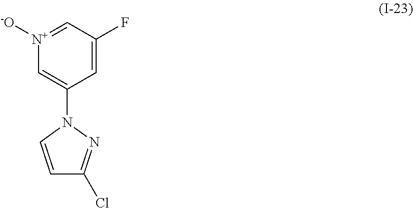

To a solution of 3-(3-chloropyrazol-1-yl)-5-fluoro-pyridine (CAS: 1374320-30-5, 1.4 g, 7.1 mmol) in dichloromethane (40 ml) at 0° C. was added 3-chloroperoxybenzoic acid (70%, 3.8 g, 16 mmol) portionwise. The mixture was warmed to room temperature and stirred for 16 hours. Aqueous saturated sodium bicarbonate was added and the product extracted with ethyl acetate (2×100 ml). The combined organic layer was washed with brine (70 ml), dried over anhydrous sodium sulfate, filtered and concentrated under reduced pressure. Purification of the crude material by flash chromatography over silica gel (80% ethyl acetate in cyclohexane) afforded the desired product as an off white solid (1.2 g). LCMS (method 1): 214/216 (M+H)⁺, Rt 0.58 min. ¹H NMR (400 MHz, CDCl₃) δ ppm 6.53 (d, 1H) 7.60 (br d, 1H) 7.86-7.95 (m, 1H) 8.17 (br s, 1H) 8.65 (s, 1H).

### Step 2: Preparation of 6-[5-(3-chloropyrazol-1-yl)-3-fluoro-1-oxido-pyridin-1-ium-2-yl]-1-(2,2,3,3,3-pentafluoropropyl)-1,7-naphthyridin-2-one (intermediate I-24)

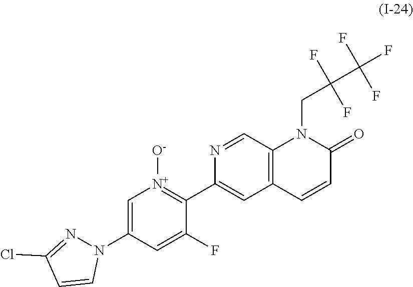 [SMILES: O=c1ccc2cc(-c3c(F)cc(-n4ccc(Cl)n4)c[n+]3[O-])ncc2n1CC(F)(F)C(F)(F)F]

A solution of (3-chloropyrazol-1-yl)-5-fluoro-1-oxido-pyridin-1-ium (intermediate I-23 prepared as describe above, 1.5 g, 7.0 mmol) in tetrahydrofuran (18 ml) was degassed with nitrogen for 10 minutes and cooled to 10° C. 2,2,6,6-Tetramethylpiperidineylzinc chloride lithium chloride complex in tetrahydrofuran (1 mmol/ml, CAS: 1145881-09-9, 7 ml, 7 mmol) was added dropwise. In another flask, a solution of 6-bromo-1-(2,2,3,3,3-pentafluoropropyl)-1,7-naphthyridin-2-one (intermediate I-9, prepared as described above, 1.8 g, 4.6 mmol) in tetrahydrofuran (18 ml) was degassed with nitrogen, then both solutions were mixed and [1,1′-bis(diphenylphosphino)ferrocene]dichloro-palladium(II) (230 mg, 0.30 mmol) added. The reaction mixture was heated to 60° C. for 15 hours. Aqueous saturated sodium bicarbonate (60 ml) was added and the product extracted three times with ethyl acetate (3×60 ml). The combined organic layers were dried over anhydrous sodium sulfate, filtered and concentrated under reduced pressure. Purification of the crude material by flash chromatography over silica gel (10% methanol in ethyl acetate) afforded the desired product as a solid (2.0 g). LCMS (method 1): 490/492 (M+H)⁺, Rt 1.07 min.

### Step 3: Preparation of 6-[5-(3-chloropyrazol-1-yl)-3-fluoro-2-pyridyl]-1-(2,2,3,3,3-pentafluoropropyl)-1,7-naphthyridin-2-one (intermediate I-25)

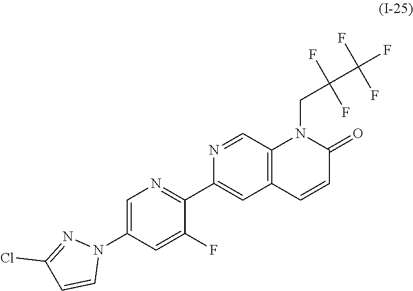 [SMILES: O=c1ccc2cc(-c3ncc(-n4ccc(Cl)n4)cc3F)ncc2n1CC(F)(F)C(F)(F)F]

To a solution of 6-[5-(3-chloropyrazol-1-yl)-3-fluoro-1-oxido-pyridin-1-ium-2-yl]-1-(2,2,3,3,3-pentafluoropropyl)-1,7-naphthyridin-2-one (intermediate I-24 prepared as described above, 1.56 g, 3.18 mmol) in dry acetonitrile (15.6 ml) was added 4,4,5,5-tetramethyl-2-(4,4,5,5-tetramethyl-1,3,2-dioxaborolan-2-yl)-1,3,2-dioxaborolane (809 mg, 3.18 mmol). The reaction mixture was stirred at 70° C. for 10 hours. Another batch of 4,4,5,5-tetramethyl-2-(4,4,5,5-tetramethyl-1,3,2-dioxaborolan-2-yl)-1,3,2-dioxaborolane (404 mg, 1.59 mmol) was added and stirring continued at 70° C. for 9 hours. The mixture was diluted with water (100 ml) and the product extracted with ethyl acetate (3×100 ml). The combined organic layers were washed with brine (50 ml), dried over anhydrous sodium sulfate and concentrated under reduced pressure. Purification of the crude material by flash chromatography over silica gel (0 to 40% ethyl acetate in cyclohexane) afforded the desired product as a pink solid (967 mg). LCMS (method 1): 474/476 (M+H)⁺, Rt 1.11 min. ¹H NMR (400 MHz, CDCl₃) δ ppm 5.12 (br s, 2H) 6.53 (d, 1H) 7.00 (d, 1H) 7.85 (d, 1H) 8.01 (m, 2H) 8.28 (s, 1H) 8.91 (d, 1H) 9.00 (s, 1H).

### Step 4: Preparation of 6-[5-(3-chloropyrazol-1-yl)-3-ethylsulfanyl-2-pyridyl]-1-(2,2,3,3,3-pentafluoropropyl)-1,7-naphthyridin-2-one (compound P14)

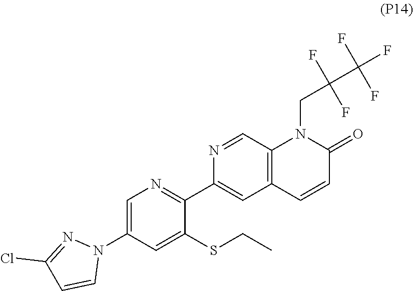 [SMILES: CCSc1cc(-n2ccc(Cl)n2)cnc1-c1cc2ccc(=O)n(CC(F)(F)C(F)(F)F)c2cn1]

To a solution of 6-[5-(3-chloropyrazol-1-yl)-3-fluoro-2-pyridyl]-1-(2,2,3,3,3-pentafluoropropyl)-1,7-naphthyridin-2-one (intermediate I-25 prepared as described above, 950 mg, 2.0 mmol) in N,N-dimethylformamide (10 ml) was added sodium ethanethiolate (250 mg. 3.0 mmol). The mixture was stirred at room temperature for 7 hours, then ice cold water (50 ml) was added and the product extracted three times with ethyl acetate (3×100 ml). The combined organic layers were washed with brine (50 ml), dried over anhydrous sodium sulfate, filtered and concentrated under reduced pressure. Purification of the crude material by flash chromatography over silica gel (40% ethyl acetate in cyclohexane) afforded the desired product as a colorless solid (700 mg), mp: 231-232° C. LCMS (method 1): 516/518 (M+H)⁺, Rt 1.20 min. ¹H NMR (400 MHz, CDCl₃) δ ppm 1.41 (t, 3H) 3.05 (q, 2H) 5.11 (br s, 2H) 6.50 (d, 1H) 6.97 (d, 1H) 7.84 (d, 1H) 7.98 (d, 1H) 8.09 (d, 1H) 8.31 (s, 1H) 8.67 (d, 1H) 8.93 (s, 1H).

### Step 5: Preparation of 6-[5-(3-chloropyrazol-1-yl)-3-ethylsulfonyl-2-pyridyl]-1-(2,2,3,3,3-pentafluoropropyl)-1,7-naphthyridin-2-one (compound P8)

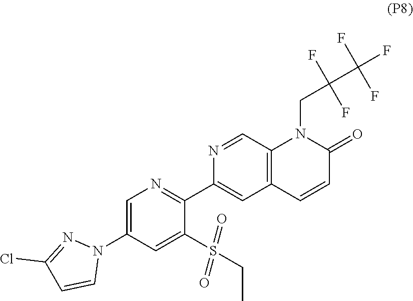 [SMILES: CCS(=O)(=O)c1cc(-n2ccc(Cl)n2)cnc1-c1cc2ccc(=O)n(CC(F)(F)C(F)(F)F)c2cn1]

To a solution of 6-[5-(3-chloropyrazol-1-yl)-3-ethylsulfanyl-2-pyridyl]-1-(2,2,3,3,3-pentafluoropropyl)-1,7-naphthyridin-2-one (compound P14 prepared as described above, 672 mg, 1.303 mmol) in benzotrifluoride (20 ml) at 0° C. was added 3-chlorobenzenecarboperoxoic acid (70%, 707 mg, 2.87 mmol). The mixture was stirred at room temperature for 5 hours, then ice cold water (50 ml) was added and the solution basified with aqueous 2N sodium hydroxide. The product was extracted with ethyl acetate (3×60 ml), the combined organic layers washed with water (100 ml) and brine (50 ml), dried over anhydrous sodium sulfate, filtered and concentrated under reduced pressure. Purification of the crude material by flash chromatography over silica gel (0 to 100% ethyl acetate in cyclohexane) afforded the desired product as a colorless solid (504 mg). LCMS (method 1): 548/550 (M+H)⁺, Rt 1.13 min. ¹H NMR (400 MHz, CDCl₃) δ ppm 1.47 (t, 3H) 4.05 (q, 2H) 5.13 (br s, 2H) 6.56 (d, 1H) 6.99 (d, 1H) 7.84 (d, 1H) 8.03-8.09 (m, 1H) 8.12 (s, 1H) 8.72 (d, 1H) 8.79 (s, 1H) 9.30 (d, 1H).

### Example H8. Preparation of 6-[3-ethylsulfonyl-5-(2-pyridyloxy)-2-pyridyl]-1-(2,2,3,3,3-pentafluoropropyl)-1,7-naphthyridin-2-one (compound P9)

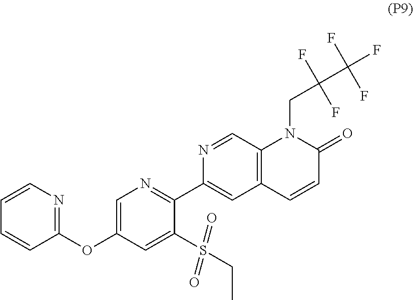 [SMILES: CCS(=O)(=O)c1cc(Oc2ccccn2)cnc1-c1cc2ccc(=O)n(CC(F)(F)C(F)(F)F)c2cn1]

### Step 1: Preparation of 6-[3-fluoro-1-oxido-5-(2-pyridyloxy)pyridin-1-ium-2-yl]-1-(2,2,3,3,3-Pentafluoropropyl)-1,7-naphthyridin-2-one (intermediate I-26)

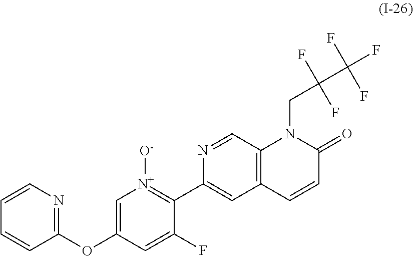 [SMILES: O=c1ccc2cc(-c3c(F)cc(Oc4ccccn4)c[n+]3[O-])ncc2n1CC(F)(F)C(F)(F)F]

A solution of 3-fluoro-1-oxido-5-(2-pyridyloxy)pyridin-1-ium (prepared as described in WO 20/182577, 3.0 g, 15.0 mmol) in tetrahydrofuran (30 ml) was degassed with nitrogen for 10 minutes and cooled to 0° C. 2,2,6,6-Tetramethylpiperidineylzinc chloride lithium chloride complex in tetrahydrofuran (1 mmol/ml, CAS: 1145881-09-9, 22 ml, 22 mmol) was added dropwise and the mixture stirred for 15 minutes at 0° C. In another flask, a solution of 6-bromo-1-(2,2,3,3,3-pentafluoropropyl)-1,7-naphthyridin-2-one (intermediate I-9 prepared as described above, 4.2 g, 12 mmol) in tetrahydrofuran (30 ml) was prepared and degassed with nitrogen, then both solutions were mixed and [1,1′-bis(diphenylphosphino)ferrocene]dichloro-palladium(II) (640 mg, 0.87 mmol) added. The reaction mixture was heated to 60° C. for 16 hours, then aqueous saturated sodium bicarbonate (30 ml) was added and the product extracted three times with ethyl acetate. The organic layer was washed with water and brine, dried over anhydrous sodium sulfate, filtered and concentrated under reduced pressure. Purification of the crude material by flash chromatography over silica gel (35% ethyl acetate in cyclohexane) afforded the desired product as a solid (4.0 g). LCMS (method 1): 483 (M+H)⁺, Rt 1.00 min.

### Step 2: Preparation of 6-[3-ethylsulfanyl-1-oxido-5-(2-pyridyloxy)pyridin-1-ium-2-yl]-1-(2,2,3,3,3-pentafluoropropyl)-1,7-naphthyridin-2-one (intermediate I-27)

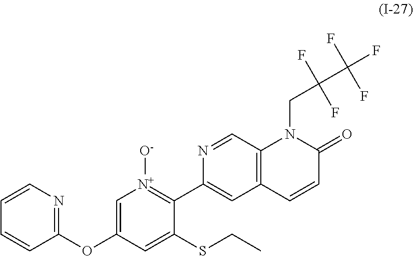 [SMILES: CCSc1cc(Oc2ccccn2)c[n+]([O-])c1-c1cc2ccc(=O)n(CC(F)(F)C(F)(F)F)c2cn1]

To a solution of 6-[3-fluoro-1-oxido-5-(2-pyridyloxy)pyridin-1-ium-2-yl]-1-(2,2,3,3,3-pentafluoropropyl)-1,7-naphthyridin-2-one (intermediate I-26 prepared as described above, 500 mg, 1.04 mmol) in dry N-methylpyrrolidone (10 ml) at 0° C. was added sodium ethanethiolate (164 mg. 1.55 mmol). The mixture was stirred at room temperature for 5 hours. More sodium ethanthiolate (164 mg. 1.55 mmol) was added and stirring continued at room temperature for 16 hours. Ice cold water (10 ml) was added and the product extracted three times with ethyl acetate. The combined organic layers were washed with water and brine, dried over anhydrous sodium sulfate, filtered and concentrated under reduced pressure. Purification of the crude material by flash chromatography over silica gel (4% methanol in ethyl acetate) afforded the desired product as a brown thick oil (300 mg). LCMS (method 1): 525 (M+H)⁺, Rt 1.03 min.

### Step 3: Preparation of 6-[3-ethylsulfanyl-5-(2-pyridyloxy)-2-pyridyl]-1-(2,2,3,3,3-pentafluoropropyl)-1,7-naphthyridin-2-one (compound P16)

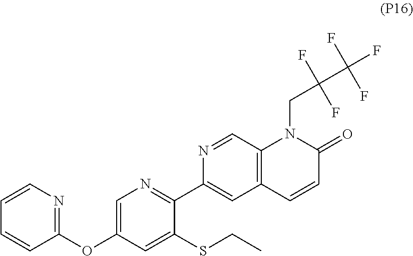 [SMILES: CCSc1cc(Oc2ccccn2)cnc1-c1cc2ccc(=O)n(CC(F)(F)C(F)(F)F)c2cn1]

To a solution of 6-[3-ethylsulfanyl-1-oxido-5-(2-pyridyloxy)pyridin-1-ium-2-yl]-1-(2,2,3,3,3-pentafluoropropyl)-1,7-naphthyridin-2-one (intermediate I-27 prepared as described above, 5.2 g, 9.91 mmol) in dry acetonitrile (104 ml) was added 4,4,5,5-tetramethyl-2-(4,4,5,5-tetramethyl-1,3,2-dioxaborolan-2-yl)-1,3,2-dioxaborolane (2.52 g, 9.91 mmol). The reaction mixture was stirred at 60° C. for 16 hours, diluted with water (100 ml) and the product extracted with ethyl acetate (3×100 ml). The combined organic layers were washed with brine (50 ml), dried over anhydrous sodium sulfate, filtered and concentrated under reduced pressure. Purification of the crude material by flash chromatography over silica gel (65% ethyl acetate in cyclohexane) afforded the desired product as a colorless solid (1.8 g). LCMS (method 1): 509 (M+H)⁺, Rt 1.17 min. ¹H NMR (400 MHz, CDCl₃) δ ppm 1.37 (t, 3H) 2.92 (q, 2H) 5.13 (br s, 2H) 6.96 (d, 1H) 7.07 (d, 1H) 7.11 (ddd, 1H) 7.58 (d, 1H) 7.80 (ddd, 1H) 7.84 (d, 1H) 8.22 (d, 1H) 8.28 (s, 1H) 8.36 (d, 1H) 8.93 (s, 1H).

### Step 4: Preparation of 6-[3-ethylsulfonyl-5-(2-pyridyloxy)-2-pyridyl]-1-(2,2,3,3,3-pentafluoropropyl)-1,7-naphthyridin-2-one (compound P9)

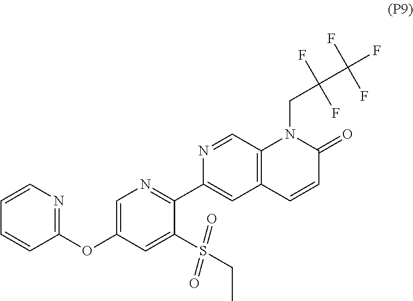 [SMILES: CCS(=O)(=O)c1cc(Oc2ccccn2)cnc1-c1cc2ccc(=O)n(CC(F)(F)C(F)(F)F)c2cn1]

To a solution of 6-[3-ethylsulfanyl-5-(2-pyridyloxy)-2-pyridyl]-1-(2,2,3,3,3-pentafluoropropyl)-1,7-naphthyridin-2-one (compound P16 prepared as described above, 350 mg, 0.688 mmol) in acetonitrile (7 ml) at 0° C. was added 3-chlorobenzenecarboperoxoic acid (70%, 373 mg, 1.51 mmol). The mixture was stirred for 2 hours at room temperature. Ice cold water (10 ml) was added and the solution basified with aqueous 2N sodium hydroxide (10 ml). The product was extracted with ethyl acetate (2×50 ml), the combined organic layers washed brine (20 ml), dried over anhydrous sodium sulfate, filtered and concentrated under reduced pressure. Purification of the crude material by flash chromatography over silica gel (30% ethyl acetate in cyclohexane) afforded the desired product as a colorless solid (195 mg), mp: 216-218° C. LCMS (method 1): 541 (M+H)⁺, Rt 1.11 min. ¹H NMR (400 MHz, CDCl₃) δ ppm 1.46 (t, 3H) 4.00 (q, 2H) 5.14 (br s, 2H) 6.99 (d, 1H) 7.12-7.18 (m, 2H) 7.81-7.87 (m, 2H) 8.09 (s, 1H) 8.20 (dd, 1H) 8.37 (d, 1H) 8.80 (s, 1H) 8.83 (d, 1H).

### Example H9: Preparation of 1-[5-ethylsulfonyl-6-[2-oxo-1-(2,2,2-trifluoroethyl)-1,7-naphthyridin-6-yl]-3-pyridylicyclopropanecarbonitrile (compound P10)

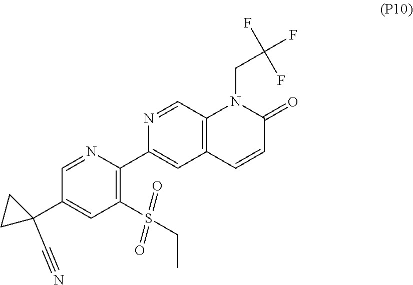 [SMILES: CCS(=O)(=O)c1cc(C2(C#N)CC2)cnc1-c1cc2ccc(=O)n(CC(F)(F)F)c2cn1]

### Step 1: Preparation of 6-bromo-1-(2,2,2-trifluoroethyl)-1,7-naphthyridin-2-one (intermediate I-28)

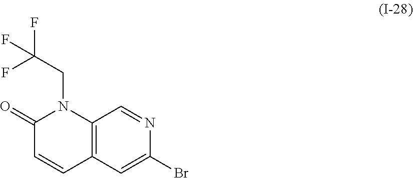 [SMILES: O=c1ccc2cc(Br)ncc2n1CC(F)(F)F]

To a solution of 6-bromo-1H-1,7-naphthyridin-2-one (intermediate I-8 prepared as described above, 6.00 g, 25.1 mmol) in tetrahydrofuran (18 ml) were added potassium carbonate (12.12 g, 87.7 mmol), followed by 2,2,2-trifluoroethyl trifluoromethanesulfonate (18.0 g, 75.19 mmol). The reaction mixture was heated to 75° C. for 5 hours, then the solvent removed under reduced pressure. Ice cold water was added to the residue and the product extracted with ethyl acetate (2×150 ml). The combined organic layers were dried over anhydrous sodium sulfate, filtered and concentrated under reduced pressure. Purification of the crude material by flash chromatography over silica gel (gradient ethyl acetate in cyclohexane) afforded the desired product as a colorless solid (5.0 g). ¹H NMR (400 MHz, CDCl₃) δ ppm 5.00 (br d, 2H) 6.95 (d, 1H) 7.62-7.68 (m, 2H) 8.58 (s, 1H).

### Step 2: Preparation of 1-[5-fluoro-1-oxido-6-[2-oxo-1-(2,2,2-trifluoroethyl)-1,7-naphthyridin-6-yl]pyridin-1-ium-3-yl]cyclopropanecarbonitrile (intermediate I-29)

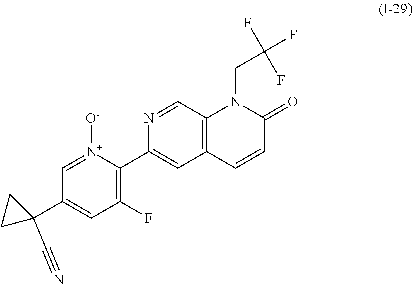 [SMILES: N#CC1(c2cc(F)c(-c3cc4ccc(=O)n(CC(F)(F)F)c4cn3)[n+]([O-])c2)CC1]

A solution of 1-(5-fluoro-1-oxido-pyridin-1-ium-3-yl)cyclopropanecarbonitrile (prepared as described in WO 20/182577, 2.61 g, 14.66 mmol) in tetrahydrofuran (30 ml) was degassed for 10 minutes and cooled to 10° C. 2,2,6,6-Tetramethylpiperidinylzinc chloride lithium chloride complex (1.0 mol/L in THF, 14.66 ml, 14.66 mmol) was added dropwise at 10° C. and stirring continued for an additional 15 minutes at 10° C. A solution of 6-bromo-1-(2,2,3,3,3-pentafluoropropyl)-1,7-naphthyridin-2-one (intermediate I-28 prepared as described above, 3.0 g, 9.77 mmol) in tetrahydrofuran (30 ml) was prepared in a separate flask and degassed with nitrogen for 10 minutes. Both solutions were mixed at 10° C., then [1,1′-bis(diphenylphosphino)ferrocene]dichloropalladium(II) (474 mg, 0.64 mmol) was added and the reaction mixture heated to 60° C. for 15 hours. Aqueous saturated sodium bicarbonate (60 ml) was added and the product extracted with ethyl acetate (3×60 ml). The combined organic layers were dried over anhydrous sodium sulfate, filtered and concentrated under reduced pressure. Purification of the crude material by flash chromatography over silica gel (0-10% methanol in ethyl acetate) afforded the desired product (2.47 g). LCMS (method 1): 405 (M+H)⁺, Rt 0.94 min.

### Step 3: Preparation of 1-[5-ethylsulfanyl-1-oxido-6-[2-oxo-1-(2,2,2-trifluoroethyl)-1,7-naphthyridin-6-yl]pyridin-1-ium-3-yl]cyclopropanecarbonitrile (intermediate I-30)

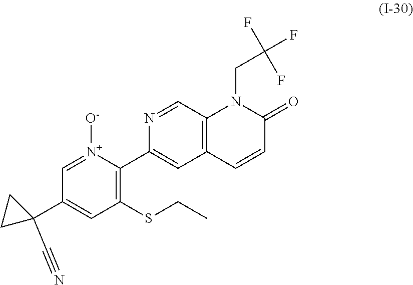 [SMILES: CCSc1cc(C2(C#N)CC2)c[n+]([O-])c1-c1cc2ccc(=O)n(CC(F)(F)F)c2cn1]

To a solution of 1-[5-fluoro-1-oxido-6-[2-oxo-1-(2,2,2-trifluoroethyl)-1,7-naphthyridin-6-yl]pyridin-1-ium-3-yl]cyclopropanecarbonitrile (intermediate I-29 prepared as described above, 2.47 g, 6.11 mmol) in N,N-dimethylformamide (15 ml) was added sodium ethanethiolate (771 mg. 9.16 mmol). The reaction mixture was stirred at room temperature for 2 hours, then ice cold water (100 ml) was added and the product extracted three times with ethyl acetate (3×100 ml). The combined organic layers were washed with water and brine, dried over anhydrous sodium sulfate, filtered and concentrated under reduced pressure. Purification of the crude material by flash chromatography over silica gel (0 to 10% methanol in ethyl acetate) afforded the desired product as a solid (1.77 g), mp: 231-232° C. LCMS (method 1): 447 (M+H)⁺, Rt 0.99 min.

### Step 4: Preparation of 1-[5-ethylsulfanyl-6-[2-oxo-1-(2,2,2-trifluoroethyl)-1,7-naphthyridin-6-yl]-3-pyridylicyclopropanecarbonitrile (compound P15)

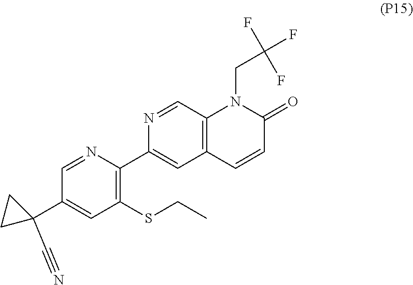 [SMILES: CCSc1cc(C2(C#N)CC2)cnc1-c1cc2ccc(=O)n(CC(F)(F)F)c2cn1]

To a solution of 1-[5-ethylsulfanyl-1-oxido-6-[2-oxo-1-(2,2,2-trifluoroethyl)-1,7-naphthyridin-6-yl]pyridin-1-ium-3-yl]cyclopropanecarbonitrile (intermediate I-30 prepared as described above, 1.77 g, 3.96 mmol) in dry acetonitrile (35.4 ml) was added 4,4,5,5-tetramethyl-2-(4,4,5,5-tetramethyl-1,3,2-dioxaborolan-2-yl)-1,3,2-dioxaborolane (1.51 g, 5.94 mmol). The mixture was stirred 3 hours at 70° C. and 12 hours at 60° C. After dilution with water (100 ml), the product was extracted with ethyl acetate (3×100 ml), the combined organic layers washed with brine (50 ml), dried over anhydrous sodium sulfate and concentrated under reduced pressure. Purification of the crude material by flash chromatography over silica gel (gradient 0 to 40% ethyl acetate in cyclohexane) afforded the desired product as an off white solid (1.09 g). LCMS (method 1): 431 (M+H)⁺, Rt 1.10 min. ¹H NMR (400 MHz, CDCl₃) δ ppm 1.38 (t, 3H) 1.52-1.58 (m, 2H) 1.85-1.90 (m, 2H) 2.98 (q, 2H) 5.08 (br d, 2H) 6.95 (d, 1H) 7.71 (d, 1H) 7.81 (d, 1H) 8.24-8.30 (m, 2H) 8.94 (s, 1H).

### Step 5: Preparation of 1-[5-ethylsulfonyl-6-[2-oxo-1-(2,2,2-trifluoroethyl)-1,7-naphthyridin-6-yl]-3-pyridylicyclopropanecarbonitrile (compound P10)

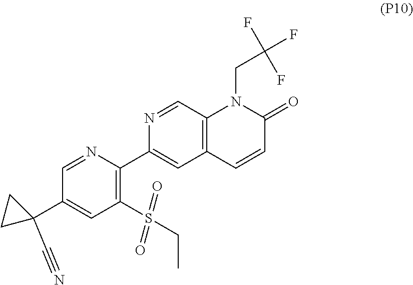 [SMILES: CCS(=O)(=O)c1cc(C2(C#N)CC2)cnc1-c1cc2ccc(=O)n(CC(F)(F)F)c2cn1]

To a solution of 1-[5-ethylsulfanyl-6-[2-oxo-1-(2,2,2-trifluoroethyl)-1,7-naphthyridin-6-yl]-3-pyridyl]cyclopropanecarbonitrile (compound P15 prepared as described above, 350 mg, 0.813 mmol) in acetonitrile (7 ml) at 0° C. was added 3-chlorobenzenecarboperoxoic acid (70%, 441 mg, 1.79 mmol). The mixture was stirred for 2 hours at room temperature, then ice cold water (20 ml) was added and the solution basified with aqueous 2N sodium hydroxide (20 ml). The product was extracted with ethyl acetate (3×20 ml), the combined organic layers washed brine (20 ml), dried over anhydrous sodium sulfate, filtered and concentrated under reduced pressure. Purification of the crude material by flash chromatography over silica gel (gradient 0 to 80% ethyl acetate in cyclohexane) afforded the desired product as a colorless solid (294 mg), mp: 225-226° C. LCMS (method 1): 463 (M+H)⁺, Rt 1.03 min.

¹H NMR (400 MHz, CDCl₃) δ ppm 1.43 (t, 3H) 1.60-1.65 (m, 2H) 1.95-2.00 (m, 2H) 3.99 (q, 2H) 5.09 (br d, 2H) 6.98 (d, 1H) 7.82 (d, 1H) 8.09 (s, 1H) 8.25 (d, 1H) 8.81 (s, 1H) 8.97 (d, 1H).

### Example H10: Preparation of 6-[3-ethylsulfonyl-6-(triazol-2-yl)-2-pyridyl]-1-(2,2,3,3,3-pentafluoropropyl)-1,7-naphthyridin-2-one (compound P11) and 6-[3-ethylsulfonyl-6-(triazol-1-yl)-2-pyridyl]-1-(2,2,3,3,3-pentafluoropropyl)-1,7-naphthyridin-2-one (compound P12)

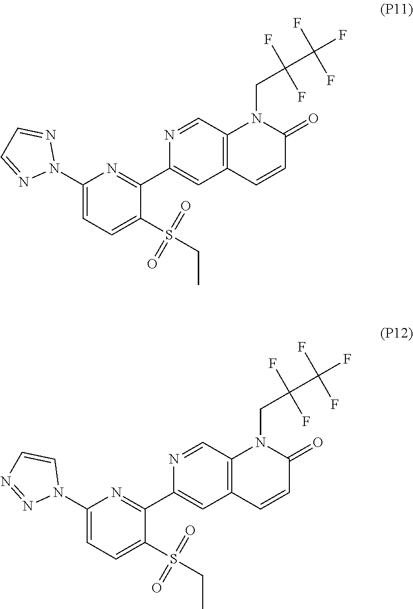 [SMILES: CCS(=O)(=O)c1ccc(-n2nccn2)nc1-c1cc2ccc(=O)n(CC(F)(F)C(F)(F)F)c2cn1 ; CCS(=O)(=O)c1ccc(-n2ccnn2)nc1-c1cc2ccc(=O)n(CC(F)(F)C(F)(F)F)c2cn1]

To a solution of 6-(6-chloro-3-ethylsulfonyl-2-pyridyl)-1-(2,2,3,3,3-pentafluoropropyl)-1,7-naphthyridin-2-one (intermediate I-15 prepared as described above, 400 mg, 0.83 mmol) in acetonitrile (4 ml) were added 2H-triazole (86 mg, 1 0.25 mmol) and potassium carbonate (172 mg, 1 0.25 mmol). The reaction mixture was stirred at 9000 for 1 hour, then diluted with water (50 ml) and the precipitated product was filtered off. Purification of the crude material by reversed phase column chromatography allowed to separate both isomers.

Compound P11: LCMS (method 1): 515 (M+H)⁺, Rt 1.08 min.

¹H NMR (400 MHz, DMSO-d₆) δ ppm 1.32 (t, J=7.40 Hz, 3H) 4.05 (q, J=7.40 Hz, 2H) 5.47 (m, 2H) 7.08 (d, J=9.6 Hz, 1H) 8.12 (d, J=1.35 Hz, 1H) 8.21 (d, J=9.6 Hz, 1H) 8.45 (d, J=8.56 Hz, 1H) 8.48 (s, 1H) 8.75 (d, J=8.56 Hz, 1H) 9.12 (d, J=1.34 Hz, 1H) 9.17 (s, 1H).

Compound P12: LCMS (method 1): 515 (M+H)+, Rt 1.07 min.

¹H NMR (400 MHz, DMSO-d₆) δ ppm 1.30 (t, J=7.40 Hz, 3H) 3.99 (q, J=7.40 Hz, 2H) 5.47 (m, 2H) 7.07 (d, J=9.7 Hz, 1H) 8.29 (d, J=9.7 Hz, 1H) 8.30 (s, 1H) 8.34 (br s, 2H), 8.36 (d, J=8.68 Hz, 1H) 8.70 (d, J=8.68 Hz, 1H) 9.16 (s, 1H).

TABLE P
Examples of compounds of formula (I)

| | | | LCMS | | | |
|---|---|---|---|---|---|---|
| | | | | [M + H]⁺ | | Mp |
| No. | IUPAC name | Structures | Rₜ (min) | (measured) | Method | (° C.) |
| P1 | 6-(3-ethylsulfanyl-2-pyridyl)-1- (2,2,3,3,3-pentafluoropropyl)- 1,7-naphthyridin-2-one | 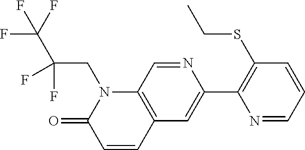 [SMILES: CCSc1cccnc1-c1cc2ccc(=O)n(CC(F)(F)C(F)(F)F)c2cn1] | 1.07 | 416 | 1 | — |
| P2 | 6-(3-ethylsulfonyl-2-pyridyl)-1- (2,2,3,3,3-pentafluoropropyl)- 1,7-naphthyridin-2-one | 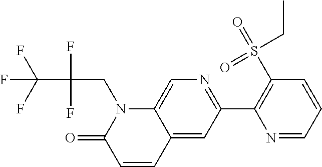 [SMILES: CCS(=O)(=O)c1cccnc1-c1cc2ccc(=O)n(CC(F)(F)C(F)(F)F)c2cn1] | 0.97 | 448 | 1 | 217- 219 |
| P3 | 1-[5-ethylsulfonyl-6-[2-oxo-1- (2,2,3,3,3-pentafluoropropyl)- 1,7-naphthyridin-6-yl]-3- pyridyl]cyclopropanecarbonitrile | 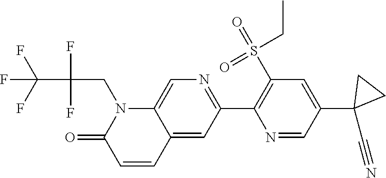 [SMILES: CCS(=O)(=O)c1cc(C2(C#N)CC2)cnc1-c1cc2ccc(=O)n(CC(F)(F)C(F)(F)F)c2cn1] | 1.07 | 513 | 1 | 224- 226 |
| P4 | 6-[3-ethylsulfonyl-6-(1,2,4- triazol-1-yl)-2-pyridyl]-1- (2,2,3,3,3-pentafluoropropyl)- 1,7-naphthyridin-2-one | 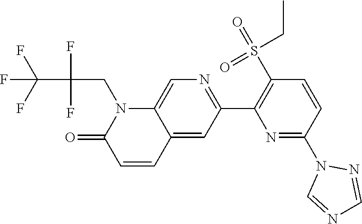 [SMILES: CCS(=O)(=O)c1ccc(-n2cncn2)nc1-c1cc2ccc(=O)n(CC(F)(F)C(F)(F)F)c2cn1] | 1.07 | 515 | 1 | 314- 316 |
| P5 | 6-(6-cyclopropyl-3- ethylsulfonyl-2-pyridyl)-1- (2,2,3,3,3-pentafluoropropyl)- 1,7-naphthyridin-2-one | 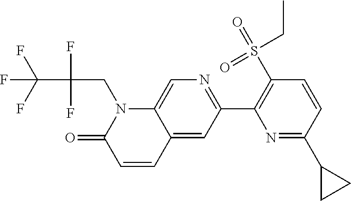 [SMILES: CCS(=O)(=O)c1ccc(C2CC2)nc1-c1cc2ccc(=O)n(CC(F)(F)C(F)(F)F)c2cn1] | 1.16 | 488 | 1 | 212- 214 |
| P6 | 2-[5-ethylsulfonyl-6-[2-oxo-1- (2,2,3,3,3-pentafluoropropyl)- 1,7-naphthyridin-6-yl]-3- pyridyl]-2-methyl-propanenitrile | 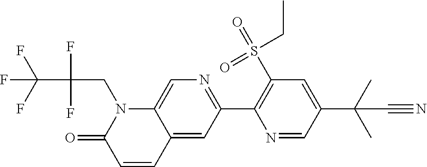 [SMILES: CCS(=O)(=O)c1cc(C(C)(C)C#N)cnc1-c1cc2ccc(=O)n(CC(F)(F)C(F)(F)F)c2cn1] | 1.02 | 515 | 1 | 245- 247 |
| P7 | N-[5-ethylsulfonyl-6-[2-oxo-1- (2,2,3,3,3-pentafluoropropyl)- 1,7-naphthyridin-6-yl]-3- pyridyl]-N-methyl-acetamide | 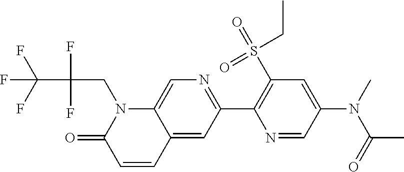 [SMILES: CCS(=O)(=O)c1cc(N(C)C(C)=O)cnc1-c1cc2ccc(=O)n(CC(F)(F)C(F)(F)F)c2cn1] | 0.99 | 519 | 1 | 280- 282 |
| P8 | 6-[5-(3-chloropyrazol-1-yl)-3- ethylsulfonyl-2-pyridyl]-1- (2,2,3,3,3-pentafluoropropyl)- 1,7-naphthyridin-2-one | 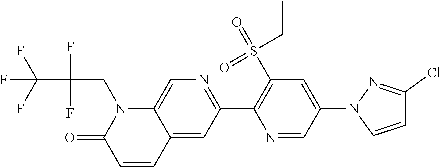 [SMILES: CCS(=O)(=O)c1cc(-n2ccc(Cl)n2)cnc1-c1cc2ccc(=O)n(CC(F)(F)C(F)(F)F)c2cn1] | 1.13 | 548/550 | 1 | 277- 278 |
| P9 | 6-[3-ethylsulfonyl-5-(2- pyridyloxy)-2-pyridyl]-1- (2,2,3,3,3-pentafluoropropyl)- 1,7-naphthyridin-2-one | 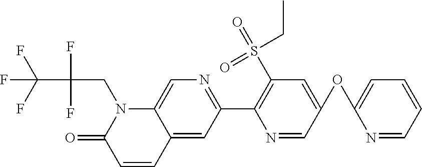 [SMILES: CCS(=O)(=O)c1cc(Oc2ccccn2)cnc1-c1cc2ccc(=O)n(CC(F)(F)C(F)(F)F)c2cn1] | 1.11 | 541 | 1 | 216- 218 |
| P10 | 1-[5-ethylsulfonyl-6-[2-oxo-1- (2,2,2-trifluoroethyl)-1,7- naphthyridin-6-yl]-3- pyridyl]cyclopropanecarbonitrile | 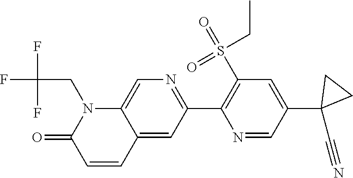 [SMILES: CCS(=O)(=O)c1cc(C2(C#N)CC2)cnc1-c1cc2ccc(=O)n(CC(F)(F)F)c2cn1] | 1.03 | 463 | 1 | 225- 226 |
| P11 | 6-[3-ethylsulfonyl-6-(triazol-2- yl)-2-pyridyl]-1-(2,2,3,3,3- pentafluoropropyl)-1,7- naphthyridin-2-one | 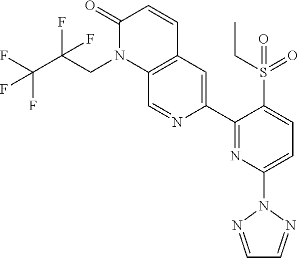 [SMILES: CCS(=O)(=O)c1ccc(-n2nccn2)nc1-c1cc2ccc(=O)n(CC(F)(F)C(F)(F)F)c2cn1] | 1.08 | 515 | 1 | 301- 302 |
| P12 | 6-[3-ethylsulfonyl-6-(triazol-1- yl)-2-pyridyl]-1-(2,2,3,3,3- pentafluoropropyl)-1,7- naphthyridin-2-one | 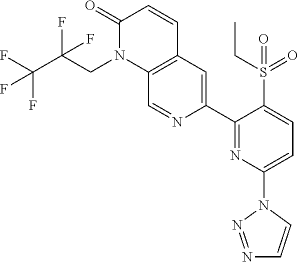 [SMILES: CCS(=O)(=O)c1ccc(-n2ccnn2)nc1-c1cc2ccc(=O)n(CC(F)(F)C(F)(F)F)c2cn1] | 1.07 | 515 | 1 | 294- 296 |
| P13 | 1-[5-ethylsulfanyl-6-[2-oxo-1- (2,2,3,3,3-pentafluoropropyl)- 1,7-naphthyridin-6-yl]-3- pyridyl]cyclopropanecarbonitrile | 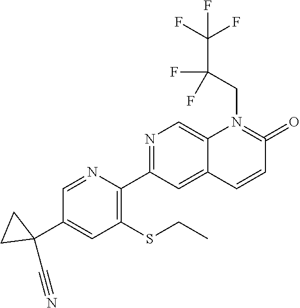 | 1.12 | 481 | 1 | 188- 190 |
| P14 | 6-[5-(3-chloropyrazol-1-yl)-3- ethylsulfanyl-2-pyridyl]-1- (2,2,3,3,3-pentafluoropropyl)- 1,7-naphthyridin-2-one | 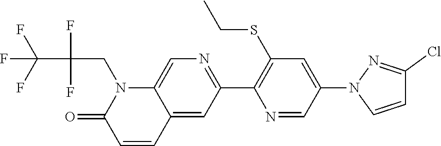 [SMILES: CCSc1cc(-n2ccc(Cl)n2)cnc1-c1cc2ccc(=O)n(CC(F)(F)C(F)(F)F)c2cn1] | 1.20 | 516/518 | 1 | 231- 232 |
| P15 | 1-[5-ethylsulfanyl-6-[2-oxo-1- (2,2,2-trifluoroethyl)-1,7- naphthyridin-6-yl]-3- pyridyl]cyclopropanecarbonitrile | 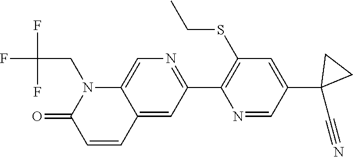 [SMILES: CCSc1cc(C2(C#N)CC2)cnc1-c1cc2ccc(=O)n(CC(F)(F)F)c2cn1] | 1.10 | 431 | 1 | 208- 209 |
| P16 | 6-[3-ethylsulfanyl-5-(2- pyridyloxy)-2-pyridyl]-1- (2,2,3,3,3-pentafluoropropyl)- 1,7-naphthyridin-2-one | 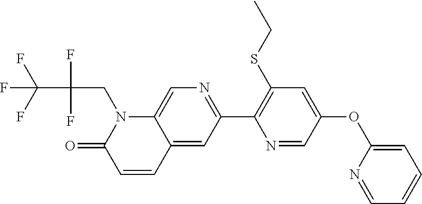 [SMILES: CCSc1cc(Oc2ccccn2)cnc1-c1cc2ccc(=O)n(CC(F)(F)C(F)(F)F)c2cn1] | 1.17 | 509 | 1 | — |
| P17 | 2-[5-ethylsulfanyl-6-[2-oxo-1- (2,2,3,3,3-pentafluoropropyl)- 1,7-naphthyridin-6-yl]-3- pyridyl]-2-methyl-propanenitrile | 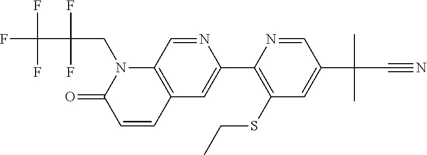 [SMILES: CCSc1cc(C(C)(C)C#N)cnc1-c1cc2ccc(=O)n(CC(F)(F)C(F)(F)F)c2cn1] | 1.11 | 484 | 1 | — |

TABLE 1
Examples of intermediates

| | | | LCMS | | | |
|---|---|---|---|---|---|---|
| | | | | [M + H]⁺ | | Mp |
| No. | IUPAC name | Structures | Rₜ (min) | (measured) | Method | (° C.) |
| I-1 | ethyl 3-(2-chloro-5-nitro-4- pyridyl)-2-oxo-propanoate | 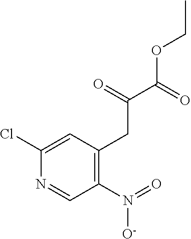 | 0.99 | 273 | 1 | — |
| I-2 | ethyl 3-(2-chloro-5-nitro-4- pyridyl)-2-hydroxy-propanoate | 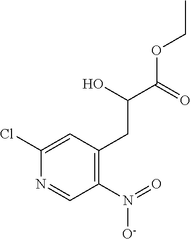 | 0.89 | 275 | 1 | — |
| I-3 | 6-chloro-1H-1,7-naphthyridin- 2-one | 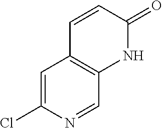 [SMILES: O=c1ccc2cc(Cl)ncc2[nH]1] | 0.38 | 181 | 1 | — |
| I-4 | 6-chloro-1-(2,2,3,3,3- pentafluoropropyl)-1,7- naphthyridin-2-one | 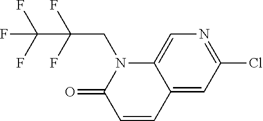 [SMILES: O=c1ccc2cc(Cl)ncc2n1CC(F)(F)C(F)(F)F] | 1.03 | 313 | 1 | — |
| I-5 | 6-(3-fluoro-2-pyridyl)-1- (2,2,3,3,3-pentafluoropropyl)- 1,7-naphthyridin-2-one | 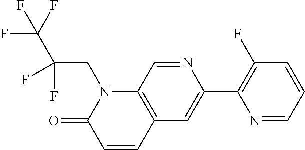 [SMILES: O=c1ccc2cc(-c3ncccc3F)ncc2n1CC(F)(F)C(F)(F)F] | 0.97 | 374 | 1 | — |
| I-6 | (E)-3-(2-bromo-5-nitro-4- pyridyl)-2-hydroxy-prop-2- enoate | 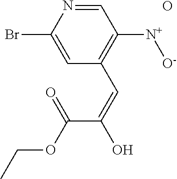 | 0.91 | 315/317 [M − H]− | 1 | — |
| I-7 | (E)-3-(5-amino-2-bromo-4- pyridyl)prop-2-enoic acid | 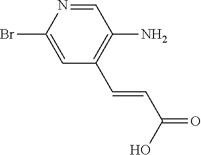 [SMILES: Nc1cnc(Br)cc1/C=C/C(=O)O] | 0.20 | 243/245 | 1 | — |
| I-8 | 6-bromo-1H-1,7-naphthyridin- 2-one | 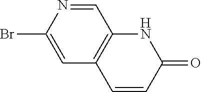 [SMILES: O=c1ccc2cc(Br)ncc2[nH]1] | 0.82 | 225/227 | 1 | — |
| I-9 | 6-bromo-1-(2,2,3,3,3- pentafluoropropyl)-1,7- naphthyridin-2-one | 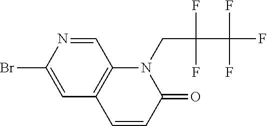 [SMILES: O=c1ccc2cc(Br)ncc2n1CC(F)(F)C(F)(F)F] | 1.00 | 357/359 | 1 | — |
| I-10 | 1-[5-fluoro-1-oxido-6-[2-oxo-1- (2,2,3,3,3-pentafluoropropyl)- 1,7-naphthyridin-6-yl]pyridin-1- ium-3- yl]cyclopropanecarbonitrile | 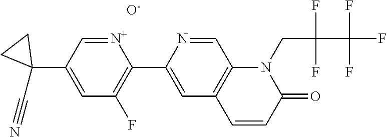 | 0.99 | 455 | 1 | — |
| I-11 | 1-[5-ethylsulfanyl-1-oxido-6-[2- oxo-1-(2,2,3,3,3- pentafluoropropyl)-1,7- naphthyridin-6-yl]pyridin-1-ium- 3-yl]cyclopropanecarbonitrile | 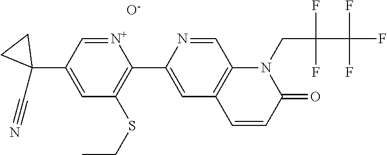 | 1.01 | 497 | 1 | — |
| I-12 | 6-(3-fluoro-1-oxido-pyridin-1- ium-2-yl)-1-(2,2,3,3,3- pentafluoropropyl)-1,7- naphthyridin-2-one | 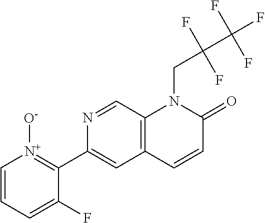 [SMILES: O=c1ccc2cc(-c3c(F)ccc[n+]3[O-])ncc2n1CC(F)(F)C(F)(F)F] | 0.94 | 390 | 1 | — |
| I-13 | 6-(3-ethylsulfanyl-1-oxido- pyridin-1-ium-2-yl)-1- (2,2,3,3,3-pentafluoropropyl)- 1,7-naphthyridin-2-one | 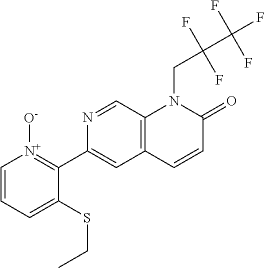 [SMILES: CCSc1ccc[n+]([O-])c1-c1cc2ccc(=O)n(CC(F)(F)C(F)(F)F)c2cn1] | 0.77 | 432 | 1 | — |
| I-14 | 6-(6-chloro-3-ethylsulfanyl-2- pyridyl)-1-(2,2,3,3,3- pentafluoropropyl)-1,7- naphthyridin-2-one | 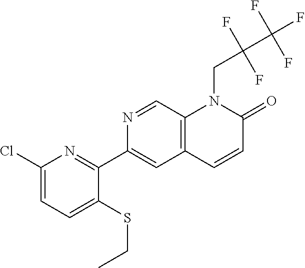 [SMILES: CCSc1ccc(Cl)nc1-c1cc2ccc(=O)n(CC(F)(F)C(F)(F)F)c2cn1] | 1.18 | 451 | 1 | — |
| I-15 | 6-(6-chloro-3-ethylsulfonyl-2- pyridyl)-1-(2,2,3,3,3- pentafluoropropyl)-1,7- naphthyridin-2-one | 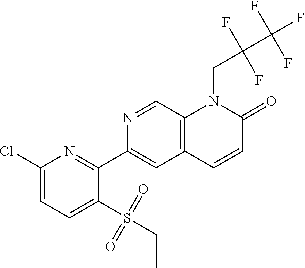 [SMILES: CCS(=O)(=O)c1ccc(Cl)nc1-c1cc2ccc(=O)n(CC(F)(F)C(F)(F)F)c2cn1] | 1.10 | 483 | 1 | — |
| I-16 | 2-[5-fluoro-1-oxido-6-[2-oxo-1- (2,2,3,3,3-pentafluoropropyl)- 1,7-naphthyridin-6-yl]pyridin-1- ium-3-yl]-2-methyl- propanenitrile | 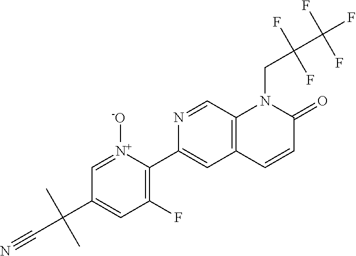 | 0.90 | 457 | 1 | — |
| I-17 | 2-[5-ethylsulfanyl-1-oxido-6-[2- oxo-1-(2,2,3,3,3- pentafluoropropyl)-1,7- naphthyridin-6-yl]pyridin-1-ium- 3-yl]-2-methyl-propanenitrile | 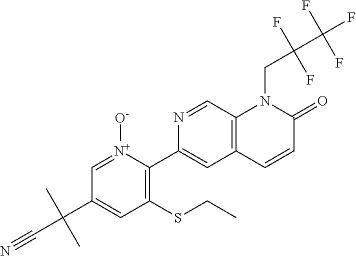 [SMILES: CCSc1cc(C(C)(C)C#N)c[n+]([O-])c1-c1cc2ccc(=O)n(CC(F)(F)C(F)(F)F)c2cn1] | 0.93 | 499 | 1 | — |
| I-18 | tert-butyl N-[5-fluoro-1-oxido-6- [2-oxo-1-(2,2,3,3,3- pentafluoropropyl)-1,7- naphthyridin-6-yl]pyridin-1-ium- 3-yl]-N-methyl-carbamate | 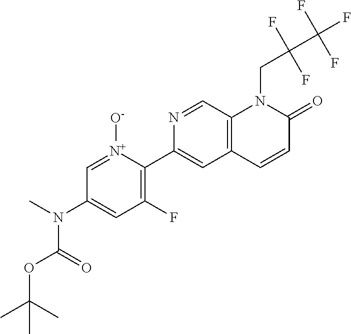 [SMILES: CN(C(=O)OC(C)(C)C)c1cc(F)c(-c2cc3ccc(=O)n(CC(F)(F)C(F)(F)F)c3cn2)[n+]([O-])c1] | 1.04 | 519 | 1 | — |
| I-19 | tert-butyl N-[5-ethylsulfanyl-1- oxido-6-[2-oxo-1-(2,2,3,3,3- pentafluoropropyl)-1,7- naphthyridin-6-yl]pyridin-1-ium- 3-yl]-N-methyl-carbamate | 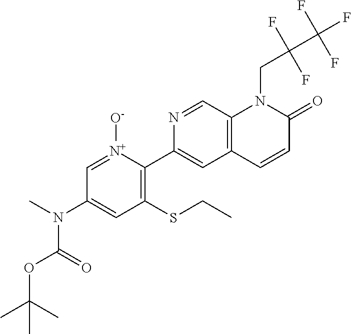 [SMILES: CCSc1cc(N(C)C(=O)OC(C)(C)C)c[n+]([O-])c1-c1cc2ccc(=O)n(CC(F)(F)C(F)(F)F)c2cn1] | 1.10 | 561 | 1 | — |
| I-20 | tert-butyl N-[5-ethylsulfanyl-6- [2-oxo-1-(2,2,3,3,3- pentafluoropropyl)-1,7- naphthyridin-6-yl]-3-pyridyl]-N- methyl-carbamate | 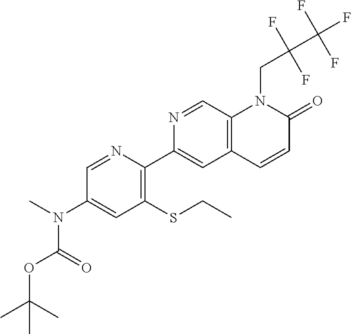 [SMILES: CCSc1cc(N(C)C(=O)OC(C)(C)C)cnc1-c1cc2ccc(=O)n(CC(F)(F)C(F)(F)F)c2cn1] | 1.24 | 545 | 1 | 172- 174 |
| I-21 | tert-butyl N-[5-ethylsulfonyl-6- [2-oxo-1-(2,2,3,3,3- pentafluoropropyl)-1,7- naphthyridin-6-yl]-3-pyridyl]-N- methyl-carbamate | 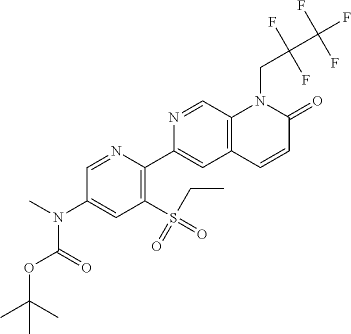 [SMILES: CCS(=O)(=O)c1cc(N(C)C(=O)OC(C)(C)C)cnc1-c1cc2ccc(=O)n(CC(F)(F)C(F)(F)F)c2cn1] | 1.13 | 577 | 1 | 196- 198 |
| I-22 | 6-[3-ethylsulfonyl-5- (methylamino)-2-pyridyl]-1- (2,2,3,3,3-pentafluoropropyl)- 1,7-naphthyridin-2-one | 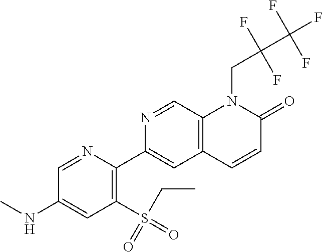 [SMILES: CCS(=O)(=O)c1cc(NC)cnc1-c1cc2ccc(=O)n(CC(F)(F)C(F)(F)F)c2cn1] | 1.04 | 477 | 1 | 224- 226 |
| I-23 | (3-chloropyrazol-1-yl)-5-fluoro- 1-oxido-pyridin-1-ium | 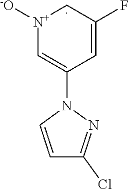 | 0.58 | 214/216 | 1 | — |
| I-24 | 6-[5-(3-chloropyrazol-1-yl)-3- fluoro-1-oxido-pyridin-1-ium-2- yl]-1-(2,2,3,3,3- pentafluoropropyl)-1,7- naphthyridin-2-one | 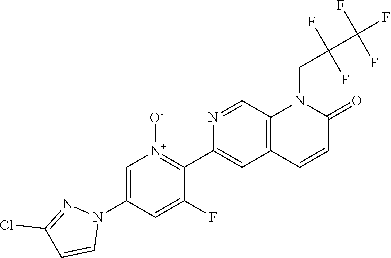 [SMILES: O=c1ccc2cc(-c3c(F)cc(-n4ccc(Cl)n4)c[n+]3[O-])ncc2n1CC(F)(F)C(F)(F)F] | 1.07 | 490/492 | 1 | — |
| I-25 | 6-[5-(3-chloropyrazol-1-yl)-3- fluoro-2-pyridyl]-1 -(2,2,3,3,3- pentafluoropropyl)-1,7- naphthyridin-2-one | 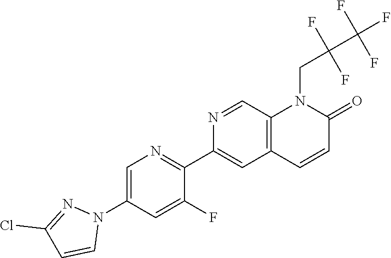 [SMILES: O=c1ccc2cc(-c3ncc(-n4ccc(Cl)n4)cc3F)ncc2n1CC(F)(F)C(F)(F)F] | 1.11 | 474/476 | 1 | — |
| I-26 | 6-[3-fluoro-1-oxido-5-(2- pyridyloxy)pyridin-1-ium-2-yl]- 1-(2,2,3,3,3- pentafluoropropyl)-1,7- naphthyridin-2-one | 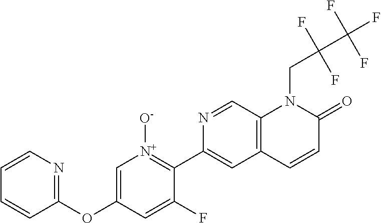 [SMILES: O=c1ccc2cc(-c3c(F)cc(Oc4ccccn4)c[n+]3[O-])ncc2n1CC(F)(F)C(F)(F)F] | 1.00 | 483 | 1 | — |
| I-27 | 6-[3-ethylsulfanyl-1-oxido-5-(2- pyridyloxy)pyridin-1-ium-2-yl]- 1-(2,2,3,3,3- pentafluoropropyl)-1,7- naphthyridin-2-one | 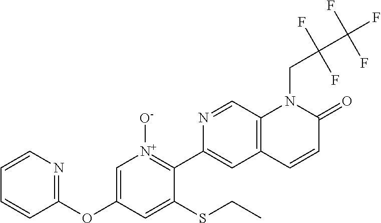 [SMILES: CCSc1cc(Oc2ccccn2)c[n+]([O-])c1-c1cc2ccc(=O)n(CC(F)(F)C(F)(F)F)c2cn1] | 1.03 | 525 | 1 | — |
| I-28 | 6-bromo-1-(2,2,2- trifluoroethyl)-1,7-naphthyridin- 2-one | 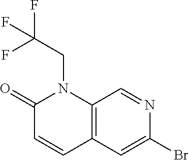 [SMILES: O=c1ccc2cc(Br)ncc2n1CC(F)(F)F] | ¹H NMR (400 MHz, CDCl₃) δ ppm 5.00 (br d, 2 H) 6.95 (d, 1 H) 7.62- 7.68 (m, 2 H) 8.58 (s, 1 H). | | | |
| I-29 | 1-[5-fluoro-1-oxido-6-[2-oxo-1- (2,2,2-trifluoroethyl)-1,7- naphthyridin-6-yl]pyridin-1-ium- 3-yl]cyclopropanecarbonitrile | 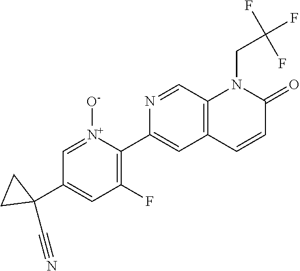 [SMILES: N#CC1(c2cc(F)c(-c3cc4ccc(=O)n(CC(F)(F)F)c4cn3)[n+]([O-])c2)CC1] | 0.94 | 405 | 1 | |
| I-30 | 1-[5-ethylsulfanyl-1-oxido-6-[2- oxo-1-(2,2,2-trifluoroethyl)-1,7- naphthyridin-6-yl]pyridin-1-ium- 3-yl]cyclopropanecarbonitrile | 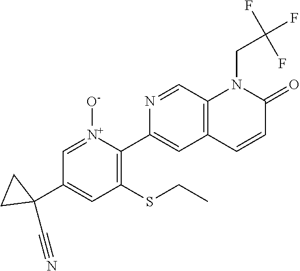 [SMILES: CCSc1cc(C2(C#N)CC2)c[n+]([O-])c1-c1cc2ccc(=O)n(CC(F)(F)F)c2cn1] | 0.99 | 447 | 1 | 231- 232 |

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use. Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula I with active ingredients are preferred (the abbreviation “TX” means “one compound selected from the group consisting of the compounds described in Tables A-1 to A-60 and Tables B-1 to B-60, and Table P of the present invention”): an adjuvant selected from the group of substances consisting of petroleum oils (alternative name) (628)+TX;

an insect control active substance selected from Abamectin+TX, Acequinocyl+TX, Acetamiprid+TX, Acetoprole+TX, Acrinathrin+TX, Acynonapyr+TX, Afidopyropen+TX, Afoxolaner+TX, Alanycarb+TX, Allethrin+TX, Alpha-Cypermethrin+TX, Alphamethrin+TX, Amidoflumet+TX, Aminocarb+TX, Azocyclotin+TX, Bensultap+TX, Benzoximate+TX, Benzpyrimoxan+TX, Betacyfluthrin+TX, Beta-cypermethrin+TX, Bifenazate+TX, Bifenthrin+TX, Binapacryl+TX, Bioallethrin+TX, Bioallethrin S)-cyclopentylisomer+TX, Bioresmethrin+TX, Bistrifluron+TX, Broflanilide+TX, Brofluthrinate+TX, Bromophos-ethyl+TX, Buprofezine+TX, Butocarboxim+TX, Cadusafos+TX, Carbaryl+TX, Carbosulfan+TX, Cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2246757-58-2 (or 2249718-27-0)+TX, CAS number: 907187-07-9+TX, Chlorantraniliprole+TX, Chlordane+TX, Chlorfenapyr+TX, Chloroprallethrin+TX, Chromafenozide+TX, Clenpirin+TX, Cloethocarb+TX, Clothianidin+TX, 2-chlorophenyl N-methylcarbamate (CPMC)+TX, Cyanofenphos+TX, Cyantraniliprole+TX, Cyclaniliprole+TX, Cyclobutrifluram+TX, Cycloprothrin+TX, Cycloxaprid+TX, Cycloxaprid+TX, Cyenopyrafen+TX, Cyetpyrafen+TX, Cyflumetofen+TX, Cyfluthrin+TX, Cyhalodiamide+TX, Cyhalothrin+TX, Cypermethrin+TX, Cyphenothrin+TX, Cyproflanilide+TX, Cyromazine+TX, Deltamethrin+TX, Diafenthiuron+TX, Dialifos+TX, Dibrom+TX, Dicloromezotiaz+TX, Diflovidazine+TX, Diflubenzuron+TX, dimpropyridaz+TX, Dinactin+TX, Dinocap+TX, Dinotefuran+TX, Dioxabenzofos+TX, Emamectin (or Emamectin Benzoate)+TX, Empenthrin+TX, Epsilon-momfluorothrin+TX, Epsilon-metofluthrin+TX, Esfenvalerate+TX, Ethion+TX, Ethiprole+TX, Etofenprox+TX, Etoxazole+TX, Famphur+TX, Fenazaquin+TX, Fenfluthrin+TX, Fenitrothion+TX, Fenobucarb+TX, Fenothiocarb+TX, Fenoxycarb+TX, Fenpropathrin+TX, Fenpyroxymate+TX, Fensulfothion+TX, Fenthion+TX, Fentinacetate+TX, Fenvalerate+TX, Fipronil+TX, Flometoquin+TX, Flonicamid+TX, Fluacrypyrim+TX, Fluazaindolizine+TX, Fluazuron+TX, Flubendiamide+TX, Flubenzimine+TX, Flucitrinate+TX, Flucycloxuron+TX, Flucythrinate+TX, Fluensulfone+TX, Flufenerim+TX, Flufenprox+TX, Flufiprole+TX, Fluhexafon+TX, Flumethrin+TX, Fluopyram+TX, Flupentiofenox+TX, Flupyradifurone+TX, Flupyrimin+TX, Fluralaner+TX, Fluvalinate+TX, Fluxametamide+TX, Fosthiazate+TX, Gamma-Cyhalothrin+TX, Gossyplure™+TX, Guadipyr+TX, Halofenozide+TX, Halofenozide+TX, Halfenprox+TX, Heptafluthrin+TX, Hexythiazox+TX, Hydramethylnon+TX, Imicyafos+TX, Imidacloprid+TX, Imiprothrin+TX, Indoxacarb+TX, Iodomethane+TX, Iprodione+TX, Isocycloseram+TX, Isothioate+TX, Ivermectin+TX, Kappa-bifenthrin+TX, Kappa-tefluthrin+TX, Lambda-Cyhalothrin+TX, Lepimectin+TX, Lufenuron+TX, Metaflumizone+TX, Metaldehyde+TX, Metam+TX, Methomyl+TX, Methoxyfenozide+TX, Metofluthrin+TX, Metolcarb+TX, Mexacarbate+TX, Milbemectin+TX, Momfluorothrin+TX, Niclosamide+TX, Nicofluprole+TX; Nitenpyram+TX, Nithiazine+TX, Omethoate+TX, Oxamyl+TX, Oxazosulfyl+TX, Parathion-ethyl+TX, Permethrin+TX, Phenothrin+TX, Phosphocarb+TX, Piperonylbutoxide+TX, Pirimicarb+TX, Pirimiphos-ethyl+TX, Pirimiphos-methyl+TX, Polyhedrosis virus+TX, Prallethrin+TX, Profenofos+TX, Profenofos+TX, Profluthrin+TX, Propargite+TX, Propetamphos+TX, Propoxur+TX, Prothiophos+TX, Protrifenbute+TX, Pyflubumide+TX, Pymetrozine+TX, Pyraclofos+TX, Pyrafluprole+TX, Pyridaben+TX, Pyridalyl+TX, Pyrifluquinazon+TX, Pyrimidifen+TX, Pyriminostrobin+TX, Pyriprole+TX, Pyriproxyfen+TX, Resmethrin+TX, Sarolaner+TX, Selamectin+TX, Silafluofen+TX, Spinetoram+TX, Spinosad+TX, Spirodiclofen+TX, Spiromesifen+TX, Spiropidion+TX, Spirotetramat+TX, Sulfoxaflor+TX, Tebufenozide+TX, Tebufenpyrad+TX, Tebupirimiphos+TX, Tefluthrin+TX, Temephos+TX, Tetrachlorantraniliprole+TX, Tetradiphon+TX, Tetramethrin+TX, Tetramethylfluthrin+TX, Tetranactin+TX, Tetraniliprole+TX, Theta-cypermethrin+TX, Thiacloprid+TX, Thiamethoxam+TX, Thiocyclam+TX, Thiodicarb+TX, Thiofanox+TX, Thiometon+TX, Thiosultap+TX, Tioxazafen+TX, Tolfenpyrad+TX, Toxaphene+TX, Tralomethrin+TX, Transfluthrin+TX, Triazamate+TX, Triazophos+TX, Trichlorfon+TX, Trichloronate+TX, Trichlorphon+TX, Triflumezopyrim+TX, Tyclopyrazoflor+TX, Zeta-Cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, Azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.;

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;

an anthelmintic selected from the group of substances consisting of abamectin (1)+TX, crufomate (1011)+TX, Cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;

an avicide selected from the group of substances consisting of chloralose (127)+TX, endrin (1122)+TX, fenthion (346)+TX, pyridin-4-amine (IUPAC name) (23) and strychnine (745)+TX; a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;

a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;

a soil sterilant selected from the group of substances consisting of iodomethane (IUPAC name) (542) and methyl bromide (537)+TX;

a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX;

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;

a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, Cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;

a nitrification inhibitor selected from the group of substances consisting of potassium ethylxanthate [CCN] and nitrapyrin (580)+TX;

a plant activator selected from the group of substances consisting of acibenzolar (6)+TX, acibenzolar-S-methyl (6)+TX, probenazole (658) and Reynoutria sachalinensis extract (alternative name) (720)+TX;

a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX; a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX;

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;

a virucide selected from the group of substances consisting of imanin (alternative name) [CCN] and ribavirin (alternative name) [CCN]+TX;

a wound protectant selected from the group of substances consisting of mercuric oxide (512)+TX, octhilinone (590) and thiophanate-methyl (802)+TX;

a biologically active substance selected from 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. Iecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B₁+TX, trimedlure B₂+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, EI 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla)+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, and meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, 2-fluoro-N-(3-methoxyphenyl)-9H-purin-6-amine+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, -sodium fluoroacetate+TX, thallium sulfate+TX, warfarin+TX, -2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole-+TX, epoxiconazole+TX, fenbuconazole+TX, fluquinconazole+TX, flusilazole+TX, flutriafol+TX, furametpyr+TX, hexaconazole+TX, imazalil-+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, -simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, -metalaxyl-+TX, Rmetalaxyl+TX, ofurace+TX, oxadixyl+TX, carbendazim+TX, debacarb+TX, fuberidazole-+TX, thiabendazole+TX, chlozolinate+TX, dichlozoline+TX, myclozoline-+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb-+TX, chloro-tha-lonil+TX, cyflufenamid+TX, cymoxanil+TX, cyclobutrifluram+TX, diclocymet+TX, diclomezine-+TX, dicloran+TX, diethofencarb+TX, dimethomorph-+TX, flumorph+TX, dithianon+TX, ethaboxam+TX, etridiazole+TX, famoxadone+TX, fenamidone+TX, fenoxanil+TX, ferimzone+TX, fluazinam+TX, fluopicolide+TX, flusulfamide+TX, fluxapyroxad+TX, -fenhexamid+TX, fosetyl-aluminium-+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1,3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1,1,3-trimethylindan-4-yl) pyridine-3-carboxamide+TX, α-(1,1-dimethylethyl)-α-[4′-(trifluoromethoxy) [1,1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, N-octyl-N′-[2-(octylamino)ethyl]ethane-1,2-diamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N—[(Z)-methoxyiminomethyl]-4-[5-(trifluoromethyl)-1,2,4-oxadiazol-3-yl]benzamide+TX, N—[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);

microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1 Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural Il®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris py. Poae (Camperico®)+TX, Xenorhabdus bovienii+TX, Xenorhabdus nematophilus;

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®);

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus Iounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-Rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX; and

a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.htm.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from Tables A-1 to A-60 and Tables B-1 to B-60, and Table P with active ingredients described above comprises a compound selected from Tables A-1 to A-60 and Tables B-1 to B-60, and Table P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables A-1 to A-60 and Tables B-1 to B-60, and Table P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables A-1 to A-60 and Tables B-1 to B-60, and Table P and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### Biological Examples

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

### Example B1: Activity Against Diabrotica Balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P2, P3, P5, P6, P7, P8, P9, P10, P11, P12, P13, P15.

### Example B2: Activity Against Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation. The following compounds gave an effect of at least 80% in at least one of the two categories (mortality or growth inhibition) at an application rate of 200 ppm: P2, P3, P4, P5, P6, P8, P13.

### Example B3: Activity Against Myzus Persicae (Green Peach Aphid) Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P2, P3, P5, P7, P10.

### Example B4: Activity Against Myzus persicae (Green Peach Aphid) Systemic Activity

Roots of pea seedlings infested with an aphid population of mixed ages were placed directly into aqueous test solutions prepared from 10′000 DMSO stock solutions. The samples were assessed for mortality 6 days after placing seedlings into test solutions.

The following compounds resulted in at least 80% mortality at a test rate of 24 ppm: P2, P3, P7.

### Example B5: Activity Against Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P2, P3, P4, P5, P6, P7, P8, P9, P10, P11, P12, P13, P15.

### Example B6: Activity Against Chilo suppressalis (Striped Rice Stemborer)

24-well microtiter plates with artificial diet were treated with aqueous test solutions prepared from 10′000 ppm DMSO stock solutions by pipetting. After drying, the plates were infested with L2 larvae (6-8 per well). The samples were assessed for mortality, anti-feeding effect, and growth inhibition in comparison to untreated samples 6 days after infestation. Control of Chilo suppressalis by a test sample is given when at least one of the categories mortality, anti-feedant effect, and growth inhibition is higher than the untreated sample.

The following compounds resulted in at least 80% control at an application rate of 200 ppm: P2, P3, P4, P5, P6, P7, P8, P9, P10, P11, P12, P13, P14, P15.

### Example B7: Activity Against Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P2, P3, P6, P8, P9, P10.

### Example B8: Activity Against Carpocapsa (Cydia) Pomonella (Codling Moth)

Diet cubes coated with paraffin were sprayed with diluted test solutions in an application chamber. After drying off the treated cubes (10 replicates) were infested with 1 L1 larvae. Samples were incubated at 26-27° C. and checked 14 days after infestation for mortality and growth inhibition. For example, the following compounds resulted in at least 80% mortality at an application rate of 12.5 ppm: P3, P4, P6.

### Example B9: Activity Against Frankliniella occidentalis (Western Flower Thrips)

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm: P3.

1. A compound of formula (I) 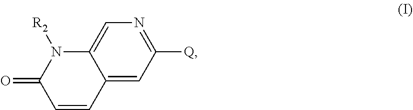
wherein
R₂ is C₁-C₆haloalkyl;
Q is a radical selected from the group consisting of formula Qa and Qb 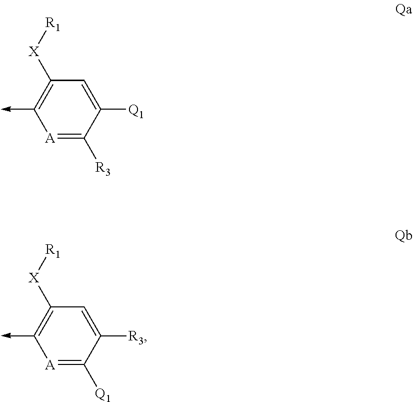
wherein the arrow denotes the point of attachment to the carbon atom of the bicyclic ring;
and wherein A represents CH or N;
X is S, SO, or SO₂;
R₁ is C₁-C₄alkyl or C₃-C₆cycloalkyl-C₁-C₄alkyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system can contain 1, 2 or 3 ring heteroatoms selected from the group consisting of nitrogen, oxygen and sulphur, where said ring system may not contain more than one ring oxygen atom and not more than one ring sulfur atom; or
Q₁ is a five-membered aromatic ring system linked via a ring nitrogen atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl;
and said ring system contains 1, 2 or 3 ring heteroatoms selected from the group consisting of nitrogen, oxygen and sulphur, where said ring system contains at least one ring nitrogen atom and may not contain more than one ring oxygen atom and not more than one ring sulfur atom;
R₃ is hydrogen, halogen or C₁-C₄alkyl;
each R₄ is independently hydrogen, C₁-C₄alkyl or C₃-C₆cycloalkyl; and
R₅ is C₁-C₆alkyl, C₁-C₆haloalkyl or C₃-C₆cycloalkyl;
or an agrochemically acceptable salt, stereoisomer, enantiomer, tautomer or N-oxide of a compound of formula I.

2. A compound of formula I according to claim 1, represented by the compounds of formula (I-1) 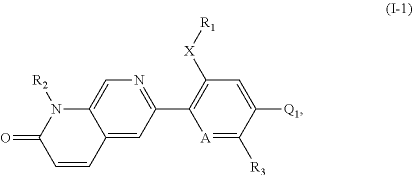
wherein A, X, R₁, and R₂ are as defined under formula I in claim 1, or an agrochemically acceptable salt, stereoisomer, enantiomer, tautomer or N-oxide thereof, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

3. A compound of formula I according to claim 1, represented by the compounds of formula (I-2) 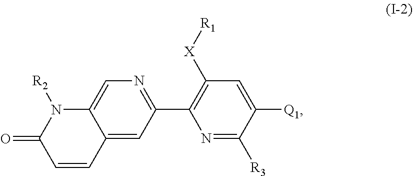
wherein X, R₁ and R₂ are as defined under formula I in claim 1, or an agrochemically acceptable salt, stereoisomer, enantiomer, tautomer or N-oxide thereof, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

4. A compound of formula I according to claim 1, represented by the compounds of formula (I-3) 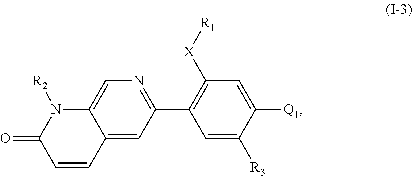
wherein X, R₁ and R₂ are as defined under formula I in claim 1, or an agrochemically acceptable salt, stereoisomer, enantiomer, tautomer or N-oxide thereof, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

5. A compound of formula I according to claim 1, represented by the compounds of formula (I-4) 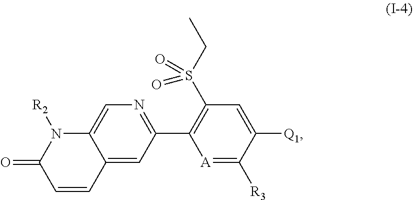
wherein
A is CH or N, preferably N;
R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₄ is independently hydrogen or C₁-C₄alkyl, preferably hydrogen or methyl; and
R₅ is C₁-C₆alkyl or C₃-C₆cycloalkyl, preferably methyl, ethyl or cyclopropyl, more preferably methyl or cyclopropyl.

6. A compound of formula I according to claim 1, represented by the compounds of formula (I-5) 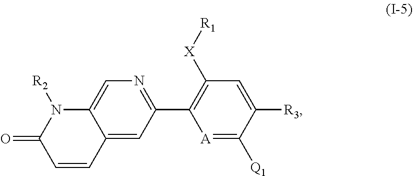
wherein A, X, R₁, and R₂ are as defined under formula I in claim 1, or an agrochemically acceptable salt, stereoisomer, enantiomer, tautomer or N-oxide thereof, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

7. A compound of formula I according to claim 1, represented by the compounds of formula (I-6) 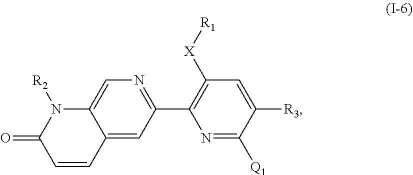
wherein X, R₁ and R₂ are as defined under formula I in claim 1, or an agrochemically acceptable salt, stereoisomer, enantiomer, tautomer or N-oxide thereof, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

8. A compound of formula I according to claim 1, represented by the compounds of formula (I-7) 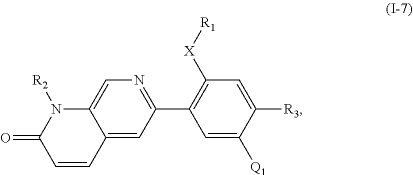
wherein X, R₁ and R₂ are as defined under formula I in claim 1, or an agrochemically acceptable salt, stereoisomer, enantiomer, tautomer or N-oxide thereof, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl.

9. A compound of formula I according to claim 1, represented by the compounds of formula (I-8) 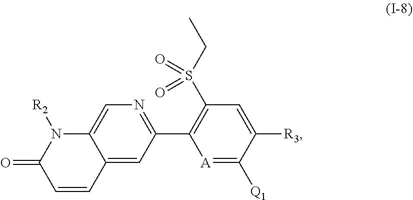
wherein
A is CH or N, preferably N;
R₂ is C₁-C₆haloalkyl, preferably R₂ is C₁-C₆fluoroalkyl, more preferably R₂ is —CH₂CF₂CF₃, —CH₂CF₂CHF₂, —CH₂CF₃, —CH₂CHF₂ or —CH₂CF₂CHFCF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl, preferably hydrogen or methyl; and
R₅ is C₁-C₆alkyl or C₃-C₆cycloalkyl; preferably methyl, ethyl or cyclopropyl, more preferably methyl or cyclopropyl.

10. A compound according tom claim 1, wherein Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl; preferably Q₁ is hydrogen, chlorine, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl, 1,2,4-triazol-1-yl, triazol-1-yl or triazol-2-yl.

11. A compound according to claim 1, wherein R₂ is —CH₂CF₂CF₃, —CH₂CF₂CHF₂, —CH₂CF₃, —CH₂CHF₂ or —CH₂CF₂CHFCF₃; preferably R₂ is —CH₂CF₃, —CH₂CF₂CHF₂ or —CH₂CF₂CF₃; preferably R₂ is —CH₂CF₂CF₃ or —CH₂CF₃.

12. A compound of formula I according to claim 1, selected from the group consisting of:
6-(3-ethylsulfanyl-2-pyridyl)-1-(2,2,3,3,3-pentafluoropropyl)-1,7-naphthyridin-2-one (compound P1); 6-(3-ethylsulfonyl-2-pyridyl)-1-(2,2,3,3,3-pentafluoropropyl)-1,7-naphthyridin-2-one (compound P2); 1-[5-ethylsulfonyl-6-[2-oxo-1-(2,2,3,3,3-pentafluoropropyl)-1,7-naphthyridin-6-yl]-3-pyridyl]cyclopropanecarbonitrile (compound P3); 6-[3-ethylsulfonyl-6-(1,2,4-triazol-1-yl)-2-pyridyl]-1-(2,2,3,3,3-pentafluoropropyl)-1,7-naphthyridin-2-one (compound P4); 6-(6-cyclopropyl-3-ethylsulfonyl-2-pyridyl)-1-(2,2,3,3,3-pentafluoropropyl)-1,7-naphthyridin-2-one (compound P5); 2-[5-ethylsulfonyl-6-[2-oxo-1-(2,2,3,3,3-pentafluoropropyl)-1,7-naphthyridin-6-yl]-3-pyridyl]-2-methyl-propanenitrile (compound P6); N-[5-ethylsulfonyl-6-[2-oxo-1-(2,2,3,3,3-pentafluoropropyl)-1,7-naphthyridin-6-yl]-3-pyridyl]-N-methyl-acetamide (compound P7); 6-[5-(3-chloropyrazol-1-yl)-3-ethylsulfonyl-2-pyridyl]-1-(2,2,3,3,3-pentafluoropropyl)-1,7-naphthyridin-2-one (compound P8); 6-[3-ethylsulfonyl-5-(2-pyridyloxy)-2-pyridyl]-1-(2,2,3,3,3-pentafluoropropyl)-1,7-naphthyridin-2-one (compound P9); 1-[5-ethylsulfonyl-6-[2-oxo-1-(2,2,2-trifluoroethyl)-1,7-naphthyridin-6-yl]-3-pyridyl]cyclopropanecarbonitrile (compound P10); 6-[3-ethylsulfonyl-6-(triazol-2-yl)-2-pyridyl]-1-(2,2,3,3,3-pentafluoropropyl)-1,7-naphthyridin-2-one (compound P11); 6-[3-ethylsulfonyl-6-(triazol-1-yl)-2-pyridyl]-1-(2,2,3,3,3-pentafluoropropyl)-1,7-naphthyridin-2-one (compound P12);1-[5-ethylsulfanyl-6-[2-oxo-1-(2,2,3,3,3-pentafluoropropyl)-1,7-naphthyridin-6-yl]-3-pyridyl]cyclopropanecarbonitrile (compound P13); 6-[5-(3-chloropyrazol-1-yl)-3-ethylsulfanyl-2-pyridyl]-1-(2,2,3,3,3-pentafluoropropyl)-1,7-naphthyridin-2-one (compound P14); 1-[5-ethylsulfanyl-6-[2-oxo-1-(2,2,2-trifluoroethyl)-1,7-naphthyridin-6-yl]-3-pyridyl]cyclopropanecarbonitrile (compound P15); 6-[3-ethylsulfanyl-5-(2-pyridyloxy)-2-pyridyl]-1-(2,2,3,3,3-pentafluoropropyl)-1,7-naphthyridin-2-one (compound P16); and 2-[5-ethylsulfanyl-6-[2-oxo-1-(2,2,3,3,3-pentafluoropropyl)-1,7-naphthyridin-6-yl]-3-pyridyl]-2-methyl-propanenitrile (compound P17).

13. A composition comprising an insecticidally, acaricidally, nematicidally or molluscicidally effective amount of a compound of formula (I), or an agrochemically acceptable salt, stereoisomer, enantiomer, tautomer or N-oxide thereof, as defined in claim 1 and, optionally, an auxiliary or diluent.

14. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in claim 1.

15. A method for the protection of plant propagation material from the attack by insects, acarines, nematodes or molluscs, which comprises treating the propagation material or the site, where the propagation material is planted, with a composition according to claim 13.

16. A compound of formula III 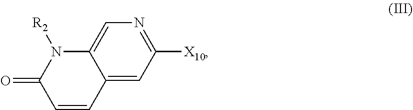
wherein
R₂ are as defined under formula I in claim 1; and
X₁₀ is a halogen or a pseudo-halogen leaving group.

17. A compound of formula X 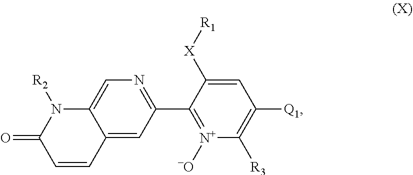
wherein
X is S; and
R₂, Q₁, R₃ and R₁ are as defined under formula I in claim 1.

18. A compound of formula IX 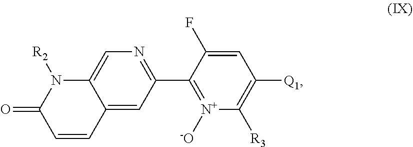
wherein
R₂, Q₁ and R₃ are as defined under formula I in claim 1.

19. A compound of formula V 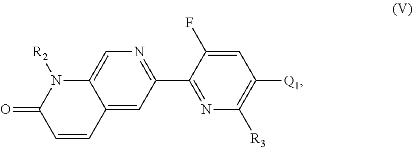
wherein
R₂, Q₁ and R₃ are as defined under formula I in claim 1.

20. A compound of formula XXXIb-1 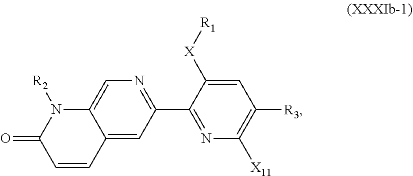
wherein
X is S, SO or SO₂;
X₁₁ is a halogen or a pseudo-halogen leaving group; and
R₁, R₂ and R₃ are as defined under formula I in claim 1.

21. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in a composition as defined claim 13.
